SEQUENCE LISTING (35 sequences)

SEQ ID NO: 1 - DNA, 5134 bp, Rattus norvegicus
ggaaaagacagagccagcctcggaggagcaggagccggcagaagacacagaccaggccaggttgtcagcagactacgagaaggtggagctgcctttggaagaccaggttggtgacctggaggcatcgtcagaggagaagtgtgctcctttggcaacggaagtgtttgatgagaagatggaagcccaccaagaagttgttgcagaggtccacgtgagcaccgtggagaagacagaggaggagcagggaggaggaggagaggctgaagggggcgtggtggtagaaggaacaggagaatccttgccccctgagaaactggctgagccccaggaggtcccccaggaagctgagcctgctgaggagctgatgaagagcagagagatgtgtgtctctggaggagaccacactcaactgacagacctaagtcctgaagagaagacgctgcccaaacacccagaaggcattgtcagtgaggtggagatgctgtcctctcaggaaagaatcaaggtacagggaagtcccttgaagaaactcttcagtagctcaggcttaaagaagctgtctgggaagaagcagaaggggaaacgaggaggtgggggagacgaagagcctggagaataccaacacattcacaccgaatccccagagagtgctgatgagcagaagggagagagctctgcgtcgtcccccgaggagcctgaggagaccacgtgtctggagaaagggccgctggaagcacccaggatggggaagctgaggaaggaactacttcgtggagagaagaagaggaaggatcactccctgggcatccttcaaaaagatggtgacacccaagaaacggtccgaagaccttctgagagtgacaaggaggaagagctggagaaggtcaagagcgccaccttgtcctccactgatagcacagtgtcagaaatgcaagatgaagtcaaaactgttggtgaggaacaaaagccagaggaaccaaagcgtagggtggatacttcagtgtcttgggaagcactgatttgtgtcggatcatccaagaagagagcaaggaaggcatcctcttcagatataagagggccaaggacactgggagggggacagtcacagagcagaggaggccagcaaagacaaagaagccgaacagacgctgttcctgccagcacccaggagcaggaccaagcgcaaggaagttcctcacccgagccagcgggaagcccttccgaaggggaaggtgtctccacttgggagtcatttaaaagattagtcactccaagaaaaaaatccaagtcaaaactggaagagaaagaagccggaaggactctagttgtaggagcaggttgtccactgagatcgaaccgtgtagagaagaatcttgggtttccattaagaaattcatccccggacggcggaagaaaagggcagatgggaaggcaagaacaagccactgtggaagactcagggccagtggagataaatgaggacgagcctgatgtcccagccgtcgtgcctctgtctgagtatgatgcagtggagagggagaagatggaagcccaggggaatgcggagctgcccagctgctggggctgtgtagtgtccgaggagctcagtaagactctggtccacactgtgagtgtcgcagtcattgatgggaccagggcagtcaccagtgtcgaagagcggtctccttcgtggatatccgcttccgtaacagaacctcttgaacacacagcgggagaagccatgccacctgttgaagaggtcactgaaaaagacatcattgcagaagaaactcctgtgctcacccagacgttaccagagggtaaagatgcccatgacgacatggtcaccagtgaagtggatttcacctcagaagctgtgacagccacagagacctcagaggctctccgtactgaagaagttaccgaagcatcgggggccgaagagaccacagacatggtgtccgcagtttcccagctgactgactccccagacaccacagaggaagccaccccagttcaggaggtagagggtggtgtgctagatacagaagaagaggagcgccagacgcaggccatcctccaagccgttgcagacaaggtgaaagaggagtcccaggtgcctgcaacccagactgtgcagagaacggggtcaaaagcactggagaaggttgaggaggtagaggaggactccgaagtgctggcttcggagaaagagaaggacgttatgccgaaaggacccgtgcaggaagctggagctgagcatcttgcacagggctctgagactggacaggctactccagagagccttgaagttcctgaagtcacagcagatgtagaccatgtcgccacgtgccaggttatcaagctccagcagctgatggaacaggccgtggcccctgagtcatccgaaaccttgacagacagtgagacaaatggaagcactcccttagcagattcagacactgcagatgggacacagcaagatgaaaccattgacagccaggacagtaaagccactgcagctgtcaggcagtcacaggtcacagaagaagaggcggctactgctcagaaagaggagccttcgacactacctaataatgttccagcccaggaagaacatggggaagaaccaggaagagatgttcttgaacctacacagcaagagcttgctgctgcagccgtgcccgtctggcaaaagactgaggtgggtcaagagggtgaggttgactggttggatggagaaaaagtcaaagaagaacaggaggtgtttgtacactctggacccaacagtcaaaaggctgctgatgtgacatatgacagtgaagtgatgggagtggccgggtgtcaggaaaaggagagtactgaagtgcagagtcttagcctggaggagggagagatggaaactgacgttgaaaaggagaaaagggagacaaagccagagcaagtgagtgaagaaggtgagcaggaaacagccgctcctgagcatgaaaggaactacgggaagccagtcctgacacttgacatgcccagctcagagagggggaaggcactgggaagccttggaggaagcccttctctcccagaccaagacaaagcaggttgcatagaggttcaagttcaaagcctggacacaacagtcactcaaacagcagaagctgtggaaaaggtcatagaaacggttgtgatttcagagacaggtgaaagtccagagtgtgtaggtgcacacttattaccagctgagaagtcctctgcaacgggtggccactggactcttcagcatgcagaggacacggtacccctggggcctgagtctcaggcagaatccatcccaatcatagtaactcctgctcctgaaagcaccctacatcctgacctacaaggagaaataagcgcatcccagagagagcgatcagaggaagaggacaagccagatgctggtcctgatgctgacggcaaggagagtacagcaatcgacaaagtcctcaaggctgaacctgagatcctggaacttgagagtaagagcaacaagattgtgctgaacgtcattcagacagccgttgaccagttcgcacgtacagaaacagcccccgaaactcatgcttatgattcacagacccaggttcctgcaatgcgcttggacagcagggagcccaacagatgctggacaaaaatgaaagttgccaagatgaaacacccagtgccgcagcccagagaggacttgcaagtcctgaccgttctggaggcatggctcagctcggaaatgcttgccgcgcttgcagttgaaagcgccggtgtcaaagtaagcattgagaagctgcctcctcaacccaaagatcaaaaggagcatgctgctgatggccctcagctccaaagcttagcccaggcagaggcagtgtctggaaacctaaccaaagaatccccagacaccaacggaccaaagctaaccgaggagcgatgcccccaaaagttgaggtccaggaagaagaaatgtctaccaagtcagtcaaagagaacaaggcccaggcagaagaggacctgcaggagccaaagggagacctggcagaatcctaagatgttagttgctcattgtacatctgtaagaccagaatgtgaaaacaagtcacagaacaagatgctgctgttgggaccttggaccaagatttcagagcccatgagatccagagagcagggccgtccaatgatttccacccagtagagcaccccgacaattctgaggcttcatcgggagctagagccagctaacatttcctcgtttcaagactgcctttgatttgccccttgatgccgtccgtgtatttctaacatttcctcgtttcaagactgcctttgatttgccccttgatgccgtccgtgtatttcggatttaaggtcctgcgttctcaacctggaaccaattctgccatacctagttccacttctcaaactggagcatcctcctttatgtatttatatgtatgttttatgtagtcctcctcctgtacctattgtatatttttttctaacgtttaagcacatgctttttgtattatgcaatatataacgggtgtgcagccatagcgacgctttgaaaagctccaagcctcaactgtaacctgcagcaaacagataacattcctggcaagaagagacaagtcttttttaaagtttactgatgcttagatctgtgggcttctagtcctctgaaagtggttgttttcctatgcacagcgagctcagaaataaaaaccccattttgaaacatccaggatgtcccaatattaccatgattttttcccccctttttgctaatccagtccaggttggaaagaagtctcctctgtgtcagattaagccctgtctcttaatgatatggacaaatgagtgtgcctaaggccatgagatgtttcctaatgcagaaggaatctgttgtacgtttttttgattgtactcttctatgctggaccgaattcatatgcagatcgaagtgagtcctgttctttacagatggtattttgatagatactggagtttgtctgtgttatatctgtgccccttctttaagaacaatgttgcattatgttcctttggataaattgtgatttgacaactgatttaaataaacatatttgactac

SEQ ID NO: 2 - protein, 1346 aa, Rattus norvegicus
MEAHQEVVAEVHVSTVEKTEEEQGGGGEAEGGVVVEGTGESLPPEKLAEPQEVPQEAEPAEELMKSREMCVSGGDHTQLTDLSPEEKTLPKHPEGIVSEVEMLSSQERIKVQGSPLKKLFSSSGLKKLSGKKQKGKRGGGGDEEPGEYQHIHTESPESADEQKGESSASSPEEPEETTCLEKGPLEAPRMGKLRKELLRGEKKRKDHSLGILQKDGDTQETVRRPSESDKEEELEKVKSATLSSTDSTVSEMQDEVKTVGEEQKPEEPKRRVDTSVSWEALICVGSSKKRARKASSSDIRGPRTLGGGQSQSRGGQQRQRSRTDAVPASTQEQDQAQGSSSPEPAGSPSEGEGVSTWESFKRLVTPRKKSKSKLEEKEAGRTLVVGAGCPLRSNRVEKNLGFPLRNSSPDGGRKGQMGRQEQATVEDSGPVEINEDEPDVPAVVPLSEYDAVEREKMEAQGNAELPSCWGCVVSEELSKTLVHTVSVAVIDGTRAVTSVEERSPSWISASVTEPLEHTAGEAMPPVEEVTEKDIIAEETPVLTQTLPEGKDAHDDMVTSEVDFTSEAVTATETSEALRTEEVTEASGAEETTDMVSAVSQLTDSPDTTEEATPVQEVEGGVLDTEEEERQTQAILQAVADKVKEESQVPATQTVQRTGSKALEKVEEVEEDSEVLASEKEKDVMPKGPVQEAGAEHLAQGSETGQATPESLEVPEVTADVDHVATCQVIKLQQLMEQAVAPESSETLTDSETNGSTPLADSDTADGTQQDETIDSQDSKATAAVRQSQVTEEEAATAQKEEPSTLPNNVPAQEEHGEEPGRDVLEPTQQELAAAAVPVWQKTEVGQEGEVDWLDGEKVKEEQEVFVHSGPNSQKAADVTYDSEVMGVAGCQEKESTEVQSLSLEEGEMETDVEKEKRETKPEQVSEEGEQETAAPEHERNYGKPVLTLDMPSSERGKALGSLGGSPSLPDQDKAGCIEVQVQSLDTTVTQTAEAVEKVIETVVISETGESPECVGAHLLPAEKSSATGGHWTLQHAEDTVPLGPESQAESIPIIVTPAPESTLHPDLQGEISASQRERSEEEDKPDAGPDADGKESTAIDKVLKAEPEILELESKSNKIVLNVIQTAVDQFARTETAPETHAYDSQTQVPAMRLDSREPNRCWTKMKVAKMKHPVPQPREDLQVLTVLEAWLSSEMLAALAVESAGVKVSIEKLPPQPKDQKEHAADGPQLQSLAQAEAVSGNLTKESPDTNGPKLTEERCPQKLRSRKKKCLPSQSKRTRPRQKRTCRSQRETWQNPKMLVAHCTSVRPECENKSQNKMLLLGPWTKISEPMRSREQGRPMISTQ

SEQ ID NO: 3 - DNA, 6160 bp, Rattus norvegicus
cgggccggcgggagtagaagagccactgagccatgggcgcaggcagttccaccgagcagcggagccccgagcagccggcggggagcgacacgccgagcgagctggtgctcagtggccatgggcccgcagctgaagcctcgggagcagctggagaccccgccgacgcggaccccgccaccaagctcccacagaagaatggccagctgtcttctgtcaacggcgtagctgaacaaggagatgtccatgtccaagaggaaaaccaggaggggcaggaggaagaagtcgttgatgaggatgttggacagcgagagtcagaagatgtgagagaaaaagaccgagttgaagaaatggcggccaactccacagctgttgaagatatcacaaaggatgggcaggaggagacatcagaaataattgaacagatccctgcttcagaaaacaatgtggaagaaatggtacagcctgctgagtcccaggctaatgatgttggcttcaagaaagtatttaaatttgttggttttaaattcacggtgaagaaggataaaaatgaaaagtcagatactgtccaactactcactgtcaagaaggatgaaggcgaaggggcagaagcctctgtcggagctggagaccaccaggagcccagtgtggagactgccgtcggagagtcagcatccaaagaaagtgagctgaagcaatccacagagaagcaagaaggcaccctgaagcaagaacagagcagcacagaaatcccccttcaagccgaatctgatcaagcggctgaggaagaagccaaagatgaaggagaagaaaaacaagagaaagagcccaccaagtccccagaatccccgagcagcccagtcaacagtgagacaacatcttccttcaagaagttcttcactcacggttgggccggctggcgcaagaagaccagcttcaagaaatcaaaagaggatgatctggaaactgccgagaagagaaaggagcaagaggcagaaaaagtagacgaggaagaaaaggaaaagacagagccagcctcggaggagcaggagccggcagaagacacagaccaggccaggttgtcagcagactacgagaaggtggagctgcctttggaagaccaggttggtgacctggaggcatcgtcagaggagaagtgtgctcctttggcaacggaagtgtttgatgagaagatggaagcccaccaagaagttgttgcagaggtccacgtgagcaccgtggagaagacagaggaggagcagggaggaggaggagaggctgaagggggcgtggtggtagaaggaacaggagaatccttgccccctgagaaactggctgagccccaggaggtcccccaggaagctgagcctgctgaggagctgatgaagagcagagagatgtgtgtctctggaggagaccacactcaactgacagacctaagtcctgaagagaagacgctgcccaaacacccagaaggcattgtcagtgaggtggagatgctgtcctctcaggaaagaatcaaggtacagggaagtcccttgaagaaactcttcagtagctcaggcttaaagaagctgtctgggaagaagcagaaggggaaacgaggaggtgggggagacgaagagcctggagaataccaacacattcacaccgaatccccagagagtgctgatgagcagaagggagagagctctgcgtcgtcccccgaggagcctgaggagaccacgtgtctggagaaagggccgctggaagcaccccaggatggggaagctgaggaaggaactacttccgatggagagaagaagagagaagggatcactccctgggcatccttcaaaaagatggtgacacccaagaaacgggtccgaagaccttctgagagtgacaaggaggaagagctggagaaggtcaagagcgccaccttgtcctccactgatagcacagtgtcagaaatgcaagatgaagtcaaaactgttggtgaggaacaaaagccagaggaaccaaagcgtagggtggatacttcagtgtcttgggaagcactgatttgtgtcggatcatccaagaagagagcaaggaaggcatcctcttcagatgatgaaggagggccaaggacactgggaggggacagtcacagagcagaggaggccagcaaagacaaagaagccggaacagacgctgttcctgccagcacccaggagcaggaccaagcgcaaggaagttcctcacccgagccagcgggaagcccttccgaaggggaaggtgtctccacttgggagtcatttaaaagattagtcactccaagaaaaaaatccaagtcaaaactggaagagaaagccgaagactctagtgtagagcagttgtccactgagatcgaaccgagtagagaagaatcttgggtttccattaagaaattcatccccggacggcggaagaaaagggcagacgggaagcaagaacaagccactgtggaagactcagggccagtggagataaatgaggacgaccctaatgtcccagccgtcgtgcctctgtctgagtataatgcagtggagagggagaagatggaagcccaggggaatacggagctgccccagctgctgggggctgtgtacgtgtccgaggagctcagtaagactctggtccacactgtgagtgtcgcagtcattgatgggaccagggcagtcaccagtgtcgaagagcggtctccttcgtggatatccgcttccgtaacagaacctcttgaacacacagcgggagaagccatgccacctgttgaagaggtcactgaaaaagacatcattgcagaagaaactcctgtgctcacccagacgttaccagagggtaaagatgcccatgacgacatggtcaccagtgaagtggatttcacctcagaagctgtgacagccacagagacctcagaggctctccgtactgaagaagttaccgaagcatcgggggccgaagagaccacagacatggtgtccgcagtttcccagctgactgactccccagacaccacagaggaagccaccccagttcaggaggtagagagtggtgtgctagatacagaagaagaggagcgccagacgcaggccatcctccaagccgttgcagacaaggtgaaagaggagtcccaggtgcctgcaacccagactgtgcagagaacggggtcaaaagcactggagaaggttgaggaggtagaggaggactccgaagtgctggcttcggagaaagagaaggacgttatgccgaaaggacccgtgcaggaagctggagctgagcatcttgcacagggctctgagactggacaggctactccagagagccttgaagttcctgaagtcacggcagatgtagaccatgtcgccacgtgccaggttatcaagctccagcagctgatggaacaggccgtggcccctgagtcatccgaaaccttgacagacagtgagacaaatggaagcactcccttagcagattcagacactgcagatgggacacagcaagatgaaaccattgacagccaggacagtaaagccactgcagctgtcaggcagtcacaggtcacagaagaagaggcggctactgctcagaaagaggagccttcgacactacctaataatgttccagcccaggaagaacatggggaagaaccaggaagagatgttcttgaacctacacagcaagagcttactgctgcagccgtgcccgttctggcaaagactgaggtgggtcaagagggtgaggttgactggttggatggagaaaaagtcaaagaagaacaggaggtgtttgtacactctggacccaacagtcaaaaggctgctgatgtgacatatgacagtgaagtgatgggagtggccgggtgtcaggaaaaggagagtactgaagtgcagagtcttagcctggaggagggagagatggaaactgacgttgaaaaggagaaaagggagacaaagccagagcaagtgagtgaagaaggtgagcaggaaacagccgctcctgagcatgaaggaacctacgggaagccagtcctgacacttgacatgcccagctcagagagggggaaggcactgggaagccttggaggaagcccttctctcccagaccaagacaaagcaggttgcatagaggttcaagttcaaagcctggacacaacagtcactcaaacagcagaagctgtggaaaaggtcatagaaacggttgtgatttcagagacaggtgaaagtccagagtgtgtaggtgcacacttattaccagctgagaagtcctctgcaacgggtggccactggactcttcagcatgcagaggacacggtacccctggggcctgagtctcaggcagaatccatcccaatcatagtaactcctgctcctgaaagcaccctacatcctgacctacaaggagaaataagcgcatcccagagagagcgatcagaggaagaggacaagccagatgctggtcctgatgctgacggcaaggagagtacagcaatcgaaaaagtcctcaaggctgaacctgagatcctggaacttgagagtaagagcaacaagattgtgctgaacgtcattcagacagccgttgaccagttcgcacgtacagaaacagcccccgaaactcatgcttatgattcacagacccaggttcctgcatgcaggcttgacagcagggagcccaacagatgctggacaaaaatgaaagatgccaagatgaaacacccagtgccgcagcccagagaggacttgcaagtcctgaccgttctggaggcatgggctcagcctcggaaatgcttgccgcgcttgcagttgaaagcgccggtgtcaaagtaagcattgagaagctgcctcctcaacccaaagatccaaaaggagcatgctgctgatggccctcagctccaaagcttagcccaggcagaggccagtgcctctggaaacctaaccaaagaatccccagacaccaacggaccaaagctaaccgaggagggcgatcccccaaaagttgaggtccaggaagaagaaatgtctaccaagtcagtcaaagagaacaaggcccaggcagaagaggacctgcaggagccaaagggagacctggcagaatcctaagatgttagttgctcattgtacatctgtaagaccagaatgtgaaaacaagtcacagaacaagatgctgctgttgggaccttgagaccaagatttcagagcccatgagatccagagagcagggccgtccaatgatttccacccagtagagcaccccgacaattctgaggcttcatcgggagctagagccagctaacatttcctcgtttcaagactgcctttgatttgccccttgatgccgtccgtgtatttctaacatttcctcgtttcaagactgcctttgatttgccccttgatgccgtccgtgtatttcggatttaaggtcctgcgttctcaacctggaaccaattctgccatacctagttccacttctcaaactggagcatcctcctttatgtatttatatgtatgttttatgtagtcctcctcctgtacctattgtatatttttttctaacgtttaagcacatgctttttgtattatgcaatatataacgggtgtgcagccatagcgacgctttgaaaagctccaagcctcaactgtaacctgcagcaaacagataacattcctggcaagaagagacaagtcttttttaaagtttactgatgcttagatctgtgggcttctagtcctctgaaagtggttgttttcctatgcacagcgagctcagaaataaaaaccccattttgaaacatccaggatgtcccaatattaccatgattttttcccccctttttgctaatccagtccaggttggaaagaagtctcctctgtgtcagattaagccctgtctcttaatgatatggacaaatgagtgtgcctaaggccatgagatgtttcctaatgcagaaggaatctgttgtacgtttttttgattgtactcttctatgctggaccgaattcatatgcagatcgaagtgagtcctgttctttacagatggtattttgatagatactggagtttgtctgtgttatatctgtgccccttctttaagaacaatgttgcattatgttcctttggataaattgtgatttgacaactgatttaaataaacatatttgactac

SEQ ID NO: 4 - protein, 1596 aa, Rattus norvegicus
MGAGSSTEQRSPEQPAGSDTPSELVLSGHGPAAEASGAAGDPADADPATKLPQKNGQLSSVNGVAEQGDVHVQEENQEGQEEEVVDEDVGQRESEDVREKDRVEEMAANSTAVEDITKDGQEETSEIIEQIPASENNVEEMVQPAESQANDVGFKKVFKFVGFKFTVKKDKNEKSDTVQLLTVKKDEGEGAEASVGAGDHQEPSVETAVGESASKESELKQSTEKQEGTLKQEQSSTEIPLQAESDQAAEEEAKDEGEEKQEKEPTKSPESPSSPVNSETTSSFKKFFTHGWAGWRKKTSFKKSKEDDLETAEKRKEQEAEKVDEEEKEKTEPASEEQEPAEDTDQARLSADYEKVELPLEDQVGDLEASSEEKCAPLATEVFDEKMEAHQEVVAEVHVSTVEKTEEEQGGGGEAEGGVVVEGTGESLPPEKLAEPQEVPQEAEPAEELMKSREMCVSGGDHTQLTDLSPEEKTLPKHPEGIVSEVEMLSSQERIKVQGSPLKKLFSSSGLKKLSGKKQKGKRGGGGDEEPGEYQHIHTESPESADEQKGESSASSPEEPEETTCLEKGPLEAPQDGEAEEGTTSDGEKKREGITPWASFKKMVTPKKRVRRPSESDKEEELEKVKSATLSSTDSTVSEMQDEVKTVGEEQKPEEPKRRVDTSVSWEALICVGSSKKRARKASSSDDEGGPRTLGGDSHRAEEASKDKEAGTDAVPASTQEQDQAQGSSSPEPAGSPSEGEGVSTWESFKRLVTPRKKSKSKLEEKAEDSSVEQLSTEIEPSREESWVSIKKFIPGRRKKRADGKQEQATVEDSGPVEINEDDPNVPAVVPLSEYNAVEREKMEAQGNTELPQLLGAVYVSEELSKTLVHTVSVAVIDGTRAVTSVEERSPSWISASVTEPLEHTAGEAMPPVEEVTEKDIIAEETPVLTQTLPEGKDAHDDMVTSEVDFTSEAVTATETSEALRTEEVTEASGAEETTDMVSAVSQLTDSPDTTEEATPVQEVESGVLDTEEEERQTQAILQAVADKVKEESQVPATQTVQRTGSKALEKVEEVEEDSEVLASEKEKDVMPKGPVQEAGAEHLAQGSETGQATPESLEVPEVTADVDHVATCQVIKLQQLMEQAVAPESSETLTDSETNGSTPLADSDTADGTQQDETIDSQDSKATAAVRQSQVTEEEAATAQKEEPSTLPNNVPAQEEHGEEPGRDVLEPTQQELTAAAVPVLAKTEVGQEGEVDWLDGEKVKEEQEVFVHSGPNSQKAADVTYDSEVMGVAGCQEKESTEVQSLSLEEGEMETDVEKEKRETKPEQVSEEGEQETAAPEHEGTYGKPVLTLDMPSSERGKALGSLGGSPSLPDQDKAGCIEVQVQSLDTTVTQTAEAVEKVIETVVISETGESPECVGAHLLPAEKSSATGGHWTLQHAEDTVPLGPESQAESIPIIVTPAPESTLHPDLQGEISASQRERSEEEDKPDAGPDADGKESTAIEKVLKAEPEILELESKSNKIVLNVIQTAVDQFARTETAPETHAYDSQTQVPACRLDSREPNRCWTKMKDAKMKHPVPQPREDLQVLTVLEAWAQPRKCLPRLQLKAPVSK

SEQ ID NO: 5 - protein, 10 aa, Artificial Sequence
  Myristylation site for src protein
MGSSKSKPKD

SEQ ID NO: 6 - protein, 10 aa, Artificial Sequence
  Myristylation and palmitylation site for yes protein
MGCIKSKEDK

SEQ ID NO: 7 - protein, 10 aa, Artificial Sequence
  Myristylation and/or palmitylation site for SSeCKS protein
MGAGSSTEQR

SEQ ID NO: 8 - protein, 10 aa, Artificial Sequence
  Myristylation site for G protein
MGAGASAEEK

SEQ ID NO: 9 - protein, 10 aa, Artificial Sequence
  Myristylation and palmitylation site for G protein
MGCTLSAEDK

SEQ ID NO: 10 - protein, 10 aa, Artificial Sequence
  Palmitylation site for GAP-43 protein
MLCCMRRTKQ

SEQ ID NO: 11 - protein, 23 aa, Bos taurus
KRFSSKKSFKLSGFSFKKNKKEA

SEQ ID NO: 12 - protein, 23 aa, Mus musculus
KRFSSKKSFKLSGFSFKKSKKEA

SEQ ID NO: 13 - protein, 17 aa, Unknown
  Phosphorylation consensus site for MacMARCKS/F52 protein
KKFSSKKPFKLSGFSFR

SEQ ID NO: 14 - protein, 20 aa, Unknown
  Calmodulin binding domain for myosin light chain kinase protein
KRRWKKAFIAVSAAARFKKC

SEQ ID NO: 15 - protein, 23 aa, Rattus norvegicus
ETTSSFKKFFTHGTSFKKSKEDD

SEQ ID NO: 16 - protein, 23 aa, Rattus norvegicus
KLFSSSGLKKLSGKKQKGKRGGG

SEQ ID NO: 17 - protein, 23 aa, Rattus norvegicus
EGITPWASFKKMVTPKKRVRRPS

SEQ ID NO: 18 - protein, 23 aa, Rattus norvegicus
EGVSTWESFKRLVTPRKKSKSKL

SEQ ID NO: 19 - protein, 20 aa, Artificial Sequence
  SSeCKS phosphorylation consensus sequence
EGXXXWXSFKXXVTPXKXXX

SEQ ID NO: 20 - protein, 7 aa, Artificial Sequence
  consensus sequence
WAGWRKK

SEQ ID NO: 21 - protein, 54 aa, Notophthalmus viridescens
  VARIANT (20)...(46): any amino acid
SPLKSPYKHPEGLLSPTKMXXXXXXXXXXXXXXXXXXXXXXXXXXXLSSSERLR

SEQ ID NO: 22 - protein, 14 aa, Rattus norvegicus
  VARIANT (6)...(9): any amino acid
KKLFSXXXXKKLSG

SEQ ID NO: 23 - protein, 10 aa, Rattus norvegicus
MGAGSSTEQR

SEQ ID NO: 24 - DNA, 26 bp, Artificial Sequence
  Oligonucleotide primer
gtgactggtgaggcctcaaccaagtc

SEQ ID NO: 25 - DNA, 26 bp, Artificial Sequence
  Oligonucleotide primer
gtgactggtgagtactcaaccaagtc

SEQ ID NO: 26 - DNA, 30 bp, Artificial Sequence
  Oligonucleotide primer
ggaagtcccttgtcgagcctcttcagtagc

SEQ ID NO: 27 - DNA, 26 bp, Artificial Sequence
  Oligonucleotide primer
gctcaggcttaagctcgctgtctggg

SEQ ID NO: 28 - DNA, 24 bp, Artificial Sequence
  Oligonucleotide primer
cccttgaagaaaagcttcagtagc

SEQ ID NO: 29 - DNA, 24 bp, Artificial Sequence
  Oligonucleotide primer
ggcttaaagaagtcgtctgggaag

SEQ ID NO: 30 - DNA, 24 bp, Artificial Sequence
  Oligonucleotide primer
cccttgtcgagcagcttcagtagc

SEQ ID NO: 31 - DNA, 24 bp, Artificial Sequence
  Oligonucleotide primer
ggcttaagctcgtcgtctgggaag

SEQ ID NO: 32 - protein, 16 aa, Artificial Sequence
  Penetratin peptide
RQIKIWFQNRRMKWKK

SEQ ID NO: 33 - protein, 16 aa, Artificial Sequence
  SSeCKS cyclin binding site
LKKLFSSSGLKKLSGK

SEQ ID NO: 34 - protein, 16 aa, Artificial Sequence
  SSeCKS mutated cyclin binding site
LSSSFSSSGLSSSSGK

SEQ ID NO: 35 - protein, 16 aa, Artificial Sequence
  SSeCKS mutated phosphoserine cyclin binding site
LKKLFSSSGLKKLSGK

(19) United States
(12) Patent Application Publication
(10) Pub. No.: US 2005/0260693 A1
(43) Pub. Date: Nov. 24, 2005

(54) Tumor suppressor gene

(76) Inventor: Irwin Gelman, New York, NY (US)

Correspondence Address: BAKER & BOTTS, 30 ROCKEFELLER PLAZA, NEW YORK, NY, 10112, US

(21) Appl. No.: 11/060,005

(22) Filed: Jul. 19, 2005

Related U.S. Application Data
(62) Division of application No. 09/902,432, filed on Jul. 10, 2001, now abandoned.
Application No. 09/902,432 is a continuation-in-part of application No. 08/978,277, filed on Nov. 25, 1997, now Pat. No. 6,582,956.
Application No. 08/978,277 is a continuation of application No. 08/665,401, filed on Jun. 18, 1996, now abandoned.
Application No. 08/665,401 is a continuation of application No. 08/635,121, filed on Apr. 19, 1996, now Pat. No. 5,910,442.

(51) Int. Cl. C12Q 1/68; G01N 33/574; C12N 5/06; C07K 16/30

(52) U.S. Cl. 435/7.23; 530/388.8; 435/338

(57) ABSTRACT

The present invention relates to a novel tumor suppressor gene, referred to herein as SSeCKS, its encoded protein, and methods of use thereof. It is based, at least in part, on the discovery of a SSeCKS gene which encodes a substrate of protein kinase C that functions as both a mitogenic regulator as well as a tumor suppressor.

### 1. INTRODUCTION

The present invention relates to a novel tumor suppressor gene, referred to herein as SSeCKS, its encoded protein, and methods of use thereof. It is based, at least in part, on the discovery of a SSeCKS gene which encodes a substrate of protein kinase C that functions as both a mitogenic regulator as well as a tumor suppressor.

### 2. BACKGROUND OF THE INVENTION

The inactivation of several tumor suppressor gene families (for example, those encoding p53, Rb, and APC) as a result of mutation is acknowledged to contribute to oncogenicity of several types of human cancers (Levine, 1993, Ann. Rev. Biochem. 62:623-651). Many of these so-called class I tumor suppressor genes (Lee et al., 1991, Proc. Natl. Acad. Sci. U.S.A. 88:2825-2829) were identified and isolated following cumbersome pedigree and cytogenetic analyses (Sager, 1989, Science 246:1406-1412). Recently, another class of genes (class II) whose expression is known to be down regulated in tumor cells has been shown by gene transfer techniques to encode potential tumor suppressors. These include non-muscle α-actinin, tropomyosin I, CLP, retinoic acid receptor β1, and interferon regulatory factor (Gluck et al., 1993, Proc. Natl. Acad Sci. U.S.A. 90:383-387; Hirada et al., 1993, Science 259:971-974; Hogel et al., 1993, Proc. Natl. Acad. Sci. U.S.A. 90:985-989; Mishra et al., 1994, J. Cell. Biochem. 18(Supp. C):171; Plasad et al., 1993, Proc. Natl. Acad. Sci. U.S.A. 90:7039-7043). Addi-tional tumor suppressor gene families such as the maspin gene, rrg, and NO3 (Contente et al., 1993, Science 249:796-798; Ozaki et al., 1994, Cancer Res. 54:646-648; Zou et al., 1994, Science 263:526-529) were isolated by subtractive hybridization techniques designed to identify down-regulated genes. The ability of these genes to reverse an array of oncogenic phenotypes following gene transfer and over expression supports the possibility for novel therapeutic modalities for cancer.

### 3. SUMMARY OF THE INVENTION

The present invention relates to a novel tumor suppressor gene, SSeCKS. It is based, at least in part, on the discovery of a gene, hitherto referred to as “322” (Lin et al., 1995, Mol. Cell. Biol. 15:2754-2762) but now referred to as SSeCKS, which was found to be down-regulated in certain transformed cells. Further, the SSeCKS gene product has been found to be a substrate of protein kinase C, and has been shown to act as a mitogenic regulator and as an inhibitor of the transformed phenotype.

In addition, the present invention relates to the discovery that SSeCK controls progression of cells through the G1 through S phase of the cell cycle by regulating the expression and localization of cyclin D activity. Thus, the invention further relates to methods for identifying compounds capable of modulating SSeCKS mediated cyclin D activity.

In various embodiments, the present invention relates to the SSeCKS gene and protein, and in particular, to rat and human SSeCKS gene and protein. Furthermore, the present invention provides for the use of such genes and proteins in diagnostic and therapeutic methods. In particular, the invention relates to assays designed for measuring the metastatic potential of isolated cancer cells based on detection of SSeCK expression. The invention is based on the observation that loss of SSeCKS expression was found to correlate with the metastatic progression of human prostate cancer.

### 4. BRIEF DESCRIPTION OF THE DRAWINGS

FIG. 1. Northern blot analysis of SSeCKS RNA levels in NIH 3T3 cells versus NIH/v-src transformed cells.

FIG. 2. Southern blot analysis showing that the decreased level of SSeCKS RNA in NH/v-src cells is not due to gross deletion or translocation of the SSeCKS allele, and restriction map of SSeCKS.

FIG. 3. Nucleic acid (top line, lower case letters; SEQ ID NO:1) and deduced amino acid (lower line, capital letters; SEQ ID NO:2) sequence of rat SSeCKS cDNA encoding an active truncated form of SSeCKS.

FIG. 4. Northern blot analysis showing that the transcription of SSeCKS is suppressed relatively soon after the activation of a ts-src allele (A) or the addition of fetal calf serum (FCS) to starved rodent fibroblasts (B).

FIG. 5. Northern blot analyses showing levels of SSeCKS transcripts in oncogene-transformed Rat-6 fibroblasts.

FIG. 6. Results of in vitro transcription-translation of SSeCKS cDNA.

FIG. 7. Proliferation of cells overexpressing SSeCKS.

FIG. 8. “Zoo” Southern blot of SSeCKS probe to genomic DNA from various species.

FIG. 9. Northern blot analysis showing tissue-specific expression of SSeCKS in mice.

FIG. 10. Schematic diagram of SSeCKS protein.

FIG. 11A-I. Nucleic acid sequence of rat cDNA (SEQ ID NO:3) encoding full-length SSeCKS and deduced amino acid sequence (SEQ ID NO:4).

FIG. 12. In vitro transcription and translation of SSeCKS. One μg of plasmid DNA encoding the full-length SSeCKS cDNA or a N-terminally truncated SSeCKS cDNA (clone 13.2.2) were incubated in a coupled T7 transcription/trans-lation reaction (TNT; Promega) containing [³⁵S]-methionine as described in section 7.1. One tenth of the labeled products were analyzed by SDS-PAGE followed by fluorography. Protein size markers are shown at left. Note that a shortened version of SSeCKS, synthesized from an internal ATG start site in clone 13.2.2, is not produced in the context of the upstream ATG start site in the full-length SSeCKS cDNA in in vitro reactions.

FIG. 13A-C. Glutathione S-transferase fusion constructs of SSeCKS domains. Secondary structural analysis of SSeCKS predicted a rod-like molecule with a high degree of hydrophilicity and amphipathic helices, and a concentration of Chou-Fasman turns (Chou and Fasman, 1978, Advances in Enzymology 47:45-147) from residues 400-900 (13B and 13C). The turns in this region were not recognized by the Robson-Garnier algorithm (Gamier et al., 1978, J. Mol. Biol. 120:97-120), as shown in 13C. Four concentrations of predicted PKC phosphorylation sites (S/TXK/R or K/RXXS/T) were also identified (13A, white boxes; numbered 1-4). The black bars (13A) indicate the sizes and names of GST-SSeCKS fusion constructs containing individual or combinations of the predicted PKC sites.

FIG. 14A-B. In vivo phosphorylation of SSeCKS by PKC. Confluent Rat-6 cells grown overnight in DEM lacking calf serum were starved of phosphate for 2 hours and then labeled for 4 hours with [³²P]orthophosphate. At the end of the labeling period, some cells were treated with 200 nM PMA (lane b, 2 min; lanes c and d, 15 min) and the PKC-specific inhibitor, bis-indolylmaleimide (lane d, 30 min). SSeCKS protein was immunoprecipitated from equal aliquots (400 μg) of lysates from untreated (lane a) or treated cells (lanes b-d), and western blotted onto a PVDF membrane (14A). 14B represents immunoblotting using rabbit anti-SSeCKS serum (showing equal amounts of SSeCKS protein loaded) whereas the upper panel represents autoradiography of the blotted protein (showing an increase in ³²PO₄-labeling of SSeCKS following PMA treatment). The 280/290 kDa doublet (unresolved in this gel) is indicated by an arrow, and the minor 240 kDa form of SSeCKS can be detected in the upper panel. A better resolution of these SSeCKS species is shown in FIG. 22.

FIG. 15A-B. In vitro phosphorylation of SSeCKS by PKC. GST and GST/322 fusion protein (see FIG. 13) were expressed and purified from bacteria as described in section 7.1 (15A). Five μg of the GST samples were added to PKC assays containing [³²P]-δ-ATP in the presence or absence of the PKC peptide inhibitor. The products were then bound to glutathione-Sepharose beads, precipitated and washed, and analyzed by SDS-PAGE and auto-radiography (15B). Protein size markers are indicated on the appropriate sides. Radioactive-labeling was detected in GST-322 (160 kDa) only.

FIG. 16A-B. Phospholipid preference for the in vitro phosphorylation of SSeCKS by PKC. Myelin basic protein, MBP (16A), GST-322 and GST proteins (16B) were phosphorylated in vitro as in FIG. 15, in the presence or absence of various lipids including phosphatidylserine (PS), phosphatidylcholine (PC) or phosphatidylinositol (PI). In some cases, excess PKC peptide inhibitor was added as in FIG. 15. The extent of labeling in the peptide substrates was determined by spotting the reaction products on phosphocellulose discs (Whatman), precipitating peptides with washes of 5% trichloroacetic acid, followed by scintillation counting.

FIG. 17A-B. Co-precipitation of SSeCKS with PKC. 17A: GST-1322 fusion protein (see FIG. 13) was expressed and purified from bacteria as described in section 7.1. 17B: RIPA lysates (1 mg protein per sample) from Rat-6 or Rat-6/PKC-α overexpressor cells, or purified rabbit brain PKC (20 ng; “P-PKC-α”) were incubated for 4 h at 4° C. with fifty μg of GST-1322 (or GST alone) in the presence or absence of 0.37 mg/ml phosphatidylserine (PS). The proteins were then precipitated with glutathione-Sepharose beads, washed and western blotted as described in section 7.1. The filters were probed with MAB specific for PKC type III (UBI). The lane to the right is loaded with 20 ng of purified rabbit brain PKC-α.

FIG. 18A-I. In vitro phosphorylation of PKC sites 1-4 on SSeCKS. 18A and 18B: Five μg of various GST-SSeCKS fusion proteins containing individual or combinations of the predicted PKC phosphorylation sites in SSeCKS, were subjected to an in vitro PKC assay containing [32P]-γ-ATP and analyzed as in FIG. 15. 18C-F and 18G-I: Expression and purification of the GST-SSeCKS fusion proteins. Fifty μg aliquots of bacterial lysate from uninduced (lane a) or induced (lane b) bacteria, or 5 μg of GST-SSeCKS fusion protein eluted from glutathione-Sepharose columns (lane c), were analyzed by SDS-PAGE, and then stained with coomassie blue. Arrows indicate the size of the unfragmented protein product.

FIG. 19. SSeCKS is resistant to heat denaturation. 150 μg aliquots of Rat-6 cell lysate were boiled for 5 min in the absence of SDS and then debris removed by low speed centrifugation (1K rpm at 4° C.). Lane c represents supernatant which was applied directly onto an SDS/poly-acrylamide (5%) gel, whereas lane b was boiled supernatant first immunoprecipitated with rabbit anti-SSeCKS serum (lane b) as described in section 7.1 (IgH is immunoglobulin heavy chain recognized by the AP-labeled sheep anti-rabbit Ig secondary antibody). Lane a contains the SSeCKS protein remaining in the lysate after the immunoprecipitation in lane b. Note that under these conditions, >95% of the SSeCKS protein is usually immunoprecipitated. Lane d contains 150 μg of unboiled lysate run directly on the gel.

FIG. 20. SSeCKS expression in src- and ras-transformed cells. 250 μg of total protein from Rat-6, Rat-6/src and Rat-6/ras (1) cell lysates was analyzed by immunoblotting for SSeCKS as described in section 7.1. In addition to the 240 kDa (larger arrow) and 280/190 kDaa (small arrow) forms of SSeCKS found in untransformed cells, a 305 kDa form was detected in Rat-6/ras cells and to a lesser extent in Rat-6/src cells. The relative level of SSeCKS in src- and ras-transformed cells compared with Rat-6 cells (as defined by densitometry) is 15- and 8-fold lower, respectively.

FIG. 21A-J. Immunofluorescence analysis of SSeCKS cellular localization. Subconfluent (G-J) or confluent (A-F) cultures of 3Y1 rat fibroblasts were fixed and analyzed for SSeCKS (A, C, E, H-J) or actin (B, D, F) expression as described in section 7.1. Panel G represents cells incubated with pre-immune rabbit sera. SSeCKS was pre-sent throughout the cytoplasm in subconfluent and confluent cells (e.g.—panel J, “cy”; panel A) and in the paranucleus (e.g.—panel J, “pn”). SSeCKS was also enriched in focal contact sites (e.g.—panel H, arrows), in podosomes (e.g.—panel I, “p”) and at the cell edge (panel J, “ce”; panel A). Confluent 3Y1 cells showed mostly cytoplasmic staining of SSeCKS (A), possibly associated with cortical actin but not with actin stress fibers (B). After 10 min treatment with 200 nM PMA, SSeCKS moved away from plasma membrane sites towards the paranucleus (C), simultaneous with the ruffling of actin fibers at the membrane (D). The inward movement of SSeCKS and the ruffling of actin became more pronounced after 60 min treatment with PMA (E and F, respectively).

FIG. 22. SSeCKS does not enter a soluble cellular component after short-term PMA treatment. Con-fluent Rat-6 or Rat-6/PKC-α overexpressor cell cultures were grown over-night in DEM lacking calf serum, and then treated with PMA (1.6 μM) for 30 min or mock-treated for the same duration. The cells were lysed and spun at low speed (1.5K rpm), yielding a pellet (P1) and supernatant (S1). The S1 component was fractionated by differential centrifugation into plasma membrane (P100) and soluble (S100) subcellular components as described in section 7.1. 50 μg aliquots of P1 and P100, and 25 μg aliquots of S100 were then immunoblotted using rabbit anti-SSeCKS as in FIG. 14. The SSeCKS isomers (240, 280, and 290 kDa) are shown in relation to a 220 kDa marker protein (myosin heavy chain).

FIG. 23A. Identification of a human SSeCKS gene homologue. 2 μg of poly A+ mRNA from various tissues was probed with radiolabeled rat SSeCKS cDNA under conditions of stringent hybridization. The tissue distribution and message size in humans is similar to that in mice (FIG. 9).

FIG. 23B. Western blot of SSeCKS expression in various mouse tissues and in human fibroblasts (WI-38) using antibody directed against rat SSeCKS protein. Note that anti-SSeCKS sera recognizes a 280/290 kDa doublet in human cells. Taken with the data in FIG. 23A, this indicates that humans encode an SSeCKS homologue.

FIG. 24. Northern blot of RNA prepared from various human tumor cell lines, using radiolabeled rat SSeCKS cDNA as a probe.

FIGS. 25A and B. Full-length SSeCKS decreases v-src-induced colony formation in soft agar.

FIG. 26. Amino acid sequences associated with myristylation and palmitylation (SEQ ID NOS:5-10).

FIG. 27. Inhibition of proliferation of cells in tetracycline-containing (+) and tetracycline-free (−) media by SSeCKS, encoded by a tetracycline-repressed construct and expressed in the absence of tetracycline.

FIG. 28. Polyacrylamide gel electrophoresis (PAGE) showing labeling of SSeCKS, encoded by a tetracycline-repressed construct and expressed in the absence of tetracycline, with tritiated myristate.

FIG. 29. Northern blot showing expression of SSeCKS RNA in the testes of normal Swiss mice and weaver mutant mice.

FIG. 30A-D. Photomicrographs showing S24 cells transfected with tetracycline-repressed SSeCKS construct in tetracycline-free (30A and 30B) and tetracycline-containing medium (30C and 30D).

FIG. 31A-D. Photomicrographs of tetracycline-repressed SSeCKS transfected S24 cells stained with fluorescent labeled antibodies to SSeCKS (31A and 31C) and F-actin (31B and 31D) in the presence (31A and 31B) and absence (31C and 31D) of tetracycline.

FIG. 32A-H. Overexpression of SSeCKS delays the formation of vinculin-associated adhesion plaques (ap). S24 cells (see FIG. 1) grown in the absence of tetracycline for 1 (a,b,g,h), 3(c,d) and 4 (e,f) days were stained for SSeCKS (a,c,e and g) and vinculin (b,d,f and h). After 1 day, adhesion plaques were detected only in the cell not over-expressing SSeCKS (left cell, panel a/b). After 3 days, adhesion plaques began to form in the SSeCKS overexpressor cells but were not located at the cells' leading edges (1e). After 4 days, adhesion plaques were detected at the leading edge in the SSeCKS overexpressor cells. Panels g and h show the inverse expression pattern of SSeCKS and vinculin in filopodia of overexpressor (S24) and non-overexpressor cells (n).

FIG. 33A-H. Photomicrographs depicting fluorescent staining with anti-actin antibodies (33B, 33D, 33F, and 33H) or anti-SSeCKS antibodies (33A, 33C, 33E, and 33G) in cell-wounding experiments.

FIG. 34. SSeKCS transcriptional down-regulation correlates with anchorage-independent growth. A northern blot containing 15 μg per lane of total RNA from a panel of ras-transformed or revertant cells described in (Feinleib and Krauss, 1996. Molec. Carcinog. 16:139-148) was probed with [³²P]-labeled SseCKS cDNA. PKC-β overexpressors and PKC-β/ras cells represent untransformed and fully transformed controls, respectively. ER1-2 cells are flat untransformed revertants of PKC-β/ras, and ER1-2T are partially transformed revertants of ER1-2 that grow in soft agar (not as many colonies as produced by PKC-β/ras cells). ER1-2/ras are ER1-2 cells transduced with activated ras, which exhibit somewhat increased refractility (morphological transformation) but not growth in soft agar. Note that suppression of SSeCKS transcription is detected in ER1-2T cells but not in ER1-2/ras.

FIG. 35. Suppression of src-induced soft agar colonies in a transient expression assay. NIH3T3 cells were transfected with either pMv-src or pMv-src plus pBABE/SSeCKS (equal molar gene equivalents), and the cells were either incubated in soft agar suspension supplemented with hygromycin for colony formation (panel A) or plated onto dishes without selection for focus formation, as described in Materials and Methods. The number of cells plated in each experiment is shown above each set of columns.

FIG. 36A-B. (A) Effect of v-src and SSeCKS expression on cell morphology and transformation. S24/ts72src cells were grown to confluence at either the PT (35° C.) or NPT (39.5° C.) for src kinase activity, in the presence (1 μg/ml) or absence of tetracycline. Note the induction of morphological transformation in presence of tetracycline at the PT only. Additionally, note the increased cell fattening in the absence of tetracycline, especially at the NPT. (B) Immunofluorescence analysis of SseCKS expression in S24/ts72src cells. S24/ts72 src cells were grown on coverslips at either the PT or NPT in the presence or absence of tetracycline, and then fixed and stained for SSeCKS using immunoaffinity-purified sera, as described in Materials and Methods. Note the extreme cell flattening at the NPT without tetracycline, and fattening to a lesser extent at the PT without tetracycline. Filopodia- and lamellipodia-like structures were found at either temperature in the absence of tetracycline.

FIG. 37A-B. Overexpression of SSeCKS suppresses anchorage-independent growth of S24/ts72src cells. 10⁵ S24/ts72src cells were grown in soft agar suspension at either the PT or NPT in the presence or absence of tetracycline for 3 weeks, at which point colony sizes (A) and numbers (B) were scored. The colonies formed at the PT without tetracycline were uniformly smaller than those formed in the presence of tetracycline.

FIG. 38A-C. Re-expression of SSeCKS to “normal” levels suppresses anchorage-independent growth of S24/ts72src cells. (A) Immunoblot analysis showing the effect of varying tetracycline concentration on expressed levels of SSeCKS in S24/ts72src cells at the PT. Note that the levels of endogeneous SSeCKS are suppressed at the PT compared with parental NIH3T3 cells. Using 0.02 μg/ml of tetracycline in the soft agar suspension, the number (B) and size (C) of the resulting colonies were scored. Panel C shows that the colonies forming in 0.02 μg/ml of tetracycline were typically small than those formed in 0. μg/ml of tetracycline.

FIG. 39A-B. Effect of SSeCKS expression on the proliferation rates S24/ts72src clones. 5×10⁴ cells were seeded per well and allowed to proliferate at the PT in DMEM supplemented with 10% (A) or 0.5% (B) heat-inactivated calf serum. Removal of tetracycline caused a roughly 40% decrease in the proliferation rate of control cells (pLJ), most probably due to toxicity associated with high expression of the tTA (Tetr/VP16) transactivator, as described in Gossen and Bujard, 1992, Proc. Nat'l. Acad. Sci. 89:5547-5551 and Shockett et al., 1995, Proc. Natl. Acad. Sci U.S.A. 92:6522-6526. Although the initial proliferation rates of the S24/ts72src clones are similar in the presence (w) or absence (w/o) tetracycline, those grown without tetracycline reach higher saturation densities, as shown in FIG. 36A.

FIG. 40A-D. Effect of SSeCKS expression on v-src expression and activity in S24/ts72src clones. (A) Immunoblot of SSeCKS protein levels in the S24/ts72src clones at the PT in the presence (+) or absence (−) of tetracycline. Note (i) the suppressed levels of endogenous SSeCKS in the S24/ts72src clones compared with the control clones (pLJ) in the presence of tetracycline, and (ii) a >20-fold increase in SSeCKS levels after the removal of tetracycline. (B) Immunoblot of ts72src protein in the S24/ts72src or control clones at the PT in the presence (+) or absence (−) of tetracycline, using Mab-EC10 which recognizes avian (i.e.—exogenous) p60src only (Parsons et al., 1984, J. Virol. 51:272-282). (C) In vitro ts72src kinase activity from S24/ts72src or control clones grown at the PT in the presence (+) or absence (−) of tetracycline, detected as transphosphorylation of immunoglobulin heavy chain in a src autophosphorylation assay. (D) in vivo ts72src kinase activity from S24/ts72src or control clones grown at the PT in the presence (+) or absence (−) of tetracycline, detected by anti-phosphotyrosine immunoblot with Mab-PY20.

FIG. 41A-C. Effect of SSecks expression on v-src-induced ERK2 and JNK protein levels and kinase activities. The levels of ERK2 protein (A) and activity (B) isolated from S24/ts72src or control clones grown at the PT in the presence (+) or absence (−) of tetracycline were determined. The level of JNK activity (C) isolated from S24/ts72src or control clones grown at the PT in the presence (+) or absence (−) of tetracycline was determined on either GST-JUN or GST control protein.

FIG. 42A-B. Effect of SSeCKS expression on v-src-induced changes to vinculin-associated adhesion plaques or actin-based stress fibers. S24/ts72src cells were grown on coverslips and processed as in FIG. 36B with the addition of either mouse monoclonal antivinculin antibodies followed by rhodamine-labeled anti-mouse Ig (A) or rhodamine-labeled phalloidin (B). In panel A, note that the transformed cells with tetracycline have fewer but larger vinculin-associated adhesion plaques compared to untransformed cells with tetracycline. Although SSeCKS overexpression causes an initial loss of typical adhesion plaques at the NPT, these structures are apparent at the cell's leading edge at the PT as is seen in the untransformed cells in the presence of tetracycline. Similar effects on stress fiber formation are detected in panel B, with the caveat that SSeCKS induces smaller fibers at the PT in comparison to untransformed cells with tetracycline.

FIG. 43. Mapping of Gravin/AKAP12 to chromosome 6q24-25.2. FISH analysis was performed on metaphase chromosomes of human peripheral blood lymphocytes using a FITC-labeled Gravin cDNA. The chromosomes were counterstained with DAPI, and the FITC and DAPI images were overlapped to identify specific chromosomes. Note the lone gravin signal (allele pair) at 6q. Of 100 chromosome sets analyzed, 81% showed hybridization signals, and 10/10 randomly chosen sets showed FITC signals corresponding to region 6q24-25.2 (dots next to right panel).

FIG. 44A-D. Loss of SSeCKS/Gravin expression in prostate cancer. (A) Northern blot analysis of total RNA (25 μg/lane) from Dunning rat prostate cancer cell lines grown in culture or in Copenhagen rats, probed with [³²P]-rat SSeCKS cDNA (SSeCKS message is 6.3 Kb). 28S rRNA, stained with ethidium bromide, is the loading control. The R3227-G cell line, derived from the original R3227 tumor, is strictly androgen-dependent and displays poor transformed growth potential in vitro. The R3327-H tumor has cells with mixed tumorigenic potential; Mat-Ly-Lu and Mat-Lu were derived clonally from AT-1, an isolated tumorigenic clone from R3327-H, and AT-3 is a highly-metastatic, anaplastic clone derived from lines grown in castrated Copenhagen rats. (B) Northern blot analysis of poly A+ RNA (2 μg/lane) from human prostate cancer cell lines or total normal human prostate, probed at high stringency with [³²P]-rat SSeCKS cDNA (Gravin/AKAP12 message is 6.5 Kb; “NS”—non-specific signal; upper band with triangle identifies an aberrant ˜8.5 Kb Gravin message in PC-3 and PPC-1 cells). “LNCaP—androgen”: two LNCaP cells lines grown without androgens for >9 months. The blot was stripped and reprobed for actin as a loading control (below). (C) Western blot of protein (50 μg/lane) from various rodent or human prostate cell lines, probed with polyclonal anti-SSeCKS Ig. The blot was stained with Amido Black (BioRad) to control for protein loading. Confluency: L=low (<40%), M=medium (70-80%), H=high (saturation density). SSeCKS isoforms are identified by arrows relative to protein mol wt markers (290/280 kD=doublet arrow; 240 kD=triangle). Densitometry indicated that the relative levels of SSeCKS in EP12 was 4- and 10-fold higher in M and H cultures, respectively, relative to L cultures, whereas the relative SSeCKS levels in MLL M and H cultures was 1.5-fold lower than in L cultures. After stripping, the blot was reprobed with anti-vinculin as a loading control. (D) High culture density of MLL does not alter the relative abundance of the 290/280 kD SSeCKS isoforms but increases the abundance of the ˜80 kD isoform. Lysates were probed with anti-actin as a loading control. The relative abundance of SSeCKS/Gravin protein levels was determined by densitometry.

FIG. 45A-B. Production of MLL cell lines with tet-regulated SSeCKS expression. (A) 50 μg of protein from MLL/tTAK, EP12, MLL, or MLL/SSeCKS clones 2-6, 2-7, and 7-2 were immunoblotted for SSeCKS. The 290 and ˜80 kD SSeCKS isoforms are identified by arrows (right) relative to protein markers (left). Lysates were probed with anti-actin as a loading control. (B) Re-expression of SSeCKS suppresses morphological transformation and increases cell-cell adhesion. Phase contrast microscopy of MLL[tet/vector] (V-2) or MLL[tet/SSeCKS] clones (2-4, 2-6, and 2-7) grown in the presence or absence of tet for 3 d. Note the decreased refractility and increased cell flattening in the MLL[tet/SSeCKS] clones grown without tet. All magnifications are 100×.

FIG. 46A-B. IFA analysis of SSeCKS and F-actin. (A) EP12 cultures fixed in acetone/formaldehyde were stained for SSeCKS (a,b) or F-actin (a′/b′) (TRITC-phalloidin). Note the cortical cytoskeletal SSeCKS staining marked by enrichments in the perinuclear region, in membrane ruffles (panel a; arrows), in focal contacts at the lagging edge (panel b; triangle) and in actin-rich concentrations at the leading edge (panel b; arrows). (B) Re-expression of SSeCKS in MLL[tet/SSeCKS] cells grown without tet (panels a and b) shows enrichment of SSeCKS in membrane ruffles (panel a) and in lamellipodia (panel b). EP12 (panel c), showing typical cell-cell adhesion and epithelial morphology, is shown for comparison. In contrast, little endogenous SSeCKS staining is detected in MLL[tet/vector] cells grown with (panel d) or without (panel e) tet, or MLL[tet/SSeCKS] cells grown with tet (panel f). Magnification for all panels is 630×; size bars are 20 nm.

FIG. 47A-C. Ectopic SSeCKS decreases MLL proliferation rate and anchorage-independence. (A) MLL[tet/vector] control (V-2, V-7) or MLL[tet/SSeCKS] clones were grown in media containing 10% FCS with (solid lines) or without tet (broken lines) and then monitored for proliferation. All experiments were performed in triplicate, and variations at any time point for a given sample were less than 15%. (B) Triplicate aliquots of 10⁴ MLL[tet/vector] or MLL[tet/SSeCKS] cells were grown in soft agar overlays for 3 weeks at which point the number of colonies formed was determined. In the absence of tet, control cells formed ˜40% fewer colonies than if grown with tet. In contrast, the MLL[tet/SSeCKS] clones produced 8-12 fold fewer colonies in the absence of tet. Several SSeCKS-expressor soft-agar colonies were isolated and grown in culture, and all showed a loss of tet-inducible SSeCKS expression. (C) SSeCKS-induced suppression of anchorage-independence in the absence of tTAK. Triplicate aliquots of MLL cells were transiently transfected with a 1:4 ratio of pBABEhygro and either pcDNA3 or pcDNA/SSeCKS, and then grown for three weeks in soft agar supplemented with hygromycin. Transfection efficiencies were normalized by co-transfecting 1.5 μg of pEGFP and monitoring cell fluorescence.

FIG. 48A-D. Effect of ectopic SSeCKS expression on tumor growth in nude mice. (A) Athymic nu/nu mice (6/experiment) were injected s.c. with 10⁵ MLL[tet/vector] or MLL[tet/SSeCKS] cells. All mice were maintained on tet/sucrose-water (changed every 2 days). When primary tumors were palpable, half the mice were switched to plain water (−tet) whereas as the others continued with tet-water (+tet). The average tumor volumes at primary injection sites are shown and the error bars represent the range of tumor volumes in a given cohort. This experiment was repeated twice with two MLL[tet/vector] and two MLL[tet/SSeCKS] clones. The X axis represents the days following initial tumor palpation. (B) Relative tumor burden at the primary injection site (O) compared to lung metastases (●). Results compare two independent experiments of MLL[tet/vector] (clones V-2 and V-7) in mice receiving no tet to “SSeCKS”, MLL[tet/SSeCKS] clones 2-6 and 7-2, in mice receiving tet (+tet) or regular water (−tet). (C) 18-day tumors from mice in the experiment in Panel A were excised and analyzed by western blotting for SSeCKS. Note the loss of tet-regulated SSeCKS expression in MLL[tet/SSeCKS] clone 2-6 from mice initially fed tet and then fed water lacking tet (±), compared to 2-6 cells grown in culture. (D) Monolayer wounding assay showing no effect of SSeCKS expression on cell motility (clone 2-6) compared to vector (V-2) controls.

FIG. 49. Loss of SSeCKS/Gravin in advanced human prostate cancer in situ. Formalin-fixed sections of human prostate representing normal tissue (a) showing enriched Gravin staining in basal epithelial cells (B), benign prostatic hyperplasia (b), well differentiated prostate cancer (c) and advanced, undifferentiated prostate cancer in situ (d) were processed and stained by immunohistochemistry for SSeCKS/Gravin protein. Panel d is shown at a lower magnification to demonstrate the loss of Gravin staining in a high-grade lesion adjacent to abundant staining in unaffected epithelium. Size bars equal 10 nm.

FIG. 50A-D. SSeCKS overexpression results in growth arrest. (A) Western blot showing expression of SSeCKS in tet-regulated clones. Cell lysates of tet/SSeCKS clones (S24 and S33) and vector control clones (V3 and V4) grown in the presence or absence of tet were used for western blotting analysis normalized for 35 μg protein per lane. In the absence of tet, ectopic expression of the 290 kDa SSeCKS isoform was induced in S24 and S33 cells. (B) Morphological changes after SSeCKS expression. S24 and V3 cells grown in the presence or absence of tet were immunostained for SSeCKS expression. SSeCKS overexpression results in cell flattening and production of filopodia- (F) and lamellipodia- (L) like projections. Removal of tet did not affect the morphology of control V3 cells. Magnification is 250×. (C) SSeCKS induces growth arrest. The proliferation rate of S24 cells is dramatically reduced when SSeCKS is overexpressed. The reduced proliferation of V4 cells is most likely due to squelching effects of the tTA transactivator. (D) SSeCKS-induced growth arrest is reversible. Tet (0.5 μg/ml) was added back to cell cultures at day 6 after its removal. Two days later, coincident with the degradation of ectopic SSeCKS, S24 cells were proliferating exponentially, indicating that SSeCKS overexpression does not induce cellular senescence.

FIG. 51A-B. SSeCKS overexpression results in the loss of cyclin D1 expression and pRb hypophosphorylation. S24 and V3 cells were grown in the presence or absence of tet for 4 days before collecting cell lysates. (A) Western blot analysis showing the steady state levels of cyclins, CDKs, and CKIs. Among the examined cell cycle regulators, only the level of cyclin D1 is specifically reduced in response to SSeCKS overexpression. The relative protein levels between lanes were normalized by loading equal aliquots of protein and by stripping and reprobing the same blot. These results were confirmed at least twice more. (B) Western blot showing the phosphorylation status of pRb. The relative abundance of hypophosphorylated (“hypo”; faster migrating band) pRb is greatly increased when SSeCKS expression is induced.

FIG. 52A-C. SSeCKS overexpression inhibits cyclin D transcription and serum-inducible ERK2 activity. S24 cells grown in the presence or absence of tet were starved of serum overnight, then stimulated with media supplemented with 10% calf serum for various periods. (A) ERK2 levels detected by western blotting analysis. (B) ERK2 kinase activity measured by in vitro phosphorylation of myelin basic protein (MBP) substrate. SSeCKS overexpression results in a >5 fold decrease in serum-inducible ERK2 kinase activity and a decrease in the length of EKR2 activation. (C) Northern blot analysis of total RNA (25 μg/lane) from S24, V3, S24/D1 and S24/V cells lines grown in the presence or absence of tet, probed with [³²P]-labeled cyclin D1 cDNA. Note the decreased accumulation of cyclin D1 RNA in S24 and S24/V cells when SSeCKS is re-expressed, in contrast to V3 and S24/D1 cells in which cyclin D1 levels do not decrease in the absence of tet. The S24/D1 lanes are shown at right with a shorter exposure to facilitate comparison. RNA levels were normalized to rRNA levels shown at bottom.

FIG. 53A-E. Ectopic expression of cyclin D1 fails to rescue SSeCKS-induced G1 arrest. (A) Western blot analysis (150 μg total protein/lane) showing ectopic expression of cyclin D1 in S24 cells (S24/D1) achieved by retroviral transduction, followed by G418 selection for stable clones. S24/V are vector-infected S24 cells. (B) Morphology of S24/D1 cells. S24/D1 cells grown in the presence or absence of tet were immunostained for SSeCKS expression. Ectopic expression of D1 fails to revert SSeCKS-induced cell flattening. Magnification is 1000×. (C) SSeCKS overexpression inhibits the proliferation of S24/D1 cells to a similar extent as with S24/V cells. (D) Cell cycle analysis as described in Materials and Methods. SSeCKS overexpression results in an increased abundance of S24/D1 cells in G1 phase. (E) Western blot analysis showing the mobility status of pRb on SDS-PAGE. The majority of pRb in S24/D1 and S24/V cells is hypophosphorylated (“hypo”) in response to SSeCKS expression.

FIG. 54. Sequence similarity between SSeCKS and the Abl-binding domain in pRb. Identical a.a. residues (vertical lines) or similarly charged residues (colons) are shown for the SSeCKS and newt Rb (Genbank accession # Y09226) proteins.

FIG. 55A-C. SSeCKS overexpression redirects cyclin D1 to the cytoplasm. (A) Confocal analysis of cyclin D1 immunostaining. S24/D1 and V3/D1 (V3 cells overexpressing D1) cells grown in the presence or absence of tet were fixed and immunostained using PAb anti-cyclin D1. The nuclear staining of cyclin D1 is diminished and cytoplasmic staining increased in response to SSeCKS overexpression. Note that the total cyclin D1 protein level is unchanged by tet in S24/D1. 4A. Magnification is 100×. (B) Confocal images of SSeCKS and cyclin D1 co-immunostaining. Cyclin D1 co-localizes in the cytoplasm with SSeCKS following SSeCKS overexpression. (C) Confocal images of SSeCKS and cyclin D1 double-immunostaining viewed from the X-Z axis. Magnification is 630×.

FIG. 56. Short-term activation of PKC induces the nuclear translocation of cyclin D1 sequestered in the cytoplasm. S24/D1 cells grown on coverslips in the presence or absence of tet were serum-starved overnight and treated for 30 min with 200 nM PMA or solvent (dimethylsulfoxide). The cells were fixed and stained for cyclin D1. Note the increase in nuclear cyclin D1 staining after PKC activation (typified by the exclusion of nucleolar compartments) in the absence of tet.

FIG. 57A-B. SSeCKS-induced growth arrest is not simply due to its overexpression at high levels. (A) Western blot showing the expression levels of SSeCKS and cyclin D1. S24/D1 and S24/V cells grown in the presence of 0.5, 0.02 or 0 μg/ml of tet were lysed for western blotting analysis. 0.02 μg/ml of tet induces ectopic SSeCKS expression levels only 2-3 fold above those of endogenous SSeCKS as determined by densitometric comparison. (B) S24/D1 are less growth-arrested than S24/V cells as the level of ectopic SSeCKS are decreased (compare proliferation rates of S24/D1 at 0.5 vs. 0.02 μg/ml of tet).

FIG. 58A-B. Penetratin-linked peptides encoding the SSeCKS CY motifs induce nuclear translocation of cyclin D. (A) Entry of penetratin-linked peptides. S24/D1 cells grown on coverslips in the absence of tet were incubated for 2 h in DMEM containing 100 μg/ml biotinylated peptides, washed, fixed, and incubated with FITC-labeled avidin. In contrast to the penetratin-linked peptides (top panel: wt CY, K→S mutant, and phospho-Ser^{507/515}) which entered cells, the non-penetratin-linked biotinylated peptides (bottom panel) failed to enter cells. (B) S24/D1 cells grown on 22 mm² coverslips in the absence of tet were incubated for 4 h with either unlabeled (top panel) or biotinylated (middle and bottom panels) penetratin-linked wt CY, K→S or phospho-Ser^{507/515} peptides, washed, fixed and then stained for peptide with FITC-avidin (bottom panel) or for cyclin D1 with PAb and TRITC-labeled anti-rabbit Ig (top and middle panels). Note that the unlabeled penetratin peptides show similar effects on cyclin D compartmentalization as the biotinylated forms. Also note that although the phospho-Ser^{507/515} peptide fails to chase cyclin D into nuclei, it caused partial reduction in cell flattening. Size bars=1 nm.

FIG. 59A-D. In vivo cytoplasmic sequestration of cyclin D by SSeCKS via CY domains correlates with contact-inhibition. (A) Immunoblots of cell lysates from untransformed rat embryo fibroblasts grown pre- and post-confluency, probed for SSeCKS (arrows show the 290 and 280 kDa isoforms), cyclins D and E, Cdk4, and CKIs p21 and p27. Actively-dividing, subconfluent cultures (−2 days) were allowed to achieve saturation density (day 1 of confluence), and the following day, they were either mock-treated or supplemented twice daily for 2.5 more days with 100 μg/ml of SSeCKS wt-CY or mutant K→S CY peptide. (B) The relative level of each protein band in panel A was determined by densitometric scanning and is represented at right as nuclear (white boxes) and cytoplasmic (black boxes) fractions. Relative protein levels were controlled by stripping the cyclin D blot and then re-probing with the other Abs shown. The error bars reflect the composite of duplicate experiments. (C) Proliferation index of 2d confluent cultures mock-treated or treated with wt, K→S mutant, or P-Ser^{507/515} CY peptides. After peptide treatment, cells were trypsinized, stained with trypan blue and counted using a hemacytometer. Error bars represent standard deviation based on analysis of triplicate samples for each treatment regimen. (D) Phase-contrast microscopy of rat embryo fibroblasts treated as in panel A, showing increased saturation density of the cells treated with the wt CY peptide yet no increase in cell refractility. Magnification=400×.

FIG. 60. Generation of monoclonal antibodies immunospecific against SSeCKS protein. As indicated the monoclonal antibodies bind to SSeCKS protein.

### 5. DETAILED DESCRIPTION OF THE INVENTION

The present invention relates to SSeCKS genes and proteins. For purposes of clarity of description, and not by way of limitation, the detailed description of the invention is divided into the following subsections: (i) SSeCKS genes; (ii) SSeCKS proteins; (iii) additional SSeCKS molecules; and (iv) utilities.

### 5.1. SSeCKS Genes

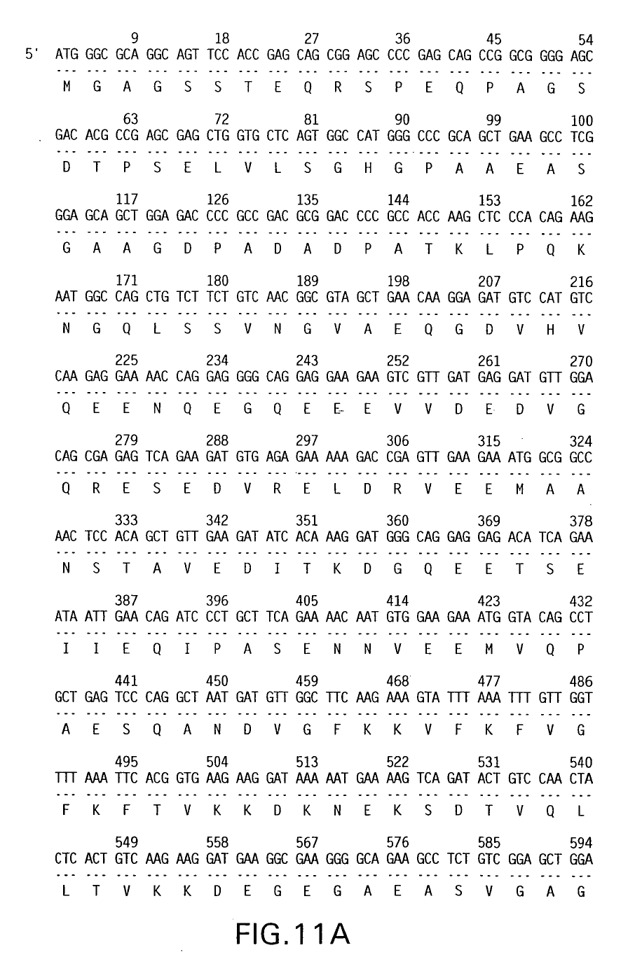

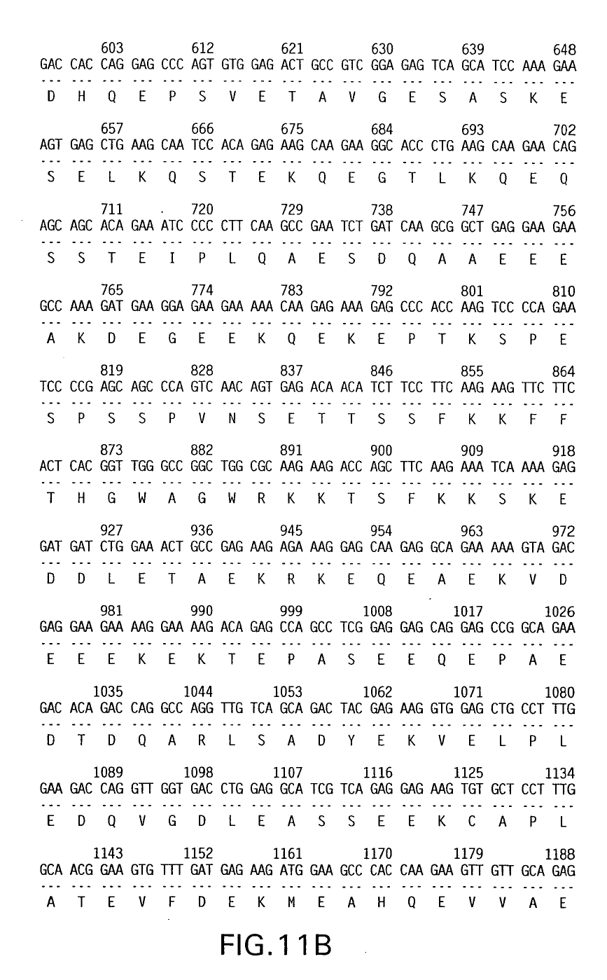

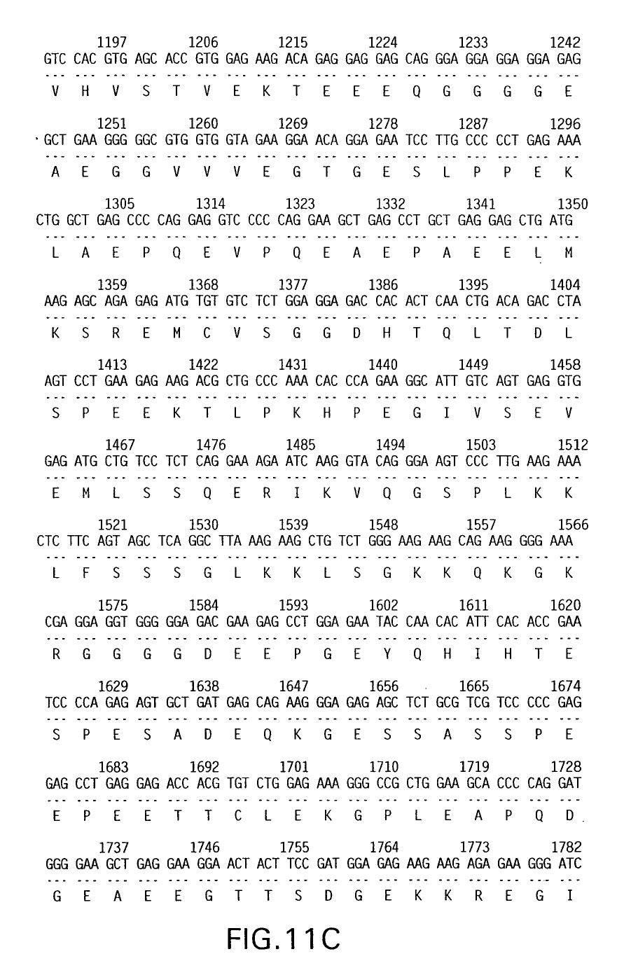

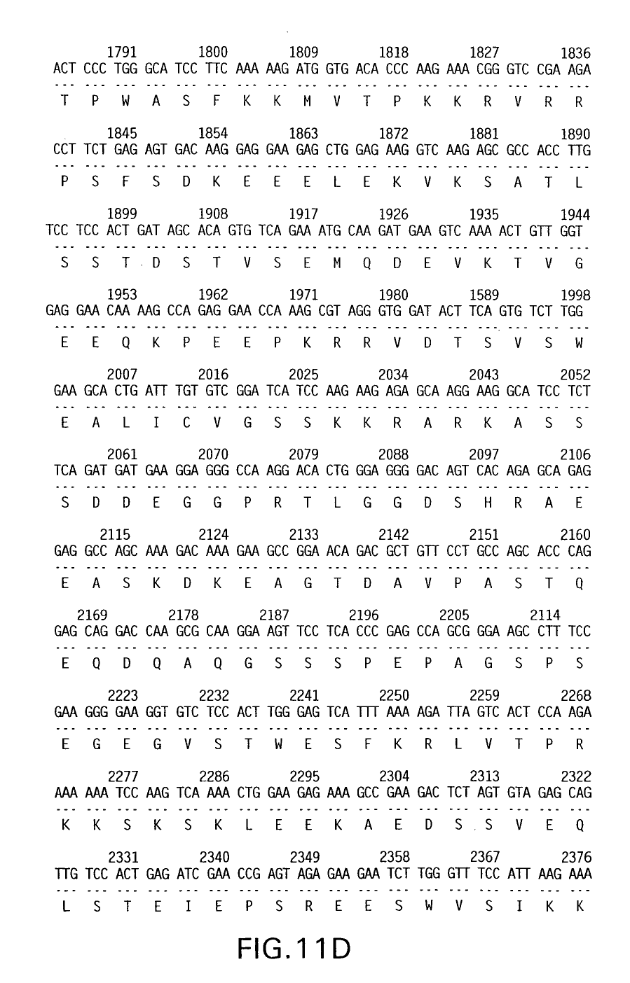

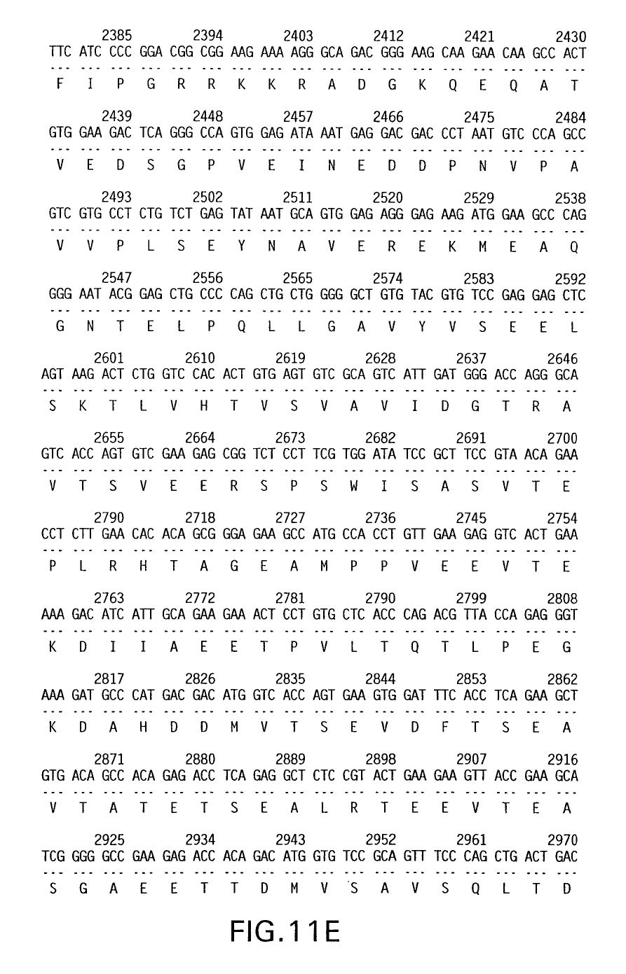

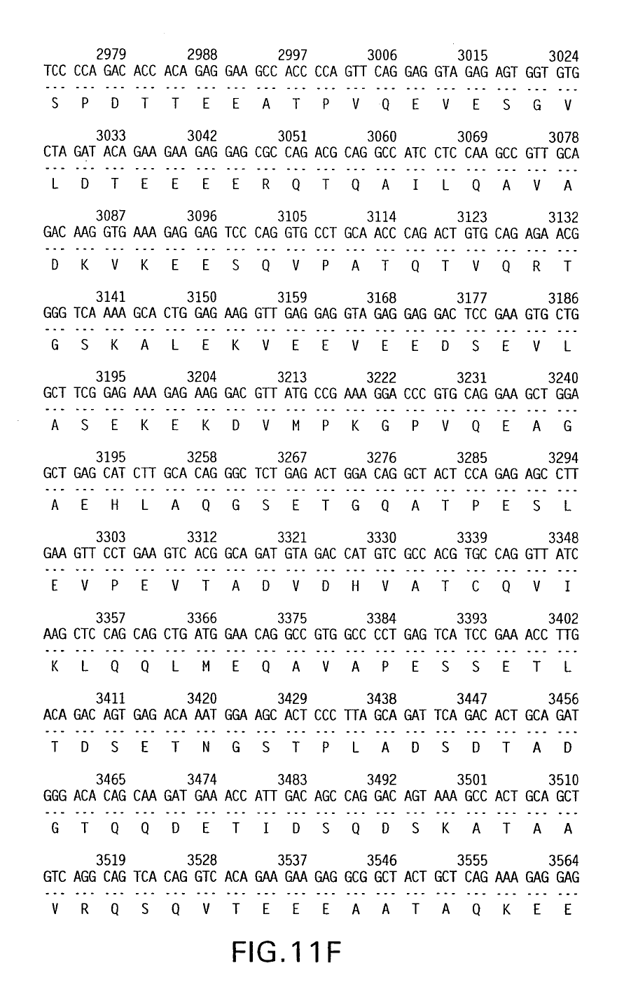

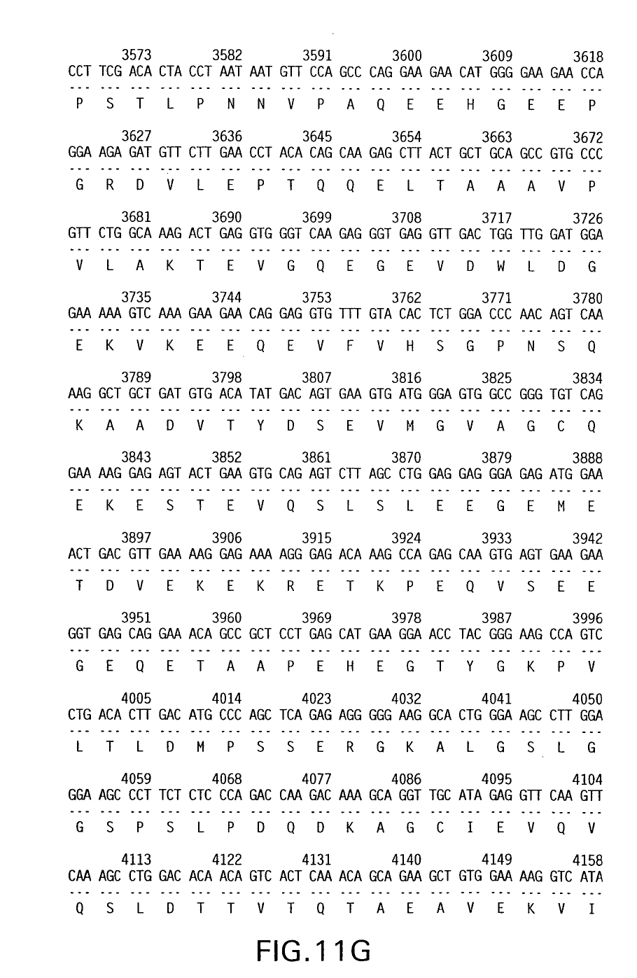

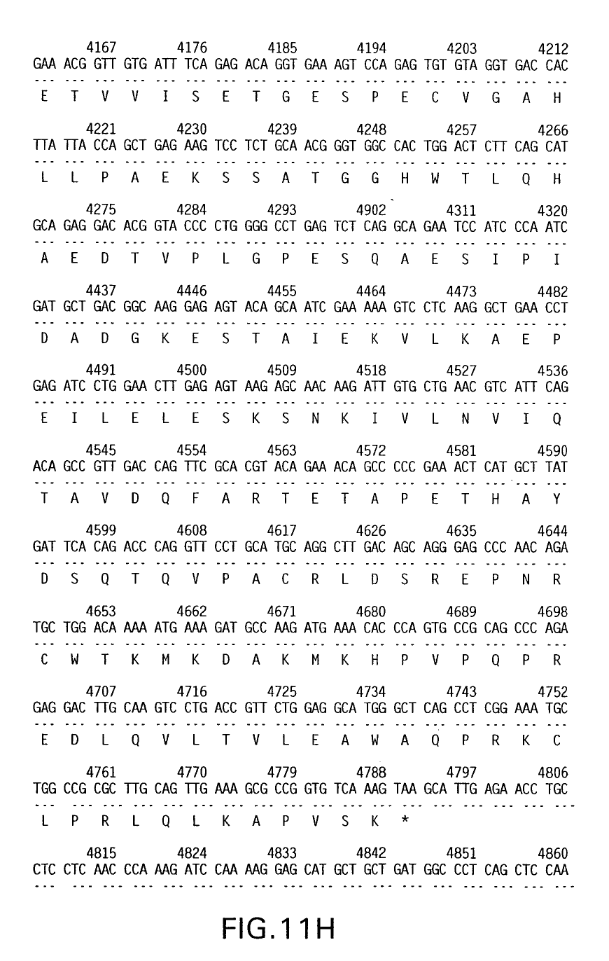

In one specific embodiment, the present invention relates to a purified and isolated nucleic acid molecule having the nucleic acid sequence set forth in FIG. 11 (SEQ ID NO:3), which is the full length rat SSeCKS cDNA. In another embodiment, the present invention relates to a purified and isolated nucleic acid molecule which hybridizes to a nucleic acid molecule having a sequence as set forth in FIG. 11 (SEQ ID NO:3) under stringent hybridization conditions. This embodiment would include nucleic acid molecules from species other than rat, such as the human SSeCKS cDNA. This embodiment would also relate to genomic DNA, RNA and antisense molecules. Stringent hybridization conditions are as described in Maniatis et al., 1982, in Molecular Cloning: A Laboratory Manual, Cold Spring Harbor Laboratory, Cold Spring Harbor, N.Y. In one specific, nonlimiting embodiment of the invention, stringent hybridization may be performed between DNA molecules in the Southern method, in a solution of 0.75 M sodium phosphate pH 7, 1 mM EDTA, 7% SDS, 1% bovine serum albumin (BSA), and 100 microgram per ml salmon sperm DNA for 12-18 hours at 65 degrees centigrade, followed by washing twice in 50 mM sodium phosphate, 1 mM EDTA, 1% SDS, and 0.5% BSA at 65 degrees C., and twice again in the same solution without BSA at 65 degrees centigrade.

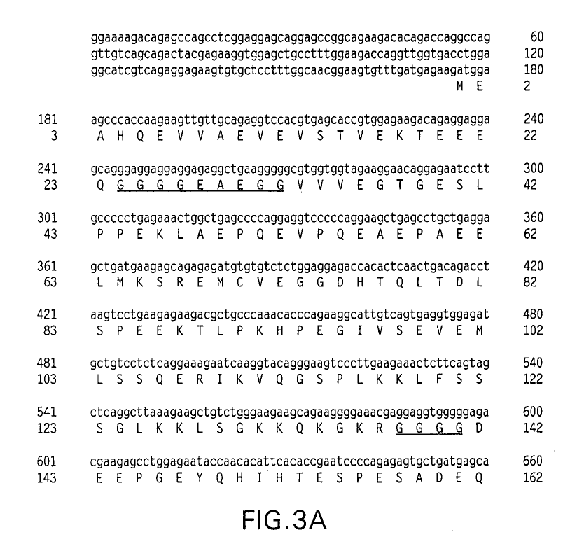

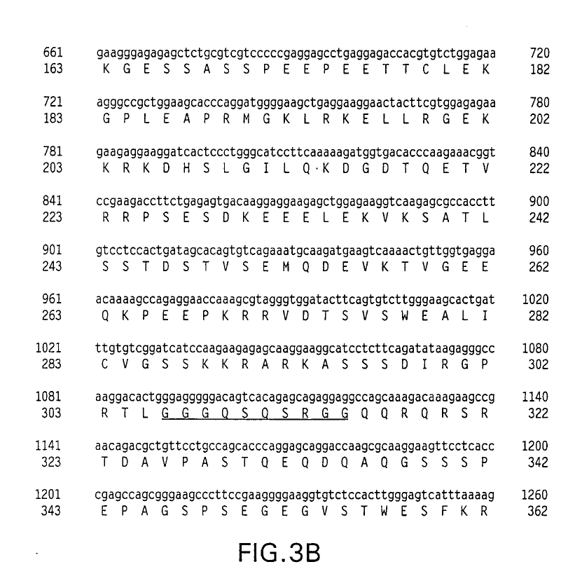

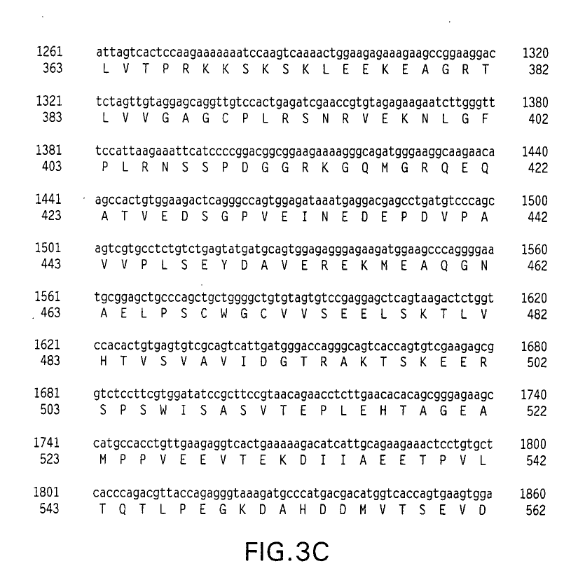

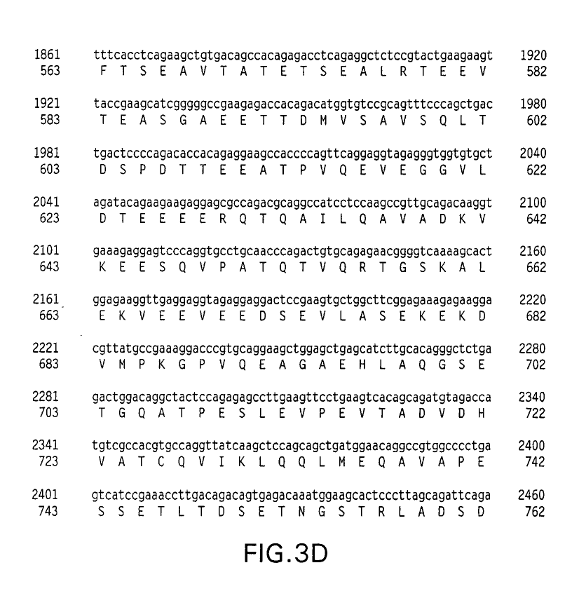

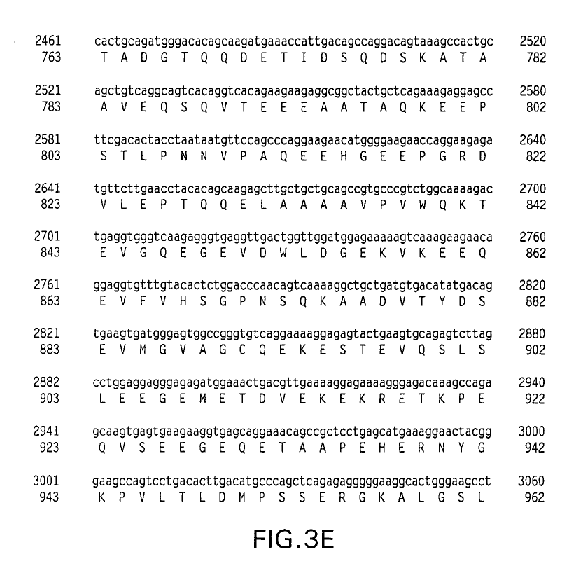

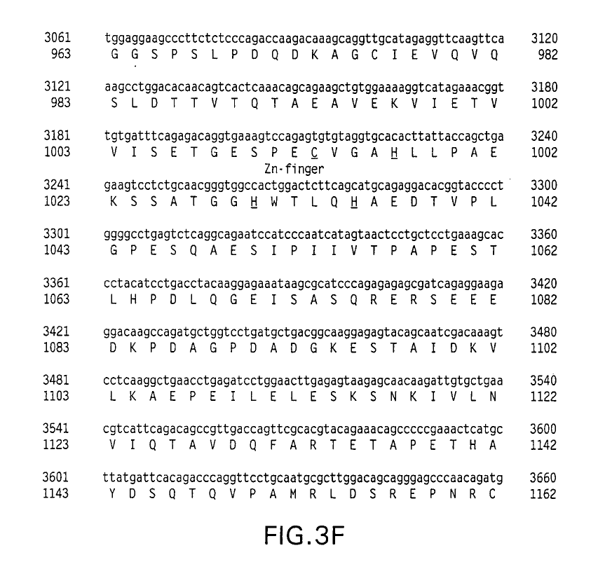

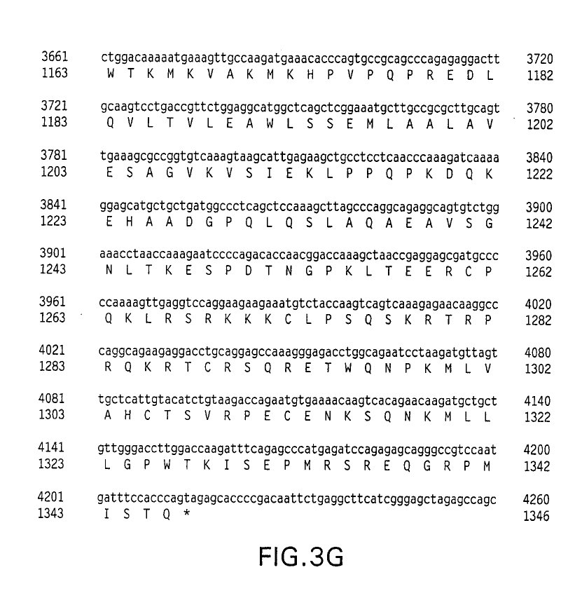

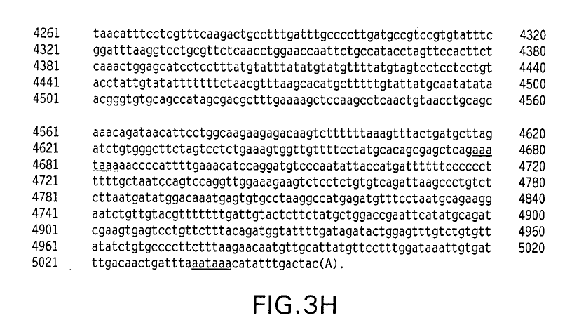

In yet another specific embodiment, the present invention relates to a purified and isolated nucleic acid molecule having the nucleic acid sequence set forth in FIG. 3 (SEQ ID NO:1), which is a truncated rat SSeCKS cDNA. In an additional embodiment, the present invention relates to a purified and isolated nucleic acid molecule which hybridizes to a nucleic acid molecule having a sequence as set forth in FIG. 3 (SEQ ID NO:1) under stringent hybridization conditions, as set forth above. This embodiment would include nucleic acid molecules from species other than rat, such as the human SSeCKS cDNA. This embodiment would also relate to genomic DNA and RNA molecules.

In related embodiments, the present invention provides for a purified and isolated nucleic acid sequence which is at least 90 percent homologous, and preferably at least 95 percent homologous, to either (i) a nucleic acid molecule having a sequence as set forth in FIG. 11 (SEQ ID NO:3) or (ii) a nucleic acid molecule having a sequence as set forth in FIG. 3 (SEQ ID NO:1). Homology may be determined using any standard software for calculating homology between nucleic acid molecules, for example, but not by way of limitation, the FASTA algorithm (Genetics Computer Group, Univ. Res. Park, Madison, Wis.; version 8.0)

The present invention also provides for nucleic acid molecules encoding either (i) a protein having an amino acid sequence as set forth in FIG. 11 (SEQ ID NO:4); or (ii) a protein having an amino acid sequence as set forth in FIG. 3 (SEQ ID NO:2).

### 5.2. SSeCKS Proteins

In further embodiments, the present invention provides for a purified and isolated protein having an amino acid sequence as set forth in FIG. 3 (SEQ ID NO:2) for truncated SSeCKS or in FIG. 11 (SEQ ID NO:4) for full length SSeCKS. In related embodiments, the present invention provides for functionally equivalent proteins. For example, one or more of the amino acid residues within the sequence may be substituted with another amino acid residue of a similar polarity which acts as a functional equivalent, resulting in a silent alteration. Substitutes for an amino acid within the sequence may be selected from other members of the class to which the amino acid belongs. For example, the nonpolar (hydrophobic) amino acids include alanine, leucine, isoleucine, valine, proline, phenylalanine, tryptophan, and methionine. The polar neutral amino acids include glycine, serine, threonine, cysteine, tyrosine, asparagine, and glutamine. The positively charged (basic) amino acids include arginine, lysine and histidine. The negatively charged (acidic) amino acids include aspartic acid and glutamic acid. Also within the scope of the invention are SSeCKS proteins that have been modified post-translationally, including, but not limited to myristylation, phosphorylation, glycosylation, and proteolytic cleavage, or by incorporation into a larger molecule.

The present invention also provides for a purified and isolated protein encoded by a nucleic acid molecule having the sequence set forth in FIG. 3 (SEQ ID NO:1) or (i) a nucleic acid molecule which hybridizes thereto under stringent conditions or (ii) is at least 90 percent, and preferably at least 95 percent, homologous thereto. The present invention still Esther provides for a purified and isolated protein encoded by a nucleic acid molecule having the sequence set forth in FIG. 11 (SEQ ID NO:3) or (i) a nucleic acid molecule which hybridizes thereto under stringent conditions or (ii) is at least 90 percent, and preferably at least 95 percent, homologous thereto. The present invention also provides for functional equivalents of these proteins, as defined above.

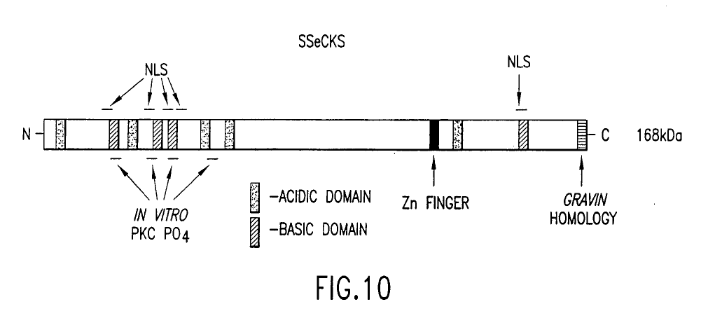

FIG. 10 depicts a schematic diagram of the SSeCK protein, which contains several sequence motifs consistent with a role of transcriptional regulator, including a putative Zn finger, at least five nuclear localization signals, and several highly acidic domains typical of transactivation factors such as GAL4.

### 5.3. Additional SSeCKS Molecules

The present invention provides for vectors comprising the above mentioned SSeCKS gene nucleic acid molecules, including plasmid, phage, cosmid, and viral vectors. The foregoing nucleic acid molecules may be combined, in such vectors or otherwise, with nucleic acid sequences which may aid in their expression, including promoter/enhancer sequences and other sequences which aid in transcription, translation, or processing. Vectors of the invention may further comprise other sequences, such as selection markers, as used by skilled artisans.

The present invention further provides for the isolated SSeCKS promoter, as may be identified in a genomic clone which hybridizes to the 5′ end of a nucleic acid molecule as depicted in FIG. 11 (SEQ ID NO:3). The precise location of the promoter may be analyzed by correlating the effect of site directed deletions in nucleic acid 5′ to the coding sequence with transcription of SSeCKS or a reporter gene. The SSeCKS promoter may be linked to a reporter gene and then used to study SSeCKS expression or the effects of various agents on SSeCKS expression. Because the promoter appears to be specifically inhibited in src- and ras-transformed cells, it may be used to indirectly study and identify agents that inhibit src-induced oncogenic transformation.

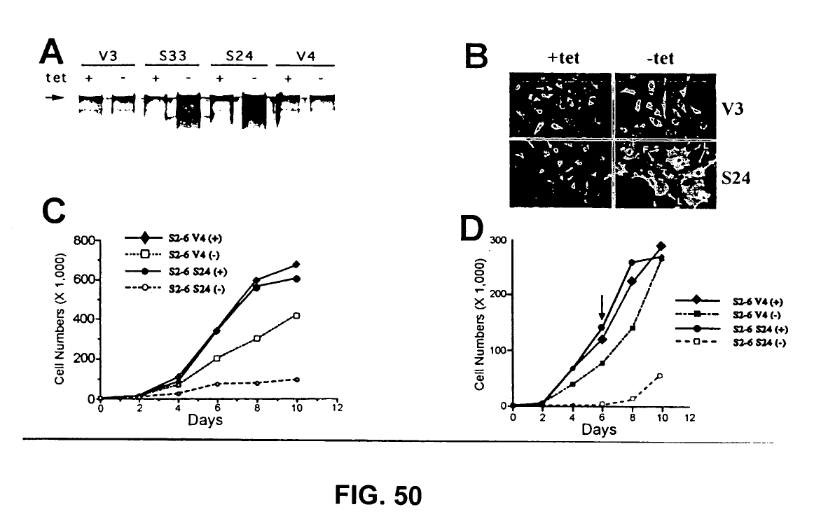

The present invention further provides for antibodies, including monoclonal or polyclonal antibodies, directed toward the proteins of the invention, and prepared by standard techniques known in the art. As described herein, monoclonal antibodies capable of binding to the SSeCKS protein (FIG. 50) were generated using routine methods, such as those described below. Monoclonal antibodies include those produced by the hybridoma cell lines designated 94A3 (ATCC NO.); 78H11(ATCC NO.); 82B3 (ATCC NO.); and 31A3 (ATCC NO.).

To improve the likelihood of producing an anti-SSeCKS immune response, the amino acid sequence of a SSeCKS protein may be analyzed in order to identify portions of the SSeCKS protein molecule which may be associated with greater immunogenicity. For example, the amino acid sequence may be subjected to computer analysis to identify surface epitopes, according to the method of Hopp and Woods, 1981, Proc. Natl. Acad. Sci. U.S.A. 78:3824-3828. Such epitopes may then be isolated and incorporated into a suitable carrier molecule.

For preparation of monoclonal antibodies toward a SSeCKS protein, any technique which provides for the production of antibody molecules by a continuous cell line or by an organism may be used. For example, and not by way of limitation, the hybridoma technique originally developed by Kohler and Milstein (1975, Nature 256:495-497), or the trioma technique (Kozbor et al., 1983, Immunology Today 4:72), or other techniques used for monoclonal antibody production, including methods for producing chimeric, humanized, or primatized antibodies, may be employed.

Alternatively, polyclonal antibodies directed toward a SSeCKS protein may be prepared by methods known in the art. Various adjuvants may be used to increase the immunological response, including but not limited to Freund's (complete and incomplete), mineral gels such as aluminum hydroxide, surface active substances such as lysolecithin, pluronic polyols, polyanions, peptides, oil emulsions, and keyhole limpet hemocyanin.

The present invention further provides for nucleic acids encoding immunoglobulin molecules directed toward a SSeCKS protein, including nucleic acids encoding single chain antibodies as well as conventional antibody molecules.

Antibody molecules may be purified by known techniques, such as immunoabsorption or immunoaffinity chromatography, chromatographic methods such as HPLC, or combinations thereof.

The present invention also provides for antibody fragments directed toward a SSeCKS protein, including, but not limited to F(ab′)2 and Fab fragments.

### 5.4. Utilities

The molecules of the present invention have a number of utilities. As described in the example section below, suppression of SSeCKS expression occurs in association with transformation by certain oncogenes or by the triggering of a proliferative cycle in starved cells by the addition of serum to the growth medium. These observations indicate that SSeCKS acts as a negative regulator of mitosis. As such, the introduction of SSeCKS gene or protein into a host cell may be used to inhibit mitosis of the host cell. Introduction may be achieved either via a vector, by physical means, or by direct uptake of the SSeCKS gene or protein into the host cell.

Moreover, it has been discovered that ectopic expression of SSeCKS suppressed the ability of v-src to induce morphological transformation and anchorage-independent growth in rodent fibroblasts. Thus, the introduction of SSeCKS gene or protein into a cell may be used to inhibit the expression of a transformed phenotype by the cell.

Since many human diseases are associated with disorders of proliferation and/or with the expression of a malignant (i.e. transformed) phenotype, increasing the levels of SSeCKS DNA, mRNA, and/or protein in a patient suffering from such a disease may be beneficial. For example, the levels of SSeCKS may be increased in a malignant tumor in such a patient in order to decrease its propensity to metastasize.

Furthermore, the level of SSeCKS expression in a cell or collection of cells may be used to evaluate the mitotic state of such cells, where a low level of SSeCKS expression may bear a positive correlation with active mitosis. Furthermore, a low level of SSeCKS expression may bear a positive correlation with a malignant phenotype. Such measurements may be used in the diagnosis or staging of tumorigenicity or malignancy, or in the assessment of the effects of therapeutic interventions in a subject in need of such treatment.

Because SSeCKS appears to be selectively expressed in testes and, to a lesser extent, brain, SSeCKS may be a marker for aberrancies in fertility and/or nervous system development. For example, decreased or absent expression of SSeCKS may be used as a marker for abnormal development of the testes or sperm in disorders of fertility.

In still further embodiments, the association between SSeCKS and cytoskeletal structures may be used to identify or treat disorders of cellular architecture. As an example, it is postulated that Alzheimer's Disease may result from defects in kinase-associated signal transduction pathways regulated by neuron-specific cellular architecture (Pelech, 1995, Neurobiol. Aging 16(3):247-256).

### 5.4.1 Assays for Measuring the Metastatic Potential of Cells

It is an object of the present invention to provide a method for the identification of subjects possessing a cancer with an increased metastatic potential. The present invention relates to the evaluation of metastatic potential by detecting a decrease in expression of SSeCKS within the subject's cancer cells. The present invention achieves a highly desirable objective, namely providing a method for the prognostic evaluation of subjects with cancer and the identification of subjects with a predisposition to developing metastatic cancer. Specifically, the invention encompasses assays for determining the metastatic potential of cancer cells isolated from a subject using assays designed to detect the level of SSeCKS expression within the cancer cells. The invention is based on the discovery that loss of SSeCKS expression correlates with the onset of cancer metastasis.

Specifically, the invention encompasses a method for determining the metastatic potential of cancer cells derived from a cancer subject comprising detecting the level of expression of SSeCKS in the cancer cells wherein a decrease in SSeCKS expression indicates the presence of cancer cells with increased metastatic potential.

Detection of SSeCKS expression may be achieved using a variety of different methods. For example, detection of SSeCKS RNA may be accomplished using, for example, Northern blot analysis, polymerase chain reactions, or in situ hybridizations. In addition, loss of the SSeCKS gene from the genome of the cancer cells can be detected using Southern blot analysis, FISH analysis, or polymerase chain reactions to name but a few. Finally, the level of SSeCKS proteins expressed within a cancer cell may be detected using a variety of different immunoassays, including but not limited to techniques such as Western blots, radioimmunoaasays, ELISA, sandwich assays, immunoprecipitation assays, and fluorescent immunoassays.

In addition, the assay system of the invention can also be used to monitor the efficacy of potential anti-cancer agents during treatment. For example, the metastatic potential of cancer cells can be followed by comparing the number of cancer cells with decreased SSeCKS expression throughout the treatment. Agents exhibiting efficacy are those which are capable of decreasing the number of cancer cells with decreased SSeCKS.

### 5.4.2. Gene Therapy Applications

The present invention provides compositions and methods that may be used to treat proliferative disorders wherein nucleic acid molecules encoding SSeCKS are administered to modulate cell proliferation. Various delivery systems are known and can be used to transfer the compositions of the invention into cells, e.g. encapsulation in liposomes, microparticles, microcapsules, recombinant cells capable of expressing the composition, receptor-mediated endocytosis (see, e.g., Wu and Wu, 1987, J. Biol. Chem. 262:4429-4432), construction of a nucleic acid as part of a retroviral or other vector, injection of DNA, electroporation, calcium phosphate mediated transfection, etc.

The compositions and methods can be used to treat proliferative disorders such as cancer. In a preferred embodiment, nucleic acids comprising a sequence encoding SSeCKS, or variants thereof, are administered to modulate cell proliferation, by way of gene delivery and expression into a host cell. In this embodiment of the invention, the nucleic acid mediates an effect by promoting SSeCKS production. Variants of SSeCKS that may be expressed include SSeCKS mutants, peptide fragments, and/or fusion proteins. In a specific embodiment of the invention recombinant nucleic acid molecules may be engineered to express variants of SSeCKS that, for example, are capable of sequestering cyclin D in the cytoplasm thereby preventing translocation into the nucleus and induction of cell proliferation. Such variants may include fusion proteins comprising the SSeCKS cyclin D binding domain and a peptide domain capable of anchoring the fusion protein to cytoskeletal components such as actin. Alternatively, mutants of SSeCKS may be expressed within a cell to modulate cell proliferation. Such mutants, include but are not limited to, alterations at the two major PKC sites, SER⁵⁰⁷ and/or SER⁵¹⁵.

Any of the methods for gene delivery into a host cell available in the art can be used according to the present invention. For general reviews of the methods of gene delivery see Strauss, M. and Barranger, J. A., 1997, Concepts in Gene Therapy, by Walter de Gruyter & Co., Berlin; Goldspiel et al., 1993, Clinical Pharmacy 12:488-505; Wu and Wu, 1991, Biotherapy 3:87-95; Tolstoshev, 1993, Ann. Rev. Pharmacol. Toxicol. 33:573-596; Mulligan, 1993, Science 260:926-932; and Morgan and Anderson, 1993, Ann. Rev. Biochem. 62:191-217; 1993, TIBTECH 11(5):155-215. Exemplary methods are described below.

Delivery of the nucleic acid into a host cell may be either direct, in which case the host is directly exposed to the nucleic acid or nucleic acid-carrying vector, or indirect, in which case, host cells are first transformed with the nucleic acid in vitro, then transplanted into the host.

In a specific embodiment, the nucleic acid is directly administered in vivo, where it is expressed to produce the SSeCKS. This can be accomplished by any of numerous methods known in the art, e.g., by constructing it as part of an appropriate nucleic acid expression vector and administering it so that it becomes intracellular, e.g. by infection using a defective or attenuated retroviral or other viral vector (see U.S. Pat. No. 4,980,286), or by direct injection of naked DNA, or by use of microparticle bombardment (e.g., a gene gun; Biolistic, Dupont), or coating with lipids or cell-surface receptors or transfecting agents, encapsulation in liposomes, microparticles, or microcapsules, or by administering it in linkage to a peptide which is known to enter the nucleus, by administering it in linkage to a ligand subject to receptor-mediated endocytosis (see e.g., Wu and Wu, 1987, J. Biol. Chem. 262:4429-4432).

In a specific embodiment, a viral vector that contains a SSeCKS encoding nucleic acid molecule can be used. For example, a retroviral vector can be utilized that has been modified to delete retroviral sequences that are not necessary for packaging of the viral genome and integration into host cell DNA (see Miller et al., 1993, Meth. Enzymol. 217:581-599). Alternatively, adenoviral or adeno-associated viral vectors can be used for gene delivery to cells or tissues. (See, Kozarsky and Wilson, 1993, Current Opinion in Genetics and Development 3:499-503 for a review of adenovirus-based gene delivery).

Another approach to gene delivery into a cell involves transferring a gene to cells in tissue culture by such methods as electroporation, lipofection, calcium phosphate mediated transfection, or viral infection. Usually, the method of transfer includes the transfer of a selectable marker to the cells. The cells are then placed under selection to isolate those cells that have taken up and are expressing the transferred gene. The resulting recombinant cells can be delivered to a host by various methods known in the art. In a preferred embodiment, the cell used for gene delivery is autologous to the host cell.

The present invention also provides for pharmaceutical compositions comprising an effective amount of a nucleic acid encoding SSeCKS, and a pharmaceutically acceptable carrier. In a specific embodiment, the term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, adjuvant, excipient, or vehicle with which the therapeutic is administered. Examples of suitable pharmaceutical carriers are described in “Remington's Pharmaceutical sciences” by E. W. Martin.

In specific embodiments, pharmaceutical compositions are administered: (1) in diseases or disorders involving an absence or decreased (relative to normal or desired) level of SSeCKS protein or function, for example, in hosts where the protein is lacking, genetically defective, biologically inactive or underactive, or under expressed.

The compositions will be administered in amounts which are effective to produce the desired effect in the targeted cell. Effective dosages of the compositions can be determined through procedures well known to those in the art which address such parameters as biological half-life, bioavailability and toxicity. The amount of the composition of the invention which will be effective will depend on the nature of the disease or disorder being treated, and can be determined by standard clinical techniques. In addition, in vitro assays may optionally be employed to help identify optimal dosage ranges.

### 5.4.3. Screening Assays for Agents Useful in Modulating the Activity of SSeCKS Mediated Cyclin D Activity

The present invention relates to screening assay systems designed to identify agents or compositions that modulate SSeCKS activity, and thus, may be useful for modulation of cell proliferation. In accordance with the invention, assay systems can be used to identify agents that regulate the activity of SSeCKS.

The present invention provides methods for identifying an agent that activates SSeCKS activity comprising (i) contacting a cell expressing SSeCKS with a test agent and measuring the level of SSeCKS activity; (ii) in a separate experiment, contacting a cell expressing SSeCKS protein with a vehicle control and measuring the level of SSeCKS activity where the conditions are essentially the same as in part (i), and then (iii) comparing the level of SSeCKS activity measured in part (i) with the level of SSeCKS activity in part (ii), wherein an increased level of SSeCKS activity in the presence of the test agent compared to the level of SSeCKS activity in the presence of vehicle control indicates that the test agent is a SSeCKS activator.

The present invention also provides methods for identifying an agent that inhibits SSeCKS activity comprising (i) contacting a cell expressing SSeCKS with a test agent in the presence of a mitogenic stimulator and measuring the level of SSeCKS activity; (ii) in a separate experiment, contacting a cell expressing SSeCKS in the presence of a mitogenic stimulator and measuring the level of SSeCKS activity, where the conditions are essentially the same as in part (i); and then (iii) comparing the level of SSeCKS activity measured in part (i) with the level of SSeCKS activity in part (ii), wherein a decrease in the level of SSeCKS activity in the presence of the test agent compared to the level of SSeCKS activity in the presence of vehicle control indicates that the test agent is a SSeCKS inhibitor.

In utilizing such systems, the cells expressing the SSeCKS protein are exposed to a test agent or to a vehicle control (e.g., placebo). After or during exposure, the cells can be assayed to measure the activity of SSeCKS or the activity of the SSeCKS dependent signal transduction pathway itself.

The ability of a test molecule to modulate the activity of SSeCKS can be measured using standard biochemical and physiological techniques, e.g., as measured by a chemical, physiological, biological or phenotypic change, induction of a host cell gene or reporter gene, change in host cell kinase activity, etc. For example, the expression of genes known to be modulated by activation of the SSeCKS signal transduction pathway, such as cyclin D, can be assayed to identify modulators of SSeCKS or activity.

In accordance with the invention, an assay system can be used to screen for agents that modulate the expression of SSeCKS within a cell. Assays can be designed to screen for agents that regulate SSeCKS expression at the transcriptional level. The assays described below are designed for identification of agents capable of regulating SSeCKS gene expression.

In one embodiment, DNA encoding a reporter molecule can be linked to a regulatory element of the SSeCKS gene and used in appropriate intact cells, cell extracts or lysates to identify agents that modulate SSeCKS gene expression. Such reporter molecules include but are not limited to chloramphenicol acetyltransferase (CAT), luciferase, B-glucuronidase (GUS), growth hormone, or placental alkaline phosphatase. Such constructs are introduced into cells, thereby providing a recombinant cell useful for screening assays designed to identify modulators of SSeCKS gene expression.

In a specific embodiment of the invention, DNA encoding a reporter molecule can be linked to a regulatory element of the SSeCKS gene and introduced into ras or src, for example, transformed cells where the transcription of SSeCKS is normally repressed. Following exposure of the cells to a test agent the level of reporter gene activity can be quantitated to identify agents capable of de-repressing SSeCKS expression. Such agents would be potentially useful agents for inhibiting cell proliferation.

Following exposure of the cells to the test agent, the level of reporter gene expression can be quantitated to determine the test agent's ability to regulate SSeCKS expression. Alkaline phosphatase assays are particularly useful in the practice of the invention where the enzyme is secreted from the cell, and tissue culture supernatant can then be assayed for secreted alkaline phosphatase. In addition, alkaline phosphatase activity can be measured by calorimetric, bioluminescent or chemiluminescent assays such as those described in Bronstein, I. et al., 1994, Biotechniques 17:172-177. Such assays provide a simple, sensitive, easily automatable detection system for pharmaceutical screening.

In accordance with the invention, assays can be developed to identify agents that modulate transcriptional repression mediated by SSeCKS protein. While not being bound to any one particular theory, it is believed that the SSeCKS protein inhibits the transcription of cyclin D thereby modulating cell proliferation. Thus, in a specific embodiment of the invention, constructs containing a cyclin D responsive element can be linked to any of a variety of different reporter genes and introduced into cells expressing SSeCKS (See, Triesman, 1994, Curr Opin Genet Dev. 4:96-101). Such reporter genes, as set forth above, can include but are not limited to those encoding chloramphenicol acetyltransferase (CAT), luciferase, GUS, growth hormone, or placental alkaline phosphatase. In instances where identification of agonists of SSeCKS repressed transcription is desired the cells are exposed to a mitogenic stimulator and test agent, and the level of reporter gene expression is quantitated to determine the test agent's ability to regulate transcription of the reporter. Alkaline phosphatase assays are particularly useful in the practice of the invention because the enzyme is secreted from the cell. Therefore, tissue culture supernatant can be assayed for secreted alkaline phosphatase. In addition, alkaline phosphatase activity can be measured by calorimetric, bioluminescent or chemilumenscent assays such as those described above.

In addition, as described herein, SSeCKS is capable of inhibiting the translocation of cyclin D into the nucleus thereby inhibiting cell proliferation. In accordance with the invention, an assay can be used to identify agents that modulate translocation of cyclin D protein into the nucleus. For purposes of the assay the cyclin D protein can be tagged with an easily detectable peptide tag such as GFP. Such an assay involves contacting a cell expressing a tagged cyclin D protein with a test agent in the presence of a mitogenic stimulator. Following exposure to the test agent, the amount of tagged cyclin D located within the nucleus is measured, e.g., by measuring the amount of tagged protein present in the nucleus. If the amount of tagged protein detected in the nucleus is decreased in the presence of the test agent, as compared to the same assay conducted in the presence of a vehicle control, a modulator of cyclin D nuclear translocation has been identified.

In a specific embodiment of the invention, SSeCKS fusion polypeptides may be designed that are capable of anchoring cyclin D to the cytoplasmic region of the cell thereby preventing translocation of cyclin D into the nucleus and induction of cell proliferation. Such fusion polypeptides comprise a SSeCKS cyclin D binding domain and a protein domain capable of anchoring the fusion protein within the cytoplasm. As indicated herein, the SSeCKS cyclin binding domain maps to a region designated CY. The SSeCKS gene encodes two closely spaced CY domains, KKLFSXXXXKKLSG [(K/R)(K/R) followed by two non-polar residues. Anchoring domains include those domains that target binding to, for example, structural membrane proteins, cytoskeletal components or cellular organelles located with in the cytoplasm (See, Lester et al., 1997, Recent. Prog. Horm. Res. 52:409-29; Diviani et al., 2001, J. Cell. Sci. 114:1431-7).

### 5.4. Agents that can be Screened in Accordance with the Invention

The assays described above can identify agents that modulate SSeCKS activity. For example, agents that affect SSeCKS activity include but are not limited to agents that bind to SSeCKS and modulate the activity of SSeCKS. Alternatively, agents can be identified that do not bind directly to SSeCKS but are capable of altering SSeCKS activity by altering the activity of a protein involved in SSeCKS signal transduction. Further, agents that affect SSeCKS gene activity (by affecting SSeCKS gene expression, including molecules, e.g., proteins or small organic molecules, that affect can be modulated) can be identified using the screens of the invention.

The agents which may be screened in accordance with the invention can include, but are not limited to, small organic or inorganic agents, peptides, antibodies and fragments thereof, and other organic agents (e.g., peptidomimetics) that bind to SSeCKS and either mimic the activity triggered by activation of SSeCKS (i.e., agonists) or inhibit the activity of SSeCKS (i.e., antagonists).

Agents can include, but are not limited to, peptides such as, for example, soluble peptides, such as members of random peptide libraries (see, e.g., Lam, K. S. et al., 1991, Nature 354:82-84; Houghten, R. et al., 1991, Nature 354:84-86); and combinatorial chemistry-derived molecular libraries made of D- and/or L-configuration amino acids, phosphopeptides (such as members of random or partially degenerate, directed phosphopeptide libraries); (see, e.g., Songyang, Z. et al., 1993, Cell 72:767-778), antibodies (such as polyclonal, monoclonal, humanized, anti-idiotypic, chimeric or single chain antibodies, and FAb, F(ab′)2 and FAb expression library fragments, and epitope binding fragments thereof), and small organic or inorganic molecules.

Other agents that can be screened in accordance with the invention include but are not limited to small organic molecules that affect the expression of the SSeCKS gene or some other gene involved in the SSeCKS signal transduction pathway (e.g., by interacting with the regulatory region or transcription factors involved in gene expression); or such agents that affect the activities of the SSeCKS or the activity of some other factor involved in modulating SSeCKS activity, such as for example, a protein that modifies SSeCKS and thereby activates SSeCKS activities.

### 6. EXAMPLE

### Cloning and Characterization of SSeCKS

cDNAs were identified whose abundance is low in NIH 3T3 cells and decreased following the expression of the activated oncogene v-src. The transcription of one such gene, SSeCKS (pronounced “ESSEX”), was found to be suppressed at least 15-fold in src, ras, and fos-transformed cells and 3-fold in myc-transformed cells, but was unaffected in raf, mos, or neu-transformed cells. Activation of a ts-v-src temperature sensitive allele in confluent 3Y1 fibroblasts resulted in an initial increase in SSeCKS mRNA levels after 1 to 2 hours followed by a rapid decrease to suppressed levels after 4 to 8 hours. Morphological transformation was not detected until 12 hours later, indicating that the accumulation of SSeCKS transcripts is regulated by v-src and not as a consequence of transformation. Addition of fetal calf serum to starved sub-confluent NIH 3T3 or 3Y1 fibroblasts resulted in a similar biphasic regulation of SSeCKS, indicating that SSeCKS transcription is responsive to mitogenic factors. Sequence analysis of a SSeCKS cDNA rat clone (5.4 kb) identified a large open reading frame encoding a 148.1 kDa product, but in vitro transcription-translation from a T7 promoter resulted in a 207 kDa product. Further, sequence analysis indicated that SSeCKS has only limited homology to known genes, including the human gravin gene, where a small amount of homology exists in the 3′ untranslated region. Particular data relating to these conclusions is set forth in greater detail below.

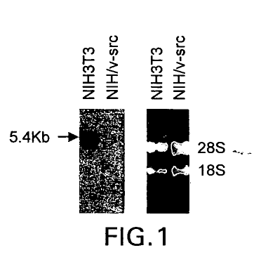

FIG. 1 depicts the results of Northern blot analysis of SSeCKS RNA levels in NIH 3T3 cells versus NIH/v-src transformed cells. A 30 microgram amount of total RNA purified by the RNAzol method from NIH 3T3 cells or NIH/v-src cells was electrophoresed through a 1% agarose-formaldehyde gel, blotted onto Immulon N membrane, hybridized with a 32P-labeled cDNA insert containing SSeCKS sequence, washed, and autoradiographed for 3 weeks. The amount of RNA loaded was normalized by densitometric analysis of 28S and 18S RNA bands (right panel).

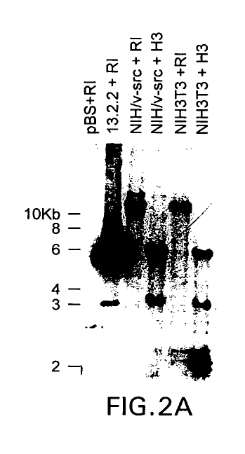

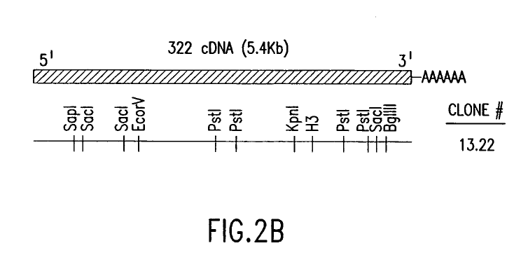

FIG. 2 shows that the decreased level of SSeCKS RNA in NIH/v-src cells is not due to gross deletion or translocation of the SSeCKS allele. As shown in the top panel, a 20 microgram amount of genomic DNA from NIH 3T3 or NIV v-src cells was digested to completion with EcoRI or HindIII, electrophoresed through a 0.7% agarose gel, and then blotted onto Immobilon N membrane. Fifty picogram amounts of EcoRI-cut pBluescript II KS and SSeCKS plasmid DNA were included as negative and positive controls, respectively. The blot was hybridized as described in the legend to FIG. 1, and autoradiographed for 2 days with an intensifying screen. DNA molecular size standards are shown on the left. RI refers to EcoRI, H3 refers to HindIII. The bottom panel shows the restriction map of fall length SSeCKS RNA, and clone 13.2.2, isolated from a rat 3T3 library. Much of this restriction pattern is shared by both mouse and rat SSeCKS homologs, although only the rat allele contains an internal EcoRI site approximately 250 bp from the 3′ cDNA terminus.

FIG. 3 depicts the nucleic acid (top line, lower case letters; (SEQ. ID NO:1)) and deduced amino acid (lower line, capital letters; (SEQ ID NO:2)) sequence of rat SSeCKS cDNA. The largest open reading frame (from bases 176 to 4213) was identified using the TRANSLATE program from Genetics Computer Group (by J, Devereux, 1993, in Madison, Wis.). Glycine-rich domains in the N-terminus are underlined. Nuclear localization signals fitting th motif K(R/K)X(R/K) are boxed. A sequence consistent with a Zn finger from bases 3211 to 3280 is in boldface type. Two polyadenylation signals (AATAAA) in the 3′ untranslated region are underlined.

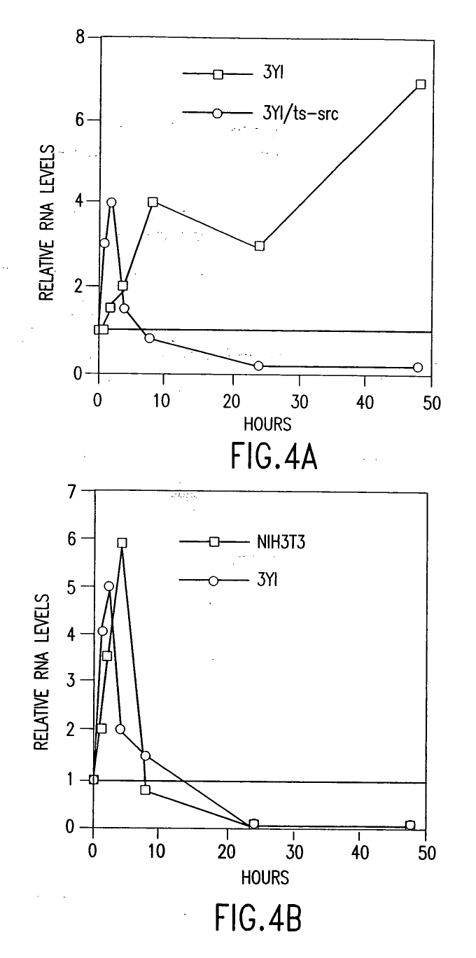

FIG. 4 shows that the transcription of SSeCKS was suppressed relatively soon after the activation of a ts-src allele or the addition of fetal calf serum (FCS) to starved rodent fibroblasts. FIG. 4A depicts the results of experiments wherein 3Y1/ts72src cells or parental rat 3Y1 fibroblasts were grown at the nonpermissive temperature (NPT; 39.5 degrees C.) for 24 hours and then shifted to the permissive temperature (PT) for v-src activity (35 degrees C.). Morphological transformation was not apparent until roughly 24 hours after the temperature downshift. The level of SSeCKS RNA dropped precipitously in the transformed cells but not their untransformed counterparts. FIG. 4B shows the results of experiments in which NIH 3T3 cells and 3Y1 cells were incubated overnight with 0.25% FCS and then with 10% FCS. Total RNA isolated at various times from each cell line was analyzed for SSeCKS transcription by Northern blot analysis using ³²P-labelled SSeCKS probe. Soon after the addition of 10% FCS, the levels of SSeCKS decreased rapidly in both cell lines. The cells used for panel A were seeded at confluency at the start of the experiment whereas the cells used for panel B were subconfluent throughout the experiment.

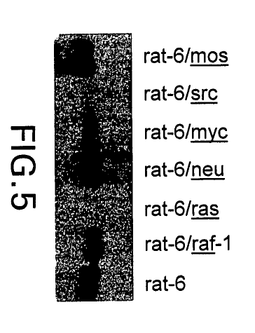

FIG. 5 shows the results of Northern blot analyses showing levels of SSeCKS transcripts in oncogene-transformed Rat-6 fibroblasts, and demonstrates that the transcription of SSeCKS was suppressed at least 15-fold in cells transformed by src and ras and roughly 3 to 4-fold in myc-transformed cells. Each lane of the gel used to generate the blot contained 30 micrograms of total RNA from Rat-6 cells transformed with the oncogenes indicated. The rat-6 lane contains total RNA from normal control cells. The levels of SSeCKS were also found to be down-regulated 10-fold in fos-transformed cells.

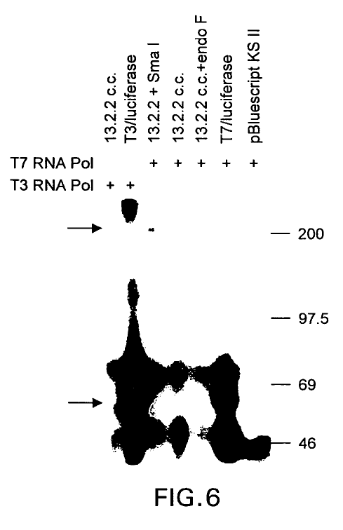

FIG. 6 shows the results of in vitro transcription/translation of SSeCKS cDNA. The SSeCKS cDNA was cloned in a pBluescript II KS vector downstream of the T7 promoter, and analyzed by a coupled in vitro transcription/translation assay (TNT kit, Promega). In contrast to what was predicted, namely, a product with a molecular mass of 148.1 kDa, the 13.2.2 insert repeatedly yielded a 207 kDA product, as shown in the figure.

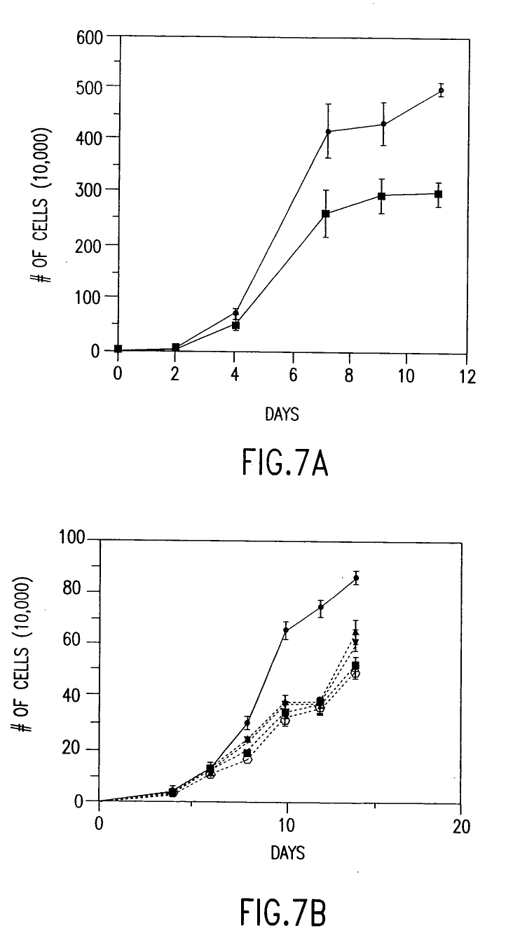

FIG. 7 shows the results of experiments which tested the effect of SSeCKS expression on the proliferation rates of untransformed packaging cells (NIH 3T3 background; panel A) or transformed cells (NIH/v-src; panel B) in the presence of serum growth factors. The SSeCKS cDNA (clone 13.2.2) was inserted into vector pBABEhygro, and transfected stably into the packaging cells (panel A, solid circles). Vector alone was also transfected into these cells (open circles). Proliferation of the cells containing SSeCKS cDNA or vector alone was measured and compared (FIG. 7A). The cells were grown in media supplemented with 10% CS. FIG. 7A shows that after 4 weeks of passage, the growth rate of cells containing SSeCKS cDNA was 40% lower than that of cells containing vector alone.

Filtered supernatants from these packaging cell lines were used to infect NIH 3T3, Rat-6 and NIH/v-src -cells. Although the numbers of hygromycin resistant Rat-6 colonies arising from infection with the vector were similar to those arising from infection with SSeCKS, the initial growth rates of the colonies differed significantly. After 2 weeks, Rat-6/vector colonies were 3 to 5 mm in diameter whereas the Rat-6/SSeCKS colonies contained only 20-50 cells, indicating that SSeCKS is a negative regulator of mitogenesis.

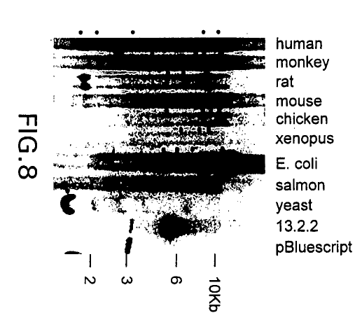

FIG. 8 depicts the results of a Southern “Zoo” blot which measured hybridization of SSeCKS probe to DNA from a variety of species, namely genomic DNA from human (derived from HeLA cells), monkey (from CV-1 cells), rat (from Rat-6 cells), mouse (from NIH 3T3 cells), chicken (from chick embryo fibroblasts), Xenopus (from oocytes), E. coli (strain DH10), salmon sperm, and yeast cells. FIG. 8 confirms that rat and mouse 322 sequences are highly homologous. Furthermore, SSeCKS showed partial cross-hybridization to EcoRI bands from human, monkey, chicken, Xenopus, yeast, and E. coli DNA.

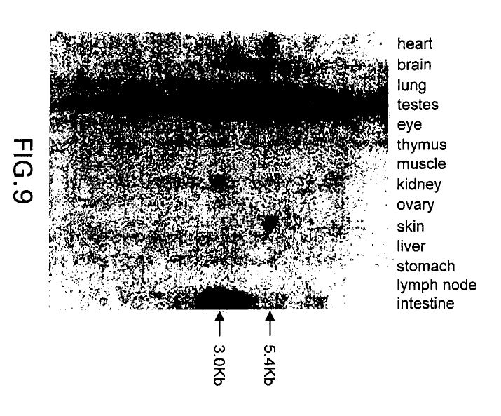

FIG. 9 depicts the results of Northern blot analysis of SSeCKS expression in various mouse tissues. Approximately 6 kb transcripts were found to be abundantly expressed in testes, with 5-10 fold lower levels in skin, brain, and lung. A 3 kb transcript was also detected in intestines, with lower levels in kidney and stomach.

### 7. EXAMPLE

### SSeCKS is a Src- and Ras-Suppressed Protein Kinase C Substrate Associated with Cytoskeletal Architecture

### 7.1. Materials and Methods

Plasmids: A full-length SSeCKS cDNA was con-structed by splicing a 1.2 kB XhoI/BstEII fragment from a 5′RACE clone, p53ext2 (FIG. 11), into a BSTEII/XhoI fragment of clone 13.2.2 SSeCKS cDNA (Lin et al., 1995, Mol. Cell. Biol. 15:2754-2762). The resulting full-length SSeCKS cDNA, the nucleic acid sequence of which is set forth in FIG. 11 (SEQ ID NO:3), was sequenced using Sequenase 2.0 kits and the data submitted to Genebank in updated form. GST fusion constructs were produced using pGEX-5x-1 (Pharmacia) and His-tag constructs were produced using pET28 (Novagen). Retroviral constructs of the full-length SSeCKS cDNA were produced in pBABE hygro and packaged in cells as described in (Gelman and Hanafusa, 1993, Oncogene 8:2995-3004). The SSeCKS cDNA was also spliced into pCEV27 (Miki et al., 1991, Proc. Nat'l. Acad. Sci. 88:5167-5171) containing the Moloney leukemia virus (MLV) LTR promoter, and stably transfected into Rat-6 cells (Borner et al., 1992, J. Biol. Chem. 267:12900-12910) followed by selection on 400 mg/ml of genetic (Life Sciences) as described in (Gelman and Hanafusa, 1993, Oncogene 8:2995-3004).

Expression of GST- and His-tag fusion proteins: A fragment of the SSeCKS open reading frame (amino acid residues 389 to 894; FIG. 11; SEQ ID NO:4) was amplified by PCR from the 13.2.2 cDNA using primers 322-13 (cDNA coordinates 1167 to 1184; FIG. 11; SEQ ID NO:3) and 322-11b (cDNA coordinates 2725 to 2710; FIG. 11; SEQ ID NO:3), and cloned in frame into EcoRI/XhoI-cut pGEX-5x-1 or pET28a, resulting in clones GST-322 and His-322, respectively. Another fusion product, GST-1322, was produced by PCR amplifying a 4 kb fragment from 13.2.2 cDNA using primers 322-13 and 322-36 (5637 to 5623; FIG. 11; SEQ ID NO:3), cutting the fragment with EcoRI and BglII, cloning into pBluescript SK II (Stratagene), excising the EcoRI/SalI fragment and splicing into EcoRI SalI-cut pGEX-5x-1. The GST fusion clones containing the “SSeCKS 1-4” protein kinase C (“PKC”) phosphorylation sites were PCR amplified using the following primers, and then cloned in frame into pGEX-5x-1: SSeCKS-1 (amino acid residues 275-390; FIG. 11; SEQ ID NO:4), primers 322-PS5 (cDNA coordinates 825-842; FIG. 11; SEQ ID NO:3) and M13 reverse primer for p53ext DNA template, and cutting with EcoRI/NotI; SSeCKS-2 (amino acid residues 389-552; FIG. 11; SEQ IS NO:3), produced by an internal SacI deletion of the GST-322 construct; SSeCKS-3, primers 322-PS6 (cDNA coordinates 1758-1775; FIG. 11; SEQ ID NO:3) and 322-24 (cDNA coordinates 2010-2180; FIG. 11; SEQ ID NO:3). Additional clones include: SSeCKS-¼, primers 322-PS5 (above) and 322-11 (above); SSeCKS- 2/4. which is the same as GST-322; and SSeCKS ¾, primers 322-PS6 (above) and 322-11 (above). The resulting clones were sequenced with Sequenase 2.0 kits and clones which lacked Taq polymerase errors were picked. BL21 (DE3)pLysS bacteria (Novagen) transformed with these plasmids were grown in LB media to OD=6 at 37° C., and then grown for an additional hour at 30° C. in LB plus 0.1 mM IPTG to induce protein expression. The pelleted bacteria were resuspended in 3 ml per gram of bacterial pellet in buffer A (50 mM Tris-HCl, pH 8.0, 1 mM EDTA, 100 mM NaCl, 1 mM NaF, 0.5 mM sodium vanadate, 2 mg/ml each of trasylol, leupeptin, antipain, pepstatin A and 1 mM PMSF) and lysed by 3-5 cycles of freeze-thawing. Debris was removed by centrifugation for 10 minutes at 3 K (4° C.). This was followed by the sequential addition of 4 mg deoxycholic acid per gram of bacterial pellet (while stirring, until viscous), MgSO4 to a final concentration of 5 mM, and then 300 U benzonase (Boehringer Mannheim). After 30 minutes incubation on ice, the lysate was checked for loss of viscosity using a pasteur pipette. Debris was then pelleted by cen-trifugation at 16 K for 15 minutes at 4° C. The supernatant was applied to either a glutathione-Sepharose column (Pharmacia) or a Ni2+-Sepharose column (Qiagen). The column beds were washed according to the manufacturer's specification, and the bound protein was eluted with 3 washes of either glutathione (15 mM) or imidazole (250 mM) for the GST or His-tag proteins, respectively. The purity of the proteins was determined by electrophoresis on 6% SDS-poly-acrylamide stacking mini-gels followed by Coomassie blue staining, and it the case of GST proteins, by Western blotting (Gelman and Hanafusa, 1993, Oncogene 8:2995-3004) onto Immobilon-P (Millipore) and probing with rabbit anti-GST sera. Protein concentrations were determined using BioRad assay reagent (Bradford).

Production of immune sera: After approximately 10 ml of pre-immune sera was obtained, two New Zealand giant rabbits were immunized with 150 mg each of GST-1322 protein emulsified with an equal volume of complete Freund's adjuvant (Life Technologies). The rabbits were boosted 2-3 times more with 50-100 mng/injection of GST-1322 in incomplete Freund's adjuvant. The specificity of the sera was determined by probing slot blots containing GST protein alone, GST-1322, His6-1322, and BL21 lysate alone, followed by incubation with alkaline phosphatase-labeled sheep anti-rabbit Ig (Boehringer-Mannheim), washing in Western blot buffer (below), and developing with BCIP/NCP (Promega). Both rabbits gave high titers (>5000) of anti-SSeCKS antibodies. Immunoaffinity-purified anti-SSeCKS antibodies were isolated as follows: Glutathione-Sepharse columns were saturated with either GST or GST-1322, washed and then treated with 25.5 mM dimethyl pimelimidate cross-linker (Pierce). 10 ml of RB anti-SSeCKS sera was passed repeatedly over the GST column (the bound antibodies were eluted with glutathione after each round) until all the anti-GST reactivity (as determined by slot-blot Western analysis) was removed. The resulting sera was passed over the GST-1322 column, and the bound antibodies were eluted with Tris-glycine buffer, pH 2.8, as described in (Sambrook et al., 1989, Molecular Cloning: A Laboratory Manual, 2 nd Ed., Cold Spring Harbor Laboratory Press, Cold Spring Harbor, N.Y.). This fraction was shown by slot blot Western analysis to retain GST-1332 and His6-1322 binding at dilutions of 1:1000, and no cross-reactivity to GST protein alone at dilutions of 1:100.

In vitro transcription/translation of SSeCKS: Plasmid DNAs (1 mg) containing either the full-length SSeCKS cDNA or the 5.4 kb 13.2.2 cDNA cloned into Bluescript SKII were linearized at the 3′ ends of the cDNA inserts (SMAI) and incubated at 30° C. for 90 minutes in a 50 ml coupled transcription/translation reaction (TNT; Promega) containing 50 mCi of translation-grade [³⁵S] methionine (New England Nuclear), according to the manufacturer's specification. 5 ml of the resulting protein products were electrophoresed on a 6% SDS-polyacrylamide stacking gel (above). The gels were fixed in methanol/acetic acid (15%/7%, respectively), incubated in Amplify (Amersham) and fluorographed with Kodak XAR film at 70° C.

In vitro PKC phosphorylation assay: PKC assays were variations of assays described in (Kobayashi et al., 189, BBRC 159:548-553). Briefly, 40 ml reactions contained 10 ml of 0.3 mg/ml of the target polypeptide, 10 ml of 1 mCi/,251 [³²P]ATP (New England Nuclear), 10 ml rabbit brain PKC enzyme (10-25 ng), and 10 ml of 4× buffer (20 mM Tris-HCl, pH 7.5, 0.1 mM CaCl², 5 mM MgCl²; 0.03% Triton X-100, and freshly added 0.31 mg/ml L-phosphatidyl-L-serine [PS], 0.06 mg/ml 1,2-dileoyl-rac-glycerol, and 0.4 mM ATP) were incubated for 30 minutes at 37° C. Target proteins included various GST-SSeCKS products, PKC substrate peptide[Ser25]PKC, and the PKC substrate peptide ac-Myelin Basic Protein [4-14] (the latter two from Life Technologies). A PKC-specific inhibitor (pseudosubstrate) peptide PKC[19-36] (life Technologies) was used at 0.15 mM. 10 ml of phosphorylated product was analyzed using SDS-PAGE as described above.

In vivo phosphorylation analysis: 10⁶ Rat-6 cells were incubated overnight in DEM (Bio-Whittaker) supplemented with 0.5% calf serum (Life Technologies) and then twice for 1 h in DEM without sodium phosphate (Life Technologies). Labeling was for 2 hours in MEM without phosphate supplemented with 0.5% calf serum (Life Technologies) and then twice for 1 h in DEM without sodium phosphate (Life Technologies). Labeling was for 2 hours in MEM without phos-phate supplemented with 150 mCi of [³²P]orthophosphate (New England Nuclear). In some case, phorbol 12-myristate 13-acetate (PMA; 200 nM) was added for various durations at the end of this labeling period. The PKC-specific inhibitor, bis-indolylmaleimide (Boehringer Mannheim; 10 mM), was added at the beginning of the labeling period and again when PMA was added. After washing the cells thrice with ice-cold PBS, the cells were lysed in 0.5 ml RIPA/150 mM NaCl and analyzed by SKS-PAGE as described above.

Western (immuno-) blot analysis: Cells were washed thrice in ice-cold phosphate buffered saline (PBS), lysed in 1 ml/10 cm plate with RIPA buffer containing 150 mM NaCl (Gelman et al., 1993, Oncogene 8:2995-3004), vortexed, incubated on ice for 10 min, and then centrifuged at 13K for 30 min at 4° to remove debris. 50-400 mg of cell lysate was electrophoresed through 6% SDS-polyacrylamide stacking gels, and then electrophoretically transferred to Immobilon-P. A rapid immunodetection method was followed (M. A. Mansfield, Millipore Corp.) in which dried blots were not re-wetted, and then processed as described previously (Gelman, et al., 1993, Oncogene 8:2995-3004) using PBS containing 1% non-fat dry milk (Difco) and 0.05% Tween-20 (Sigma) as the buffer. Alkaline phosphatase-labeled secondary antibodies were either sheep anti-rabbit Ig or sheep anti-mouse Ig (Boehringer Mannheim), and the substrate was room-temperature stabilized BCIP/NCP solution. Protein concentrations were determined using a Micro BCA Protein Assay Kit (Pierce).

Co-precipitation (pull-down) assay: 1 mg of lysate from Rat-6 or Rat-6/PKC overexpressor cells (gifts of I. B. Weinstein, Columbia University) (Borner et al., 1995, J. Biol. Chem. 270:78-86), or 20 ng of purified rabbit brain PKC (Upstate Biologicals, Inc.) were co-incubated with 135 ml of glutathione-Sepharose pre-bound to 50 mg of GST-1322 for 4 h at 4° C. (rotating) in RIPA buffer containing 150 mM NaCl, 5 mM MgCl². PS was added in some cases at 0.37 mg/ml. The pellets were washed thrice and then analyzed by SDS-PAGE and immunoblotting as described above using mouse monoclonal (MAb) anti-PKC type III (Upstate Biologicals, Inc.)

Subcellular fractionation of plasma membrane and cytosol components: 10⁶ Rat-6 or Rat-6/PKC over-expressor cells were washed thrice in ice-cold Tris-Glu buffer (25 mM Tris-HCl, pH 7.4, 150 mM NaCl, 5 mM KCl, 1 mM sodium phosphate, 0.1% glucose). The cells were scraped into Tris-Glu, and pelleted by centrifugation at 1.5K for 5 min. The cells were swollen on ice for 10 min in 20 mM Tris-HCl, pH 7.4., 10 mM KCl, 1 mM EDTA, 1 mM DTT, 1% trasylol and 1 mM PMSF. The cells were dounce homogenized (40 strokes with pestle B), and then NaCl was added to a final concentration of 100 mM. The nuclei and cell debris were pelleted at 1.5K for 10 min. (4° C.) yielding initial pellet (P1) and supernatant (S1) fractions.

The S1 fraction was loaded into polycarbonate tubes and centrifuged in a SW41 rotor (Beckman) for 30 min. at 100,000 g, yielding a secondary pellet (P100), containing plasma membranes, and supernatant (S100). Aliquots of these fractions were analyzed by SDS-PAGE and immunoblotting as above.

Immunofluorescence analysis: Rat-6 cells were seeded onto sterile 22 mm² coverslips at a density of roughly 70% and then incubated overnight or until the cells were confluent for at least 2 days. The coverslips were washed thrice in ice-cold PBS and the cells were fixed in 60% acetone/3.7% formaldehyde for 20 min at −20° C. as described previously (Gelman and Silverstein, 1986, J. Mol. Biol. 191:395-409). After washing in PBS, the cells were incubated for 1 h with immunoaffinity-purified rabbit polyclonal anti-SSeCKS (above; 1:50 dilution) and rhodamine-labeled phalloidin (1:400; Sigma). Secondary antibodies to detect SSeCKS were fluorescein-labeled anti-rabbit Ig(Boehringer Mannheim). The coverslips were mounted in Aqua-Mount (Lerner Laboratories, Pittsburgh, Pa.) containing 20 mM p-phenylenediamine (Kodak) as an anti-bleaching agent.

Reagents: All regents were purchased from Sigma unless indicated otherwise.

### 7.2. Results

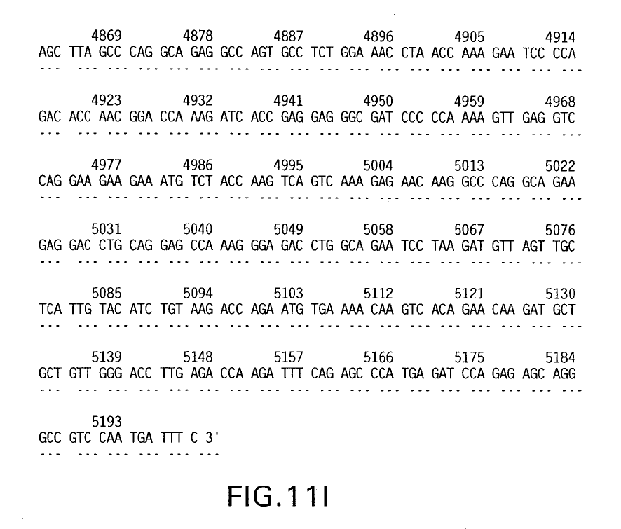

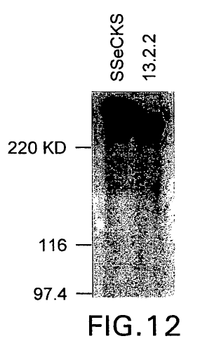

SSeCKS is identical to the “>200 kDa” PKC substrate. Several novel substrates of PKC have been identified using overlay assays (Hyatt et al., 1994, Cell Growth and Differ-entiation 5:495-502). The SSeCKS protein appears to be identical to the so-called “>200 kDa” protein identified in that study for the following reasons: (i) recent attempts by the authors of the study to clone the >200 kDa protein yielded partial cDNAs with greater than 99 percent sequence homology to the SSeCKS sequence (in Genbank as the 322 sequence, U23146); and (ii) SSeCKS and the >200 kDa protein share many idiosyncratic characteristics such as resistance to heat denaturation, in vitro phosphorylation by PKC, and phosphatidylserine-dependent binding to PKC (Hyatt et al., 1994, Cell Growth and Differentiation 5:495-502). A 5′-RACE product, p53ext2 was spliced to the 5.4 Kb cDNA described in the foregoing section (FIG. 3) in order to construct a full-length SSeCKS cDNA having a length of 6.0 kb (FIG. 11). FIG. 12 shows that both the upstream and internal ATG sites are independently recognized in T7 transcription/translation system (TNT; Progmega), although the internal site is silence in the context of the upstream sire. Most importantly, the product obtained in vitro from the upstream ATG has a similar mobility in SDS-PAGE to native SSeCKS (280/290 kDA form).

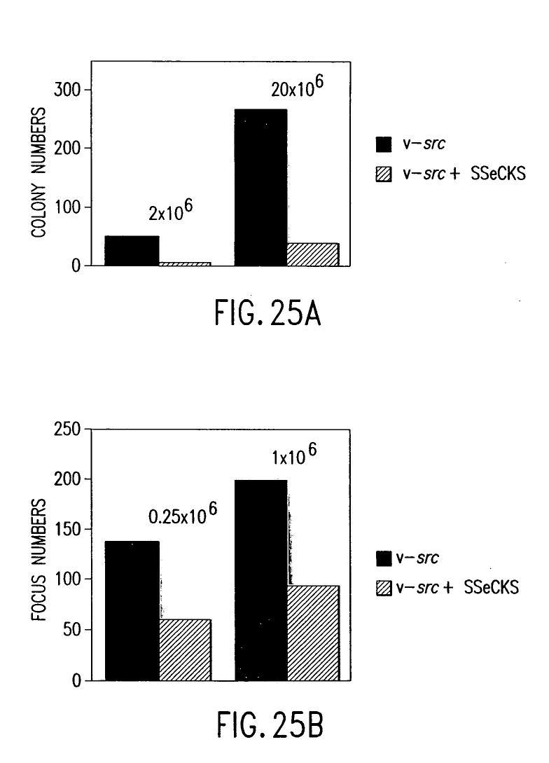

As discussed above, constitutive expression of the SSeCKS truncated protein (encoding amino acids 387-1594; FIG. 11; SEQ ID NO:4) led to the selection of cells containing deletions of the transduced SSeCKS cDNA. In this section, we evaluated whether over expression of the full length SSeCKS cDNA, via a retroviral vector (pBABEhygro) or a vector driven by the MLV-LTR promoter (pCEV27), was toxic in rodent fibroblasts. We found that the presence of the full-length SSeCKS cDNA severely decreased, by more than 95 percent, the frequency of hygromycin resistant colony formation following either infection with packaged virus or direct DNA transformation of both untransformed and src-transformed cells. The few colonies that did arise showed highly unstable growth characteristics, such as a more than 80 percent decrease in cell viability following trypsinization. These data indicate that high level constitutive expression of full-length SSeCKS or an N-terminal truncated SSeCKS version (see Section 6) is growth inhibitory. FIG. 25 shows that the transient transfection of full-length SSeCKS decreases v-src-induced colony formation in soft agar and focus formation. pMv-src (1 mg) and SSeCKS-pBabehygro (10 mg) or pBabehygro (10 mg) plasmids were co-transfected into NIH3T3 cells. After two days of transfection, 2×10⁶ and 20×10⁶ cells were used in a soft agar colony formation assay, the results of which are depicted in FIG. 25A. 0.25×10⁶ and 1×10⁶ cells were cultured for the formation of foci (also initiated two days after transfection); the results are shown in FIG. 25B. The suppression of morphological transformation may be due to SSeCK's ability to induce grown arrest or to a selective ability to suppress parameters of transformation in the absence of growth arrest.

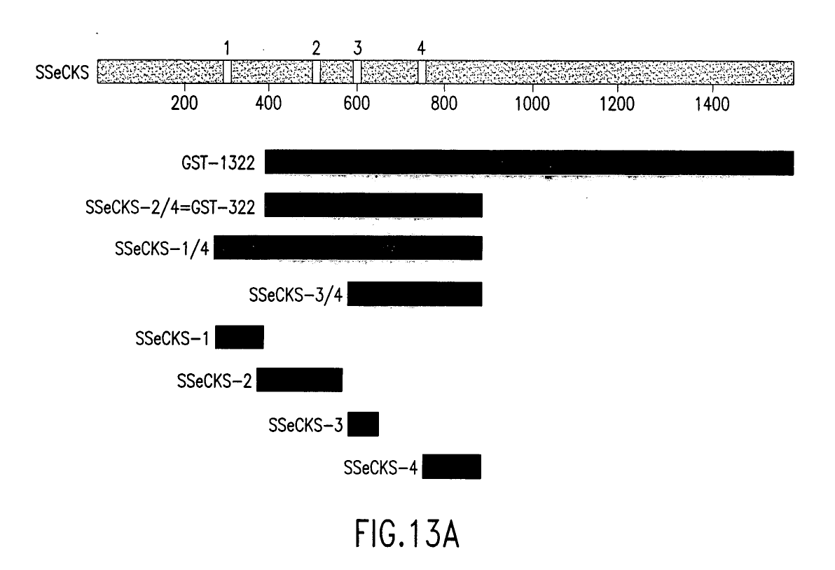

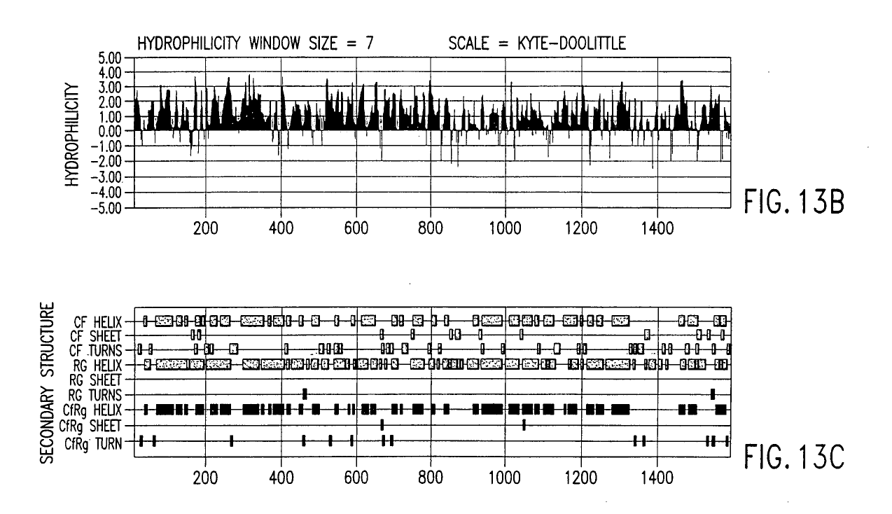

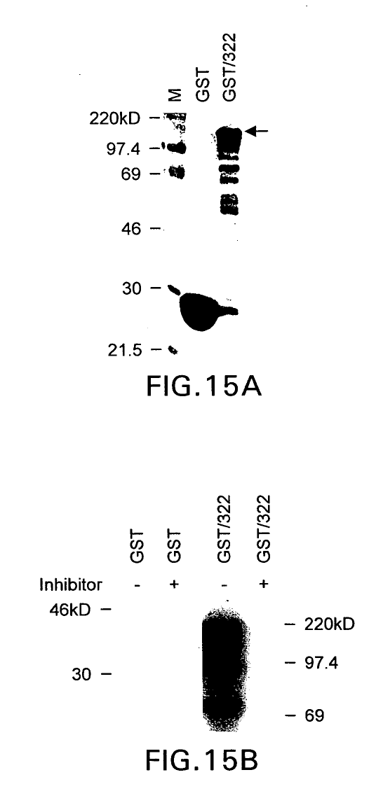

Expression of the 322 ORF products and production of specific antisera. The predicted structure of SSeCKS using the Chou-Fassman algorithm (Chou and Fasman, 1978, Advances in Enzymiology 47:45-147) was that of an elongated, rod-shaped protein with a concentration of both CF turns and predicted antigenic sites (Jameson-Wolf index; Wolf et al., 1987, Computer applications in the Biosciences 4:187-191) roughly one third into the coding sequence (FIG. 13). FIG. 15 shows the inducible expression and purification of a GST-322 fusion protein with an apparent mobility on SDS-PAGE of 160 kDa even though the predicted molecular weight is 81 kDa (including GST). This result is consistent with the description in the preceding section of a retarded mobility for the 322 ORF product (the truncated protein), and most likely results from a high concentration of acidic residues as well as an inherent rod-like structure. The smaller polypeptides purified on glutathione columns are considered C-terminal breakdown products inasmuch as Western blotting using anti-GST sera identified the same band pattern as Coomassie blue staining.

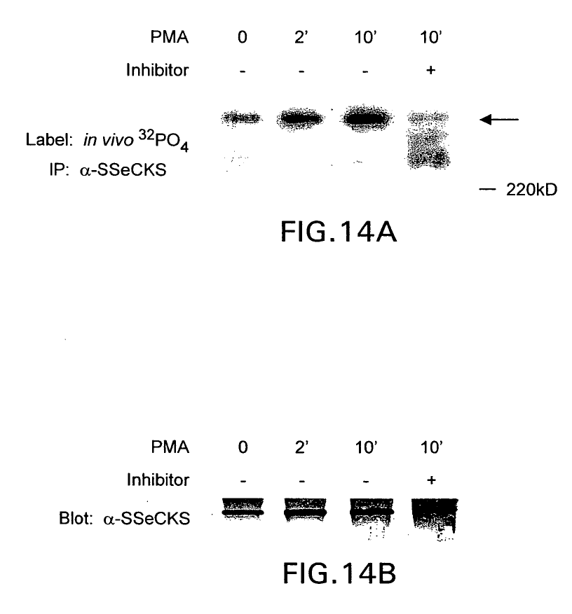

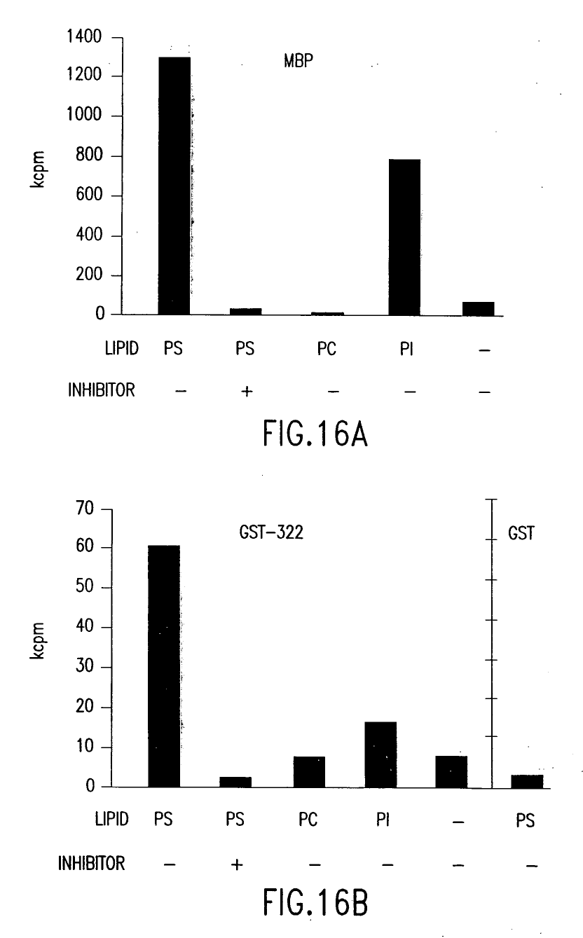

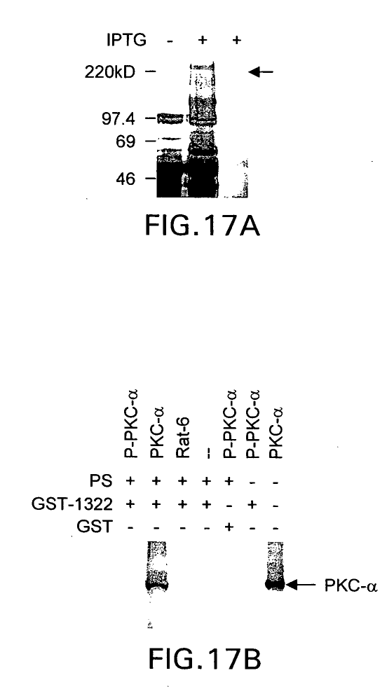

In order to characterize the forms of SSeCKS expressed in Rat-6 fibroblasts, rabbit antisera was raised against the purified GST-322 protein. FIGS. 14, 20 and 21 show that three SSeCKS protein species, a 240 kDa and a 280/290 protein doublet, are identified by Western blot analysis using immune rabbit serum (in FIG. 14, the 280/290 doublet is unresolved). Other minor protein species were identified in immunoblots using anti-SSeCKS sera: 120, 95, 60 and 44 KDa. These may represent proteolytically cleaved forms of the larger SSeCKS species (240, 280, 290 KDa) or products of spliced SSeCKS RNAs. The 280/290 kDa doublet form is consistent with the in vitro product generated using the TNT system (FIG. 12) and the bacterially expressed GST-SSeCKS products (FIGS. 15 and 17), further suggesting that posttranslational modifications are only minor contributors to the molecular weights of the mature SSeCKS products in Rat-6 cells. The 240 kDa species may represent either an in vivo utilization of the internal ATG site or a specific proteolytic cleavage product. Interestingly, the 240 kDa species in Rat-6 cells is not readily labeled in vivo with [³²P]orthophosphate (FIG. 14, top panel), although predicted casein kinase II and PKC sites are found throughout the SSeCKS ORF. In view of data disclosed in the preceding section which showed a single mRNA hybridizing to SSeCKS cDNA probe, the multiple forms of SSeCKS protein observed are most likely due to protein modifications rather than to multiple SSeCKS allelic products.

It was somewhat difficult to metabolically label SSeCKS protein in either subconfluent or confluent cultures using either [³⁵S]-methionine/cysteine or [³H]-leucine, although p60c-src was easily labeled in the same lysates. This could not be due to a dearth of Met, Cys or Leu residues in SSeCKS (which occur 20, 15, and 86 times, respectively, in rat SSeCKS). In contrast, SSeCKS could be immunoblotted easily under the same conditions, suggesting that its relative rate of de novo synthesis is low. SSeCKS is not glycosylated in an in vitro mammalian translation system. The addition of tunicamycin to Rat-6 cells did not alter the electrophoretic mobility of SSeCKS as determined by [³⁵S]-methionine/cysteine labeling or Western blotting, indicating that SSeCKS is not significantly glycosylated in vivo.

SSeCKS as a PKC substrate Activation of PKC by shortterm addition of nM concentrations of phorbol esters is known to result in the rapid phosphorylation of PKC substrates such as MARCKS (A. Aderem, 1992, Cell 71:713-716). FIG. 14 (top) indicates that the relative phosphorylation level of the 280/290 kDa species in vivo rapidly increases 5-6 fold in response to PMA, and that this induction is abrogated by the addition of the PKC-specific bis-indolylmaleimide inhibitor, GF-109203X. The PMA-induced phosphorylation effect is apparent in as little as 2 minutes and lasts at least 10 minutes (FIG. 14), but wanes with treatments of longer than 60 minutes although this may reflect down regulation of PKC (Mahoney et al., 1994, in Protein Kinase C (Kuo, J. F. ed) pp. 16-63, Oxford University Press, New York). This rapid PKC-induced phosphorylation is similar to that of MARCKS (A. Aderem, 1992, Cell 71:713-716; Allen and Aderem, 1995, EMBO J. 14:1109-1121), which is often used as a gauge of PKC activation. Although SSeCKS from quiescent Rat-6 fibroblasts contains no detectable phosphotyrosine, as determined by anti-phosphotyrosine immunoblotting, it cannot be ruled out that tyrosine is being phosphorylated following PMA treatment or activation of ts-v-src. However, SSeCKS is not tyrosine phosphorylated in Rat-6/src cells.

It was then determined that purified rat brain PKC containing α, β, and δ isoforms could phosphorylate GST-322 protein in vitro (FIG. 15, panel B). This phosphorylation was inhibited by the addition of excess PKC pseudosubstrate peptide (amino acids 19-36), indicating that PKC and not a contaminating kinase was responsible for phosphorylation. The PKC-specific phosphorylation of GST-322 paralleled that of myelin basic protein (MBP) in its dependence on PS (FIG. 16) and Ca2+. PI could supplant PS in this assay, although the relative level of GST-322 phosphorylation was roughly two-fold less than that of MBP using PI (FIG. 16). As previously reported using MBP, PC did not stimulate PKC activity of GST-322 (Mahoney and Huang, 1994, in Protein Kinase C (Kuo, J. F. ed) pp. 16-63, Oxford University Press, New York). Although several C-terminal breakdown products smaller than 70 kDa were present in the preparation of GST-322 (FIG. 15), only the products that were greater than or equal to 70 kDa were phosphorylated in vitro. This suggests that the PKC sites do not map to the extreme C-terminal portion of SSeCKS in the GST-322 construct. Therefore, these data indicate that SSeCKS is both an in vivo and in vitro substrate of PKC.

SSeCKS binding to PKC. The ability of PKC to phos-phorylate GST-322 indicates some level of interaction between these proteins. Results (FIG. 17) indicate that SSeCKS binds both purified PKC and PKC in Rat-6 lysates in a PS-dependent manner. Thus, SSeCKS and PKC most likely interact via a PS bridge, although it cannot be ruled out that there may be a lower affinity protein-protein inter-action with domains in SSeCKS not encoded by GST-1322. Prephosphorylation of GST-322 by PKC decreases this binding at least 10-fold, suggesting that phosphorylated SSeCKS has decreased binding affinity for PS.

Identification of in vitro PKC phosphorylation sites on SSeCKS. The consensus motifs for PKC phosphorylation have been identified as S/TXK/R or K/RXS/T, with a greater preference for serine over threonine (Pearson and Kemp, 1991, Meth. Enzymol. 200:63-81). However, our observations of previously characterized in vivo PKC phosphorylation sites indicates that they typically contain a high concentration of basic residues and at least 2 or 3 of the overlapping phosphorylation motifs described above. Analysis of the SSeCKS sequence yielded four such putative phosphorylation sites, shown in Table I. These sites share some linear sequence homology and predicted secondary structural similarity with the PKC phosphorylation site in MARCKS. A minimal MARCKS 23-peptide containing this site (Hartwig et al., 1992, Nature 356:618-622) also binds calmodulin and F-actin (Table 1).

TABLE 1

Proposed PKC phosphorylation sites in SSeCKS: Comparison with
known calmodulin-binding and PKC phosphorylation sites.

in the MARCKS protein family and in myosin light chain kinase

| | | | CaM | Actin | PKC |
|---|---|---|---|---|---|
| | Sequence | | binding | binding | phosph. |
| MARCKS (bovine/chicken) | ¹⁵³KRFSSKKSFKLSGFSFKKNKKEA₁₇₇ | (SEQ ID NO:11) | + | + | + |
| MARCKS (mouse) | KRFSSKKSFKLSGFSFKKSKKEA | (SEQ ID NO:12) | + | + | + |
| MacMARCKS/F52 | KKFSSKKPFKLSGFSFR | (SEQ ID NO:13) | + | + | + |
| Myosin light chain kinase^{a} | KRRWKKAFIAVSAAARFKKC | (SEQ ID NO:14) | + | − | ? |
| | WAGWRKK | | | | |
| SSeCKS-1 (rat) | ²⁷⁹ETTSSFKKFFTHGTSFKKSKEDD₃₀₇ | (SEQ ID NO:15) | ? | ? | + |
| SSeCKS-2 (rat) | ⁵⁰⁴KLFSSSGLKKLSGKKQKGKRGGG₅₂₆ | (SEQ ID NO:16) | ? | ? | + |
| SSeCKS-3 (rat) | ⁵⁹²EGITPWASFKKHVTPKKRVRRPS₆₁₄ | (SEQ ID NO:17) | ? | ? | + |
| SSeCKS-4 (rat) | ⁷⁴¹EGVSTWESFKRLVTPRKKSKSKL₇₆₆ | (SEQ ID NO:18) | ? | ? | + |
| 3/4-Consensus | EGV W SFKK VTPKKK K | (SEQ ID NO:19) | | | |
| | I R R R R | (SEQ ID NO:20) | | | |

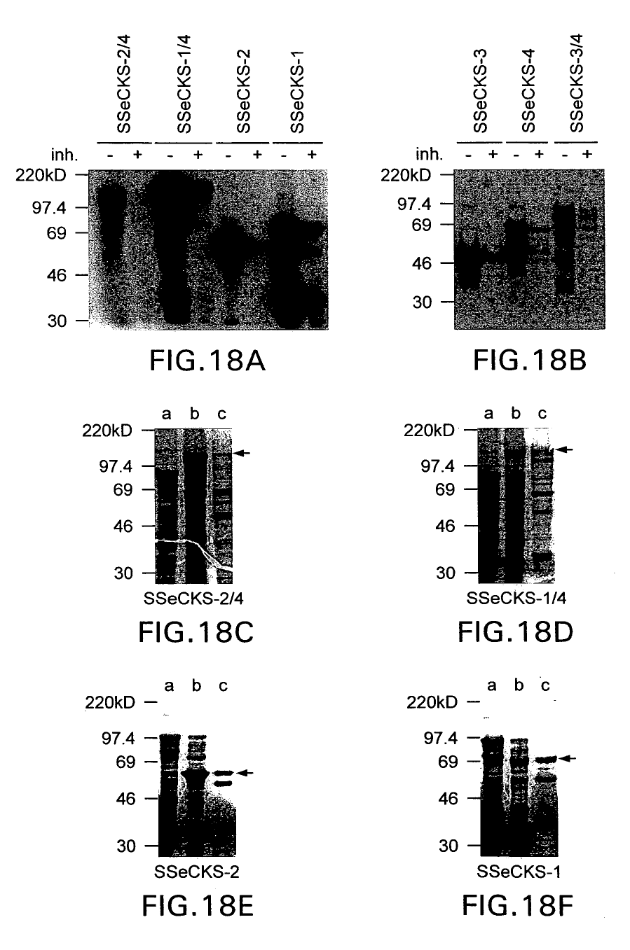

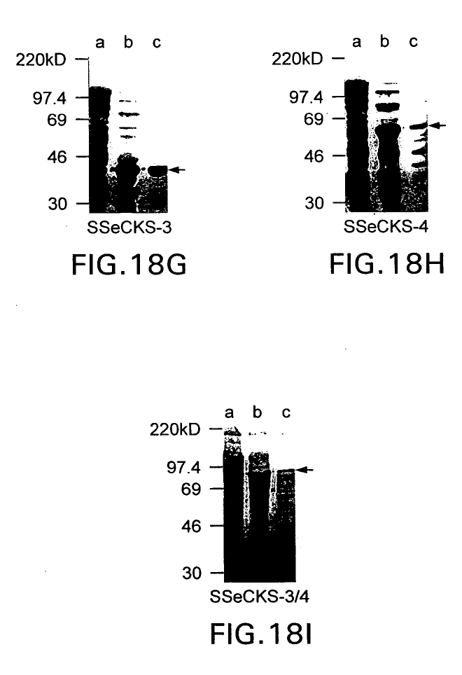

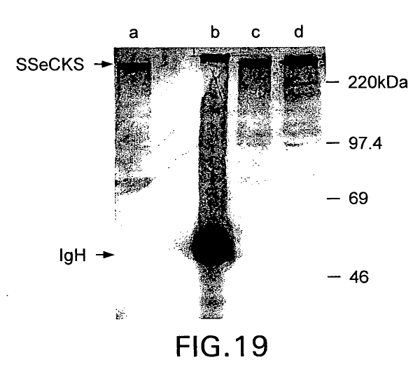

In order to determine whether SSeCKS could be phosphorylated in vitro by PKC, PCR products containing individual predicted PKC phoshorylation sites or several sites (Table 1) in tandem were generated, fused in-frame to GST-expressing vectors (FIG. 13) and checked by sequencing. FIG. 18 indicates that sites 1-4 could be phosphorylated efficiently by purified rabbit brain PKC and that this phosphorylation was blocked by excess pseudo-substrate peptide inhibitor. SseCKS is resistant to heat denaturation Besides having predicted rod-like structures, many PKC substrates share peculiar characteristics such as resistance to heat denaturation (Grohmann et al., 1990, Eur. J. Immunol. 20:629-636; Urbanelli et al., 1989, Virology 173:607-614). FIG. 19 shows that the 280/290 kDa form of SSeCKS remained soluble after 5 minutes of boiling in the absence of SDS. Additionally, boiled SSeCKS retained roughly 50 percent of its immunoreactivity with rabbit immune serum. These data indicate that SSeCKS assumes a rod-like structure in vivo. In contrast, GST fusions of SSeCKS are heat labile PKC substrates, a characteristic most likely conferred by the GST moiety.

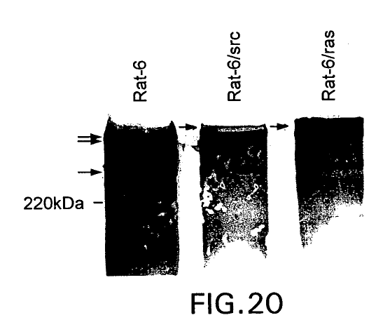

SSeCKS in src and ras transformed cells. The SSeCKS encoding gene (i.e. 322) was originally isolated based on its being transcriptionally suppressed in src-transformed NIH3T3 cells (Frankfort and Gelman, 1995, BBRC 206:916-926). It has also been shown that the gene is down-regulated at least 10-fold at the steady state RNA level in src and ras-transformed Rat-6 fibroblasts but not in cells transformed by activated raf. FIG. 20 shows that the relative level of SSeCKS in src- and ras-transformed Rat-6 fibroblasts is about 10-fold lower than in untransformed cells. An additional 305 kDa protein is found in the Rat-6/ras cells only, which might represent an induced SSeCKS homologue or a modified form of the 280/290 kDa SSeCKS doublet. Thus, the relative abundance of SSeCKS in transformed cells seems to be controlled at the transcriptional level.

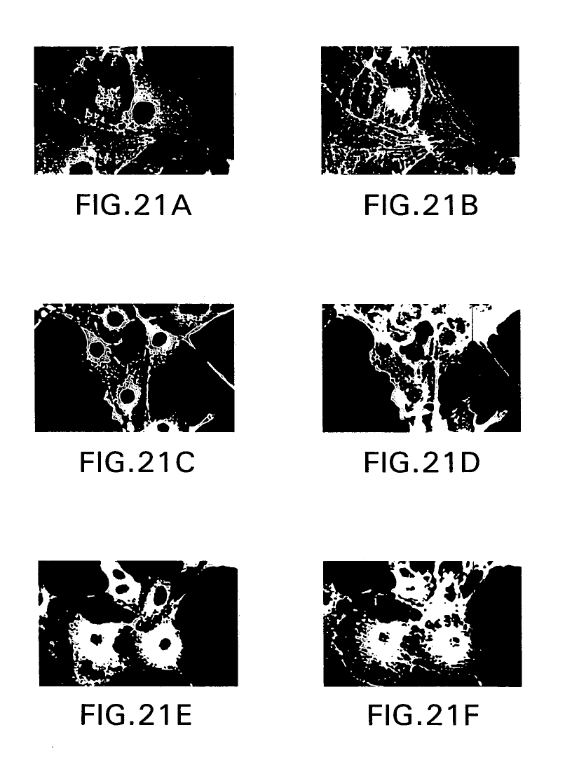

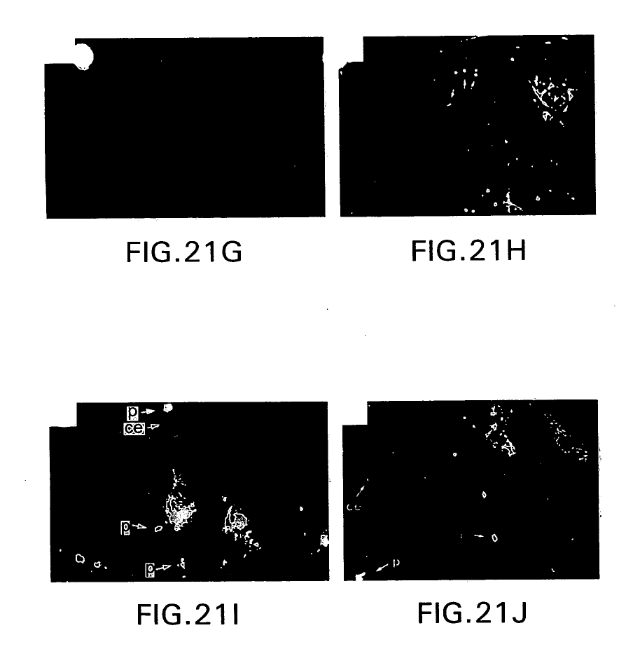

Cell localization of SSeCKS. We determined where SSeCKS is found in subconfluent and confluent Rat-6 cells. Immunofluorescence analysis using immunoaffinity-purified anti-SSeCKS antibody indicates that SSeCKS localizes to the cytoplasm but is enriched at the cell edge, in structures resembling podosomes, and in the perinucleus (FIG. 21). An apparent intranuclear staining of SSeCKS (FIG. 21J) most likely represents deposits of SSeCKS in or on the nuclear cage based on confocal microscopy. The association of SSeCKS with cortical actin like structures (FIGS. 21A, 21C and 21E) and cellular components such as podosomes (FIG. 21I) further supports a role for SSeCKS in the control of actin based cytoskeletal architecture.

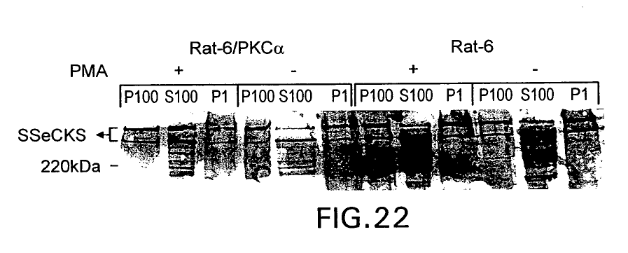

It has been shown that short term treatment of quiescent fibroblasts with PMA led to a rapid detachment of MARCKS from plasma membrane sites into a soluble cytoplasmic compartment, followed by its reassociation with membrane structures and progressive movement towards the perinucleus (Allen and Aderem, 1995, EMBO J. 14:1109-1121). This effect was coincident with a ruffling of actin fibers at the plasma membrane. We determined the effect of PMA on SSeCKS localization. FIG. 21A-F shows that after 10 minutes of PMA treatment, membrane ruffling of actin was apparent in rat fibroblasts, but SSeCKS was still uniformly associated with cortical actin like structures throughout the cytoplasm. With longer PMA treatment (60 minutes), SSeCKS localized predominantly to the perinucleus. This delayed movement of SSeCKS towards the perinucleus, when compared with MARCKS, suggests that it is a consequence of exocytosis, which is known to be induced by short term PMA treatment. We then determined whether PMA treatment causes an initial solubilization of SSeCKS. FIG. 22 shows that the relative level of SSeCKS associated with either membrane or soluble subcellular compartments does not change significantly in Rat-6 cells after 30 minutes of PMA treatment. We cannot rule out that PMA induces the solubilization of a minor, membrane-associated component of SSeCKS. These data suggest that most of SSeCKS, unlike MARCKS, remains tethered to cytoskeletal actin structures during movement towards the perinucleus.

### 7.3. Discussion

The SSeCKS coding sequence contains four domains of overlapping PKC phosphorylation motifs (S/TXK/R or K/RXS/T) representing potential phosphorylation sites. Each of these sites, designated herein as SSeCKS 1-4, can be phosphorylated in vitro by purified rabbit brain PKC in a PS- and calcium ion dependent manner. The I phosphorylation of SSeCKS could also be supported by PI but not by PC, confirming previous data on the phospholipid cofactor requirements of PKC (Mahoney and Huang, 1994, in Protein Kinase C (Kuo, J. F. ed) pp. 16-63, Oxford University Press, New York). Moreover, the binding of SSeCKS to PKCα in vitro is PS-dependent, which is consistent with the PS-dependent binding of PKC by the >200 kDa protein (Hyatt et al., 1994, Cell Growth and differentiation 5:495-502).

The first two PKC phosphorylation sites in SSeCKS (SSeCKS1-2; Table I) contain significant similarities with a 23-mer MARCKS peptide encoding a minimal PKC phosphorylation site as well as binding ability to calmodulin and F-actin (Hartwig eg al., 1992, Nature 356:618-622). These SSeCKS sites also are enriched for basic residues, as has been reported for other PKC sites (Pearson and Kemp, 1991, Meth. Enzymol. 200:63-81). In contrast to SSeCKS-1 and -2 (SEQ ID NOS:15-16), whose sequences are not that similar to each other, SSeCKS-3 and -4 (SEQ ID NOS:17-18) share significant sequence and predicted structural homology, although they are less similar to the MARCKS 23-mer PKC site than SSeCKS-1 or -2. This suggests a coor-dinated or redundant control of the phosphorylation of SSeCKS-3 and -4 in vivo.

The SWISSPROT databank was searched for similarities to the putative serine-phosphorylation sites in SSeCKS 1-4 (Table I), with the requirement that potential phosphoserine residues be retained. No significant similarities to SSeCKS-1 were found. The SSeCKS-2 PKC site showed 50 percent identity to a sequence in the retinoic receptor-β(SWISSPROT:Rra1_Mouse) and the SSeCKS-¾ consensus peptide (SEQ ID NOS:19-20) showed 46.2 percent identity to the A-kinase anchor protein, AKAP-79 (SWISSPROT:Ak79_Human). It is unknown whether these other proteins are phosphorylated by PKC at these sites. However, these similarities to SSeCKS strengthen the notion of a function for SSeCKS at the plasma membrane.

Analysis of the in vitro SSeCKS phosphorylation sites using the HELICAL WHEEL program (J. Devereux, 1993, The GCG Sequence Analysis Software Package, Version 8.0, Genetics Computer Group, Inc. Madison Wis.) predicts amphipathic helical structures for SSeCKS-1, -2, and -4 but less so for SSeCKS-3. It is difficult to predict whether there is any interplay between these phosphorylations sites as they are separated by between 60-100 residues on a proposed rod-shaped molecule. In the case of MARCKS, McLaughlin and Aderem (McLaughlin and Aderem, 1995, TIBS 20:272-276) postulate that MARCKS probably associates with plasma membranes via its N-terminal myristyl group and its concentration of positively charged amino acid residues in the PKC phosphorylation site. PKC phosphorylates three serines in this site that align along one axis of a short amphipathic helix. They further postulate that the resulting confluence of electrostatic phosphoserine charges causes MARCKS to detach from plasma membrane sites. Indeed, SSeCKS is enriched at the cell edge and in podosomes (FIG. 21), as has been demonstrated for MARCKS (A. Aderem, 1992, Cell 71:713-716). However, following the activation of PKC, SSeCKS did not detach appreciably from the membrane sites or from subcellular fractions enriched for plasma membranes (FIG. 21A-F; FIG. 22). This suggests that the phosphoserine charges in SSeCKS are insufficient to counteract its affinity for membranes. It cannot be ruled out that only a minor component of SSeCKS is membrane-associated in the cell and that following PKC induces this component to move into a soluble cytoplasmic compartment.

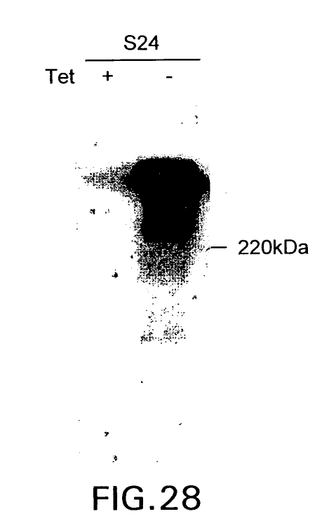

The ability of SSeCKS to associate with plasma membrane sites is predicted by an N-terminal myristylation signal, MGAGSSTEQR, which is similar to signals encoded by retroviral GAGs and the HIV nef product (Anderson and Pastan, 1975, Adv. Cyclic Nucl. Prot. Phosph. Res. 5:681). This sequence lacks the Cys-3 residue shared by members of the src and Gα family which are also palmitylated, with the exception of Gα^{t}/transducin, the signal of which is quite similar to that of SSeCKS and which is myristylated only. Indeed, SSeCKS was demonstrated to be myristylated by in vivo labeling (FIG. 28).

SSeCKS also localizes to the perinucleus and cytoplasm of Rat-6 cells. No intranuclear staining was detected although SSeCKS encodes at least four nuclear localization signals of the adenovirus E1a motif, K/RKXK/R. However, we have detected intranuclear staining the testes, where SSeCKS transcription is highest in the mouse, in a subset of cells in the seminiferous tubules (FIG. 9). SSeCKS may encode both cytoplasmic and nuclear signal and regulatory functions.

Although SSeCKS and MARCKS share little sequence similarity past their PKC phosphorylation sites, they share several biochemical and structural characteristics common to other PKC substrates implicated in the regulation of cytoskeletal architecture such as igloo, GAP-43, and neurogranin. These include (i) a predicted elongated or rod structure; (ii) enrichment for alanine, serine, lysine and glutamic acid residues; (iii) binding to plasma membranes (GAP-43, for example, is palmitoylated); (iv) association with focal contact sites or cellular processes; (v) predicted or proven phospholipid binding activity; and (vi) predicted or proven calmodulin and F-actin binding domains (Mahoney and Huang, 1994, in Protein Kinase C (Kuo, J. F. ed) pp. 16-63, Oxford University Press, New York; Neel and Young, 1994, Development 120:2235-2243; Maekawa et al., 1993, J. Biol. Chem. 268:13703-13709). Additionally, the over-expression of SSeCKS or MARCKS is growth inhibitory. This correlates with the increase in SSeCKS and MARCKS expression as cells enter GO (Lin et al., 1995, Mol. Cell. Biol. 15:2754-2762; Herget et al., 1993, Proc. Nat'l. Acad. Sci. 90:2945-2949). These data suggest that SSeCKS and MARCKS share some overlapping functions and regulatory motifs.

However, unlike MARCKS, which is expressed through-out mammalian tissues, and SSeCKS, which is primarily expressed in the brain, genitourinary tract, intestines and kidney, GAP-43, igloo, and neurogranin are brain-specific (Mahoney and Huang, 1994, in “Protein Kinase” C (Kuo, J. F. ed) pp. 16-63, Oxford University Press, New York; Neel and Young, 1994, Development 120:2235-2243; Maekawa et al., 1993, J. Biol. Chem. 268:13703-13709). Additionally, GAP-43, igloo, and neurogranin, but not MARCKS and SSeCKS, encode PKC phosphorylation sites with the so-called “IQ” motif, KIQASFRGH (Cheney and Mooseker, 1992, Curr. Op. Cell Biol. 4:27-35).

SSeCKS localizes to focal contact sites (FIGS. 21H and I) known to be enriched for PKC, p125FAK, and actin-binding proteins. These structures mediate the interaction of cytoplasmic actin fibers with extracellular matrices via integrins (Luna and Hitt, 1992, Science 258:955-963; Zachary and Rozengurt, 1992, Cell 71:891-894). SSeCKS appears to bind to actin in overlay blots, supporting a role for SSeCKS in the regulation of actin-based cytoskeletal architecture.

Recent data indicate that actin fiber formation is controlled by rac and rho-mediated pathways distinct from the raf/MAP kinase-mediated pathways controlling proliferation (Nobes and Hall, 1995, Cell 81:53-62). SSeCKS transcription is suppressed in src and ras but not raf-transformed cells. Thus, the raf-independent control of SSeCKS expression parallels the rac and rho-dependent control of actin-based cytoskeletal architecture.

### 8. EXAMPLE

### Expression of SSeCKS in Various Human Tissues and Cell Lines

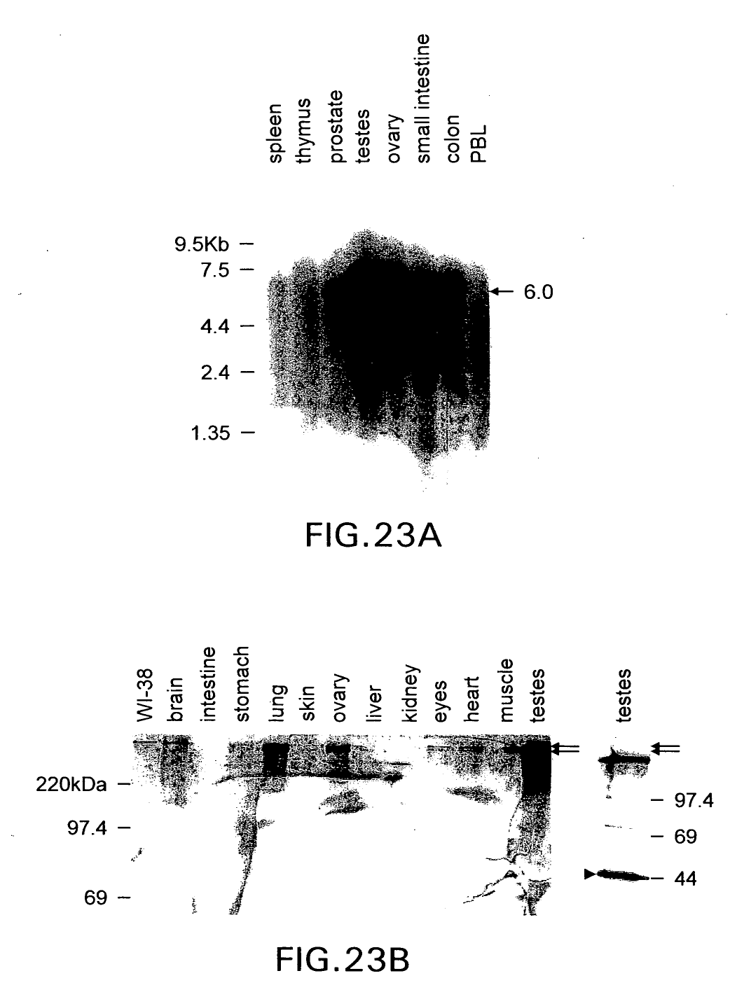

FIG. 23A depicts a Northern blot of RNA prepared from various human tissues, using radiolabeled rat SSeCKS cDNA as a probe. Expression was greatest in human testes, similar to the mouse (see FIG. 9). RNA expression was found to correlate with protein expression; FIG. 23B presents the results of a Western blot analysis of SSeCKS expression in a panel of mouse tissues using antibody directed toward rat SSeCKS protein. Highest levels of SSeCKS protein expression were found in mouse and human testes, with significant amounts of antibody reactive protein in lung and ovary as well. Immunohistochemical analyses indicate a high level of SSeCKS expression in epithelial cells in the human prostate, breast and testes, although expression in fibroblastlike cells was also apparent.

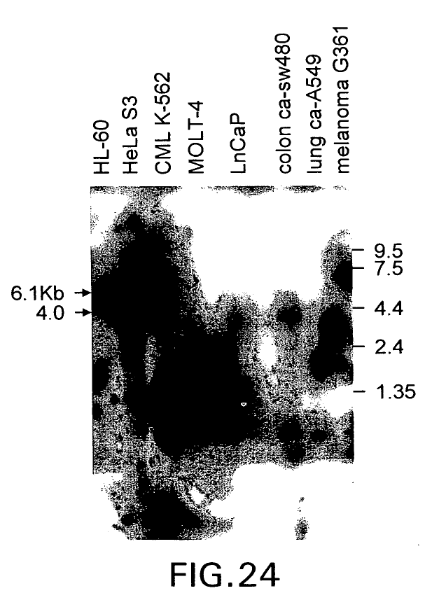

FIG. 24 depicts the results of a Northern blot analysis of RNA collected from a variety of human tumor cell lines. Radiolabeled rat SSeCKS cDNA was used as a probe. Expression was absent or negligible in HL-60, CML K-562, MOLT-4, LnCaP, colon ca-SW480, and melanoma cell line G361. In contrast, two bands, one approximating in size the 6.0 SSeCKS transcript, were detected in HeLa S3 cells. This indicates that SSeCKS may be transcriptionally suppressed in several human cancers, including but not limited to prostate and colon cancer.

### 9. EXAMPLE

### SSeCKS is Myristylated

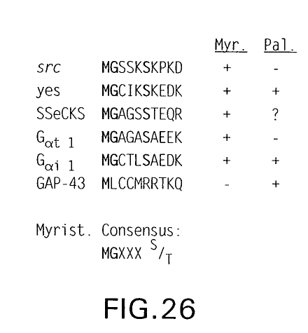

FIG. 26 depicts regions of src, yes, Gαt₁, Gαᵢ, and GAP-43 amino acid sequences (SEQ ID NOS:5, 6, 8, 9 and 10) associated with myristylation and/or palmitylation; the corresponding region found at the N-terminus of rat SSeCKS (SEQ ID NO:7) is compared. Myristylation would facilitate the association of SSeCKS with the plasma membrane.

In order to test whether SSeCKS was myristylated, the full length SSeCKS cDNA was placed under the control of a promoter repressed by the presence of tetracycline, using an expression system based on the tetracycline resistance (tet) operon of E. coli (Gossen and Bujard, 1992, Proc. Natl. Acad. Sci. U.S.A. 89:5547-5551). This system employs a tetracycline-controlled, hybrid transactivator (tTA) that consists of the tet repressor (tetR) and the transcriptional transactivating domain of herpes simplex virus protein 16 (VP16). Tetracycline binds directly to the tetR, inhibiting its DNA binding activity. Removal of tetracycline from the culture medium allows tTA to bind to tet operator sequences placed in front of a minimal mammalian promoter thereby causing rapid induction of cDNAs placed downstream of the tet operator sequences (Gossen and Bujard, 1992, Proc. Natl. Acad. Sci. U.S.A. 89:5547-5551; Schmid, 1995, Trends Cell Biol. 5:266-267).

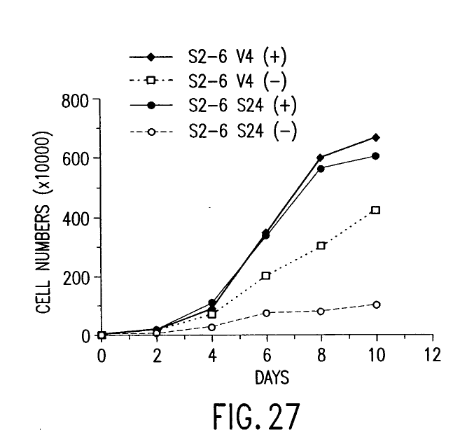

Using this tet repressor system, the full length rat SSeCKS cDNA was inserted into the pUHD15-1. The resulting construct was then transfected into S2-6 cells, Shockett et al., 1995, PNAS, 92:6522-6526, a derivation of NIH3T3. In the presence of tetracycline, SSeCKS was not significantly expressed in the transfectants, but when cells containing the pUHD15-1/SSeCKS construct were placed in tetracycline-free media, SSeCKS expression was induced. FIG. 27 shows that proliferation of transfected cells (clone S2-6/S24) was lower in tetracycline-free medium (−), consistent with the negative effect of SSeCKS on cell proliferation. S2-6/V4 cells, transfected with vector lacking an insert, served as a control.

To evaluate whether SSeCKS is myristylated in vivo, transfected S24 cells containing the SSeCKS/tet-repressor construct were cultured in media containing tritiated myristylate in the presence or absence of tetracycline. The results are shown in FIG. 28, which shows an autoradiograph of SDS-page of a RIPA lysate from tetracycline containing (+) and tetracycline-free (−) cultures. The presence of a myristylated band correlated with the absence of tetracycline and SSeCKS expression, demonstrating that SSeCKS indeed is myristylated in vivo.

### 10. EXAMPLE

### Deficient SSeCKS in Weaver Mice

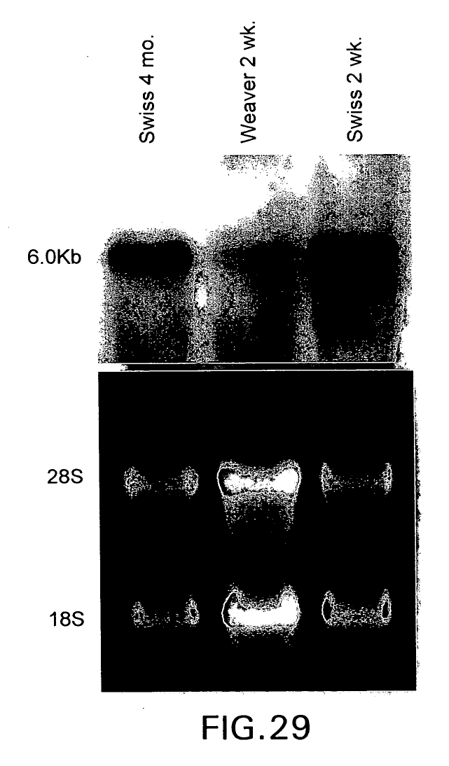

Expression of SSeCKS was evaluated in weaver mice, a mutant mouse strain exhibiting aberrant development of the nervous system and testes (Vogelweid et al., 1993, J. Neuro-genetics 9:89-104). The mutation is believed to involve a receptor associated with the opiate system. As shown in FIG. 29, depicting a Northern blot analysis of SSeCKS RNA levels in normal Swiss and weaver mutant mice, the level of SSeCKS RNA was decreased or absent in weaver mice. Thus, SSeCKS may be involved with the development of the nervous system and testes.

### 11. EXAMPLE

### Relationship of SSeCKS and Cell Morphology

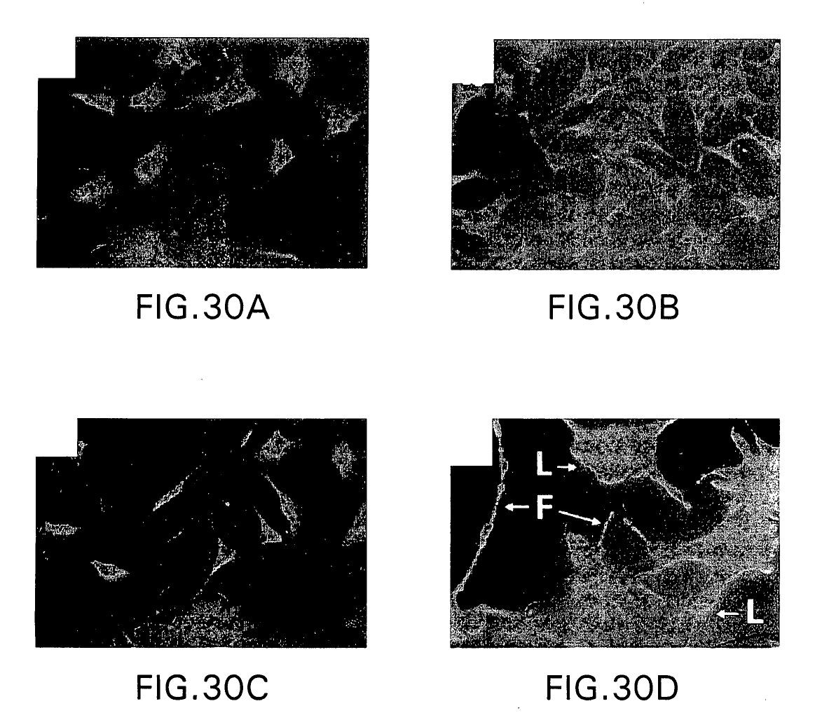

FIG. 30A-D is a series of photomicrographs showing S24 cells transfected with the tetracycline-repressed SSeCKS construct described in section 9, above. FIGS. 30A and B show control cells in media with (A) and without (b) tetracycline (vector alone), exhibiting normal morphology. The morphology of S24 cells in the presence of tetracycline (FIG. 30C) was also normal. In the absence of tetracycline, S24 cells showed increased cell flattening marked by elaboration of the cytoskeletal matrix and increased production of lamellipodia (L) and filopodia (F). The morphological change and growth arrest (FIG. 27) induced by SSeCKS overexpression is suggestive of a terminally differentiated phenotype.

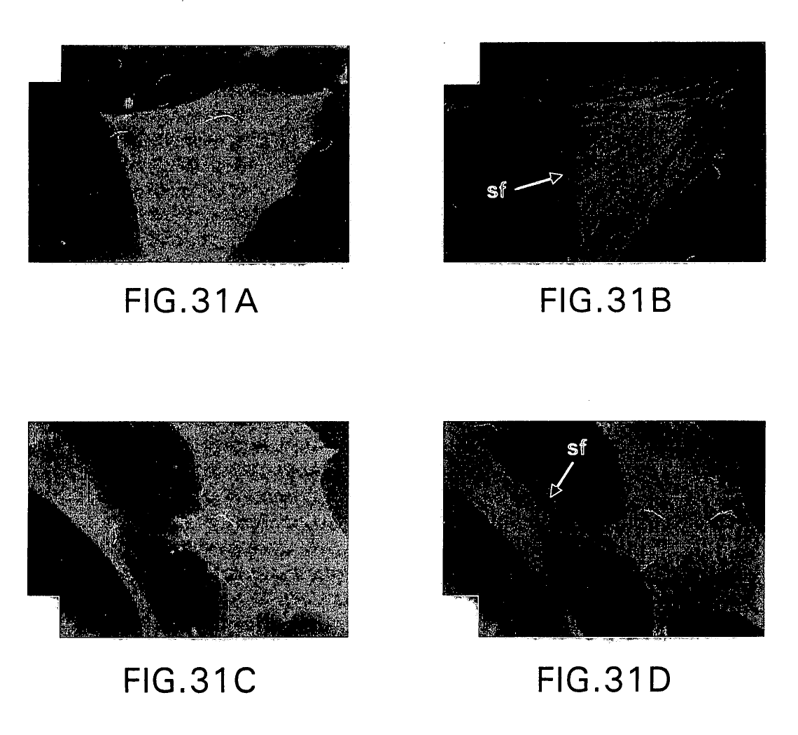

FIG. 31A-D is a series of photomicrographs of the tetracycline-repressed SSeCKS transfected S24 cells stained with fluorescent labeled antibodies to SSeCKS (FIGS. 31A and 31C) and actin (FIGS. 31B and 31D) in the presence (FIGS. 31A and 31B) and absence (FIGS. 31C and 31D) of tetracycline. SSeCKS overexpression (FIGS. 31C and D) was associated with a loss of actin stress fibers (sf). This is consistent with a role for SSeCKS in the early events of cytoskeleton-associated cell motility of cytokinesis.

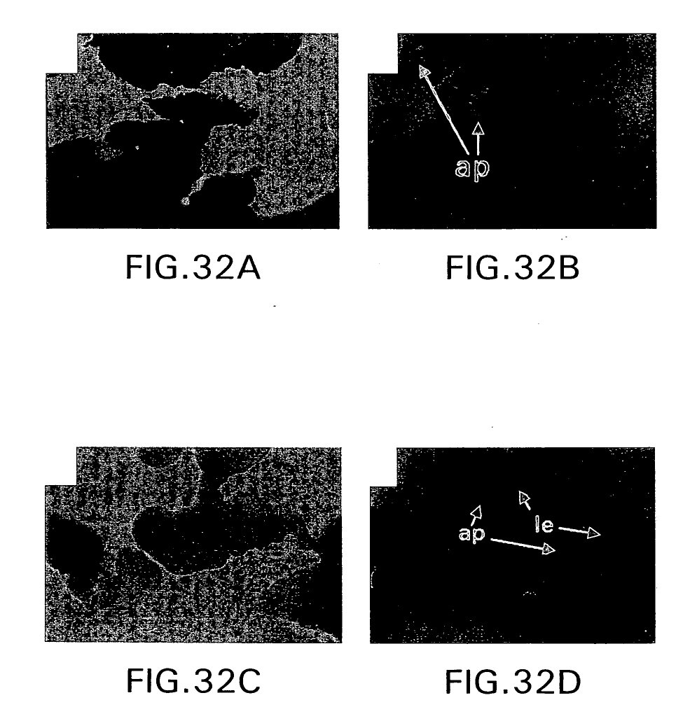

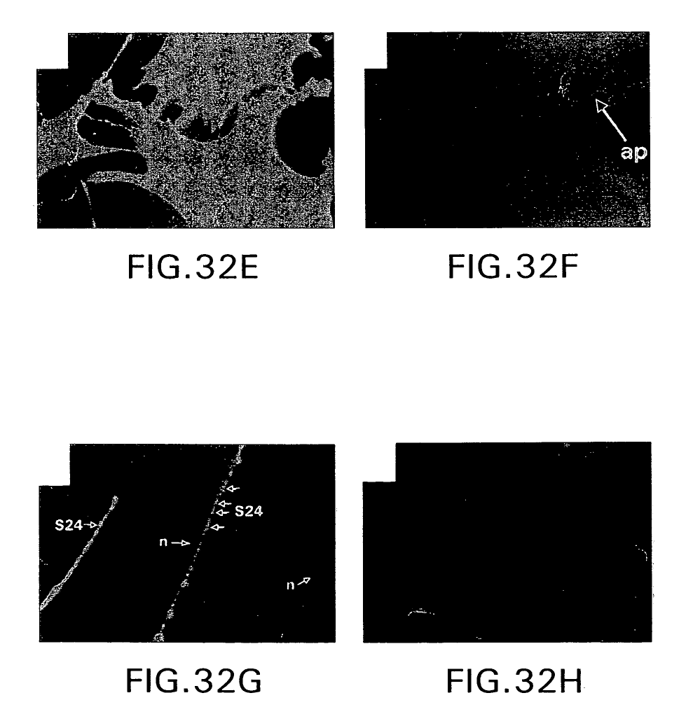

FIG. 32A-H shows a similar series of photomicrographs of S24 transfectants in the absence of tetracycline for 1 (a, b, g, h), 3 (c, d) and 4 (e, f) days, after which the cells were stained for SSeCKS (a, c, e and g) and vinculin (b, d, f, and h). After 1 day, adhesion plaques are detected only in the cell not overexpressing SSeCKS (left cell, panel a/b). After 3 days, adhesion plaques began to form in the SSeCKS overexpressor cells but were not located at the cells' leading edges (le). After 4 days, adhesion plaques were detected at the leading edge in the SSeCKS overexpressor cells. Panels g and h show the inverse expression pattern of SSeCKS and vinculin in filo-podia of overexpressor (S24) and non-overexpressor cells (n). Thus, the initial overexpression of SSeCKS delays the formation of vinculin-associated adhesion plaques. Taken together with the result in FIG. 31, this strongly suggests that SSeCKS controls cytoskeletal matrix formation associated with motility or cytokinesis.

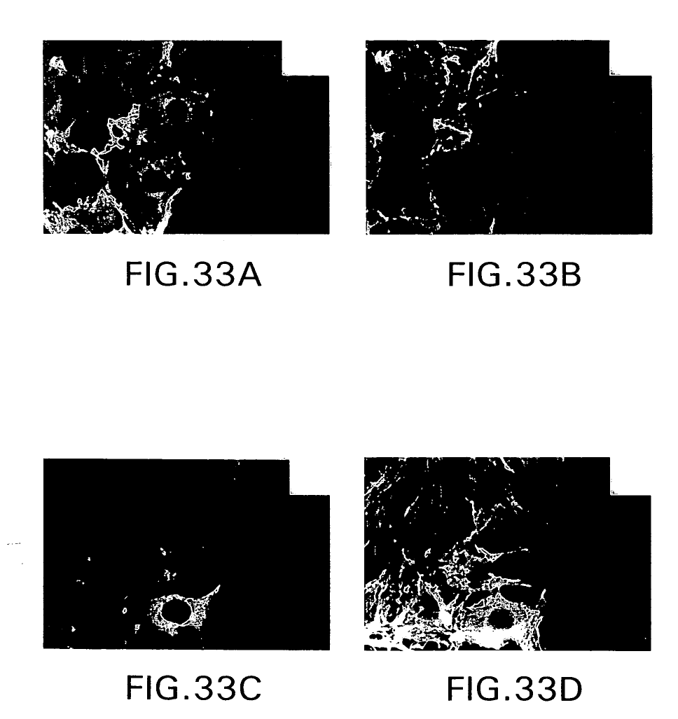

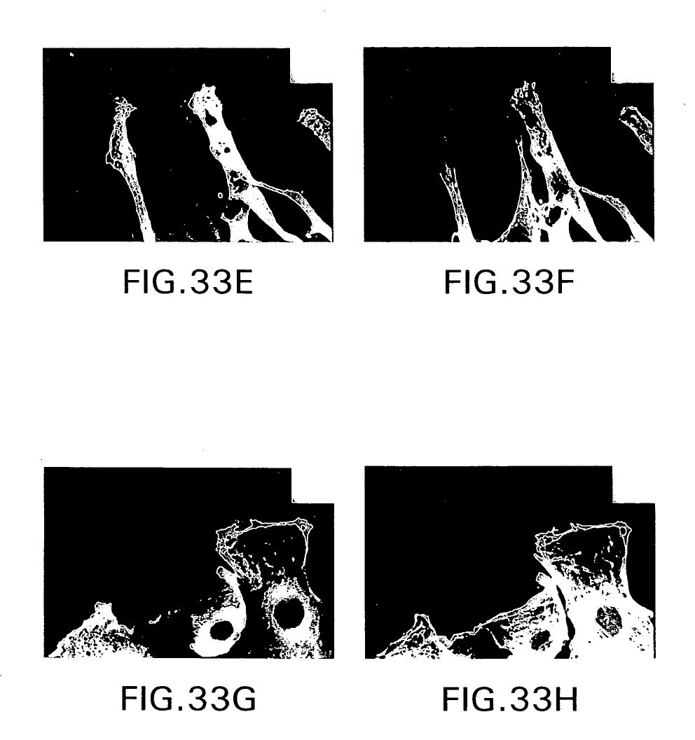

FIG. 33A-H depict the result of cell-wounding experiments, in which SSeCKS, but not actin, is associated with the growing edge of a cell following injury. Immediately after injury to the monolayer, SSeCKS was present throughout the cell (FIGS. 33A and C) and actin stress fibers were apparent (FIGS. 33B and D). In contrast, the growing edge of peripheral cells 5 hours after injury was enriched with SSeCKS (FIGS. 33E and G) but devoid of actin stress fibers (FIGS. 33F and H).

### 12. EXAMPLE

### SSeCKS and the Transformed Phenotype

### 12.1. Materials and Methods

Cell lines: NIH3T3 fibroblast and Ωe packaging cells (Morgenstern and Land, 1990, Nucl. Acids Res. 18:3587-3596) were grown in DMEM (Dulbecco's modified Eagle's media) containing 10% heat-inactivated calf serum (GIBCO), 100 units/ml of penicillin, 100 units/ml of streptomycin and 250 ng/ml of amphotericin B (Fungizone) (GIBCO). S2-6/S24 is a tetracycline-regulated SSeCKS overexpressing cell line. S2-6/S24 cells were cultured in histidine deficient DME (Irvine Scientific) supplemented with 0.5 mM L-histidinol (Sigma), calf serum, penicillin/streptomycin/amphotericin B, 2 mg/ml of puromycin and 0.5 mg/ml of tetracycline (Sigma).

Generation of temperature-sensitive src expressing cell lines: pLJ/ts72src and pLJ retroviral vectors, harboring a neomycin-resistant gene (neoR), were independently introduced into Ωe packaging cells using the calcium phosphate precipitation method. Supernatants were harvested after 24 h and used to infect S2-6/S24 cells in the-presence of 8 mg/ml of polybrene (Sigma). Following G418 (400 mg/ml) (GIBCO) selection at 35° C. (permissive temperature=PT), individual G418-resistant, morphologically transformed colonies were expanded and maintained in G418 media at 35° C. Clones were selected which were morphologically transformed at the PT but untransformed after 2-3 days growth at the non-permissive temperature (NPT=39.5 C).

Proliferation assay: 10⁴ cells were seeded into 24-well plates, and the next day cells were trypsinized and counted to establish a baseline plating efficiency. The remaining cells were grown in the presence or absence of tetracycline at the PT or NPT, and counted every two days using a hemacytometer (Fisher).

Transformation assays: 1 mg of pMv-src plasmid DNA (Johnson et al., 1985, Mol. Cell. Biol. 5:1073-1083) and 10 mg of SSeCKS/p Babehygro or 10 mg of pBabehygro ((Morgenstern and Land, 1990, Nucl. Acids Res. 18:3587-3596) were co-transfected into NIH3T3 cells using the calcium phosphate precipitation method. Foci were counted following fixation with methanol and staining with 0.4% crystal violet 18 days after transfection. To control for DNA transfection delivery, aliquots of the cells were grown in the presence of 85 mg/ml of hygromycin, and the number of hygromycin-resistant colonies were scored after 2 weeks of growth. Colony formation in soft agar was performed as follows. Two days after co-transfection, 1-20×10⁸ cells were mixed with top agar (0.4%) components supplemented with 85 mg/ml of hygromycin and cultured for 3 weeks. The colony number was determined by incubating the cells for 2 days with 40% INT staining solution (Sigma). To compare the anchorage-independent growth of S24/ts72src to S24/pLJ, 10⁵ cells were mixed with top agar in the presence or absence of tetracycline and then grown for 3 weeks at the PT or the NPT.

In vitro invasion assay: Matrigel-coated polycarbonate filters were used to measure the invasive ability of S24/ts72src cells under various conditions. 10⁵ cells grown in the presence or absence of tetracycline for 4 days were seeded onto the matrigel-coated filters in Boyden chambers (in 0.2 ml of serum-free media). The bottom wells contained 0.5 ml of media containing 10% CS and 10% conditioned media from NIH/v-src cells. The cells were incubated in the presence or absence of tetracycline at the PT or NPT for 48 h. The non-invasive cells were removed from the upper chamber with a cotton swab and the invasive cells identified by fixing with methanol and staining with Giemsa.

Western blotting assays: Cells grown in the presence or absence of tetracycline for 4 days at the PT or NPT were washed thrice with ice-cold PBS (phosphate buffered saline), lysed with 0.5 ml of RIPA buffer containing 150 mM NaCl (Gelman et al., 1993, Oncogene, 8:2995-3004) per 10-cm plate. Protein levels were normalized using Bio-Rad Protein Assay (Bio-Rad Laboratories). Protein aliquots normalized for protein content (Pierce kit) were electrophoresed through SDS-polyacrylamide gels and then electrophoretically transferred to Immobilon-P (Millipore Corporation). Non-wetted membranes were incubated with rabbit antibody against SSeCKS, rabbit antibody against ERK2 (Santa Cruz), mouse monoclonal EC10 specific for avain SRC (Parsons et al., 1984, J. Virol. 51:272-282), and mouse monoclonal PY20 specific for phosphotyrosine (Transduction Labs), respectively. Alkaline phosphatase-labeled sheep anti-rabbit IgG or goat anti-mouse IgG (Boehringer Mannheim) were used as secondary antibodies. The blot was visualized by adding the substrate solution, Western Blue (Promega).

Kinase assays: Lysates containing 200 mg to 400 mg of protein were immunoprecipitated with rabbit anti-SRC (East-Acres Biologicals, southbridge, Mass.) or rabbit anti-ERK2 antibodies, which were pre-bound to Affi-Prep protein A beads (Bio-Rad Laboratories). SRC kinase activity was performed in 50 mM Tris pH8.0/10 mM MnCl2 containing 10 mCi of δ³²P[ATP] for 10 min at room temperature as described in Gelman et al., 1993, Oncogene, 8:2995-3004. ERK2 kinase activity was performed in 10 mM HEPES buffer pH7.0, 10 mM magnesium acetate bearing 50 mM of ATP, 10 mCi of δ³²P[ATP], and 40 mg of MBP (Myelin Basic Protein) (GIBC\dot{O}) for 30 min at 30° C. In order to monitor JNK activity, lysates containing 400 mg of protein were incubated with 50 mg of GST-JUN-glutathione-sepharose beads. After washing, kinase activity was assayed in 50 mM Tris pH8.0, 5 mM MnCl2, 5 mM MgCl2, 10 mCi of δ³²P[ATP] for 10 min at room temperature. The reaction mixtures were boiled and electrophoresed through a SDS-polyacrylamide gel, followed by autoradiography.

Gelatin zymography; S24/ts72src or S24/pLJ cells were cultured in the presence or absence of tetracycline for 4 days at the PT or NPT and then grown in serum-free medium for 24 h. Aliquots of conditioned media, normalized for cell number, were applied directly to 10% SDS-polyacrylamide gel containing 1 mg/ml of gelatin (Sigma). Following electrophoresis, the gel was incubated with 2.5% Triton X-100 for 30 min at room temperature to remove SDS. Then the gel was incubated with 50 mM Tris-HCl (pH7.7) containing 5 mM CaCl2 and 0.5% NaN3 for 40 h at 37° C. Gels were stained with Brilliant Blue G-250 for 20 min, and destained to visualize clear bands representing collagenase and gelatinase activity.

Indirect immunofluorescence: S24/ts72src or S24/pLJ cells cultured in the presence or absence of tetracycline at the PT or NPT for 4 days were fixed and stained as described in Gelman and Silverstein, 1986, J. Mol. Biol. 191:395-409, using a 1:250 dilution of immunoaffinity-purified rabbit anti-SSeCKS (Lin et al., 1995, Mol. Cell. Biol. 15:2754-2762) followed by 1:250 FITC-labeled goat anti-rabbit Ig (Boerhinger Mannheim) and a 1:800 dilution of TRITC-phalloidin (Sigma).

### 12.2 Results

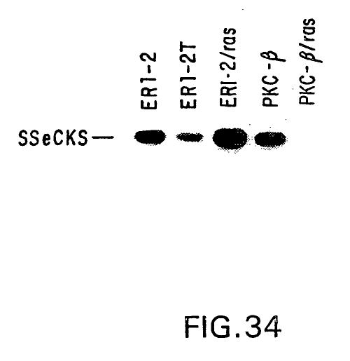

Correlation of SSeCKS transcriptional down regulation with anchorage-independent growth. Although SSeCKS is transcriptionally down regulated by src and ras (Lin et al., 1995, Mol. Cell. Biol. 15:2754-2762), it is unclear which parameter of in vitro transformation correlates with the down regulation. Thus, we probed a panel of ras-transformed and revertant Rat-6 cells (Feinleib and Krauss, 1996, Molec. Carcinog. 16:139-148) for steady state SseCKS RNA expression. FIG. 34 shows that transformation by ras leads to greater than 15-fold suppression in SSeCKS RNA levels compared to the untransformed Rat-6/PKC β1/ras cells. Most significantly, SSeCKS down regulation was detected in ER1-2T cells, transformed revertants of ER1 -2 which grow in soft agar, but not detected in ER1-2/ras cells, which exhibit increased refractility but no growth in soft agar. These data indicate that SSeCKS expression strongly correlates with contact inhibited growth.

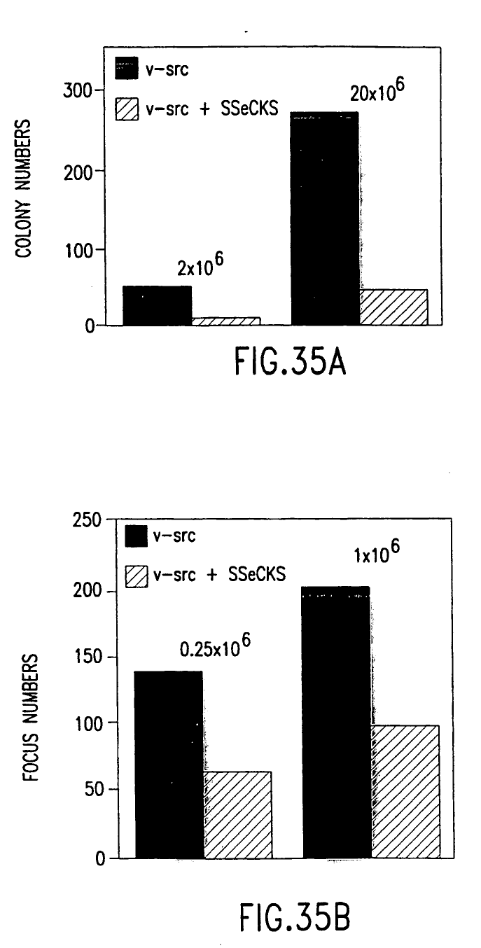

SSeCKS inhibits src-induced soft-agar colony formation. Transfection of v-src expression plasmids induced anchorage-independent growth of rodent fibroblasts (FIG. 35), as demonstrated in Johnson et al., 1985, Mol. Cell. Biol 5:1073-1083. Co-transfection of molar gene equivalents of an SSeCKS expression plasmid with pMv-src resulted in a 6-to 30-fold decrease in soft agar colony formation over vector controls (FIG. 35). SSeCKS also decreased src-induced focus formation 2-fold (this suppression level is probably less than in the soft agar colon assay because there was no selection on hygromycin). The differences in colony forming efficiency was not due to differences in delivery of DNA as equal aliquots of pMv-src-and pMv-src/pSSeCKS-transfected cells gave similar numbers of hygromycin resistant colonies after 2 weeks of selection.

We failed on multiple occasions to produce NIH3T3, Rat-6, or 3Y1 cell lines with stable, constitutive SSeCKS expression using retrovirus infection or transfection. All attempts resulted in a similar phenomenon. (Lin et al., 1995, Mol. Cell. Biol. 15:2754-2762), namely that the SSeCKS colonies did not readily proliferate past 20-50 cells and the few clones that expanded slowly after 2 months of selection showed deletions of the transduced SSeCKS cDNA by southern blot analysis. This indicates that the constitutive overexpression of SSeCKS in these rodent fibroblasts is antagonistic to sustained proliferation.

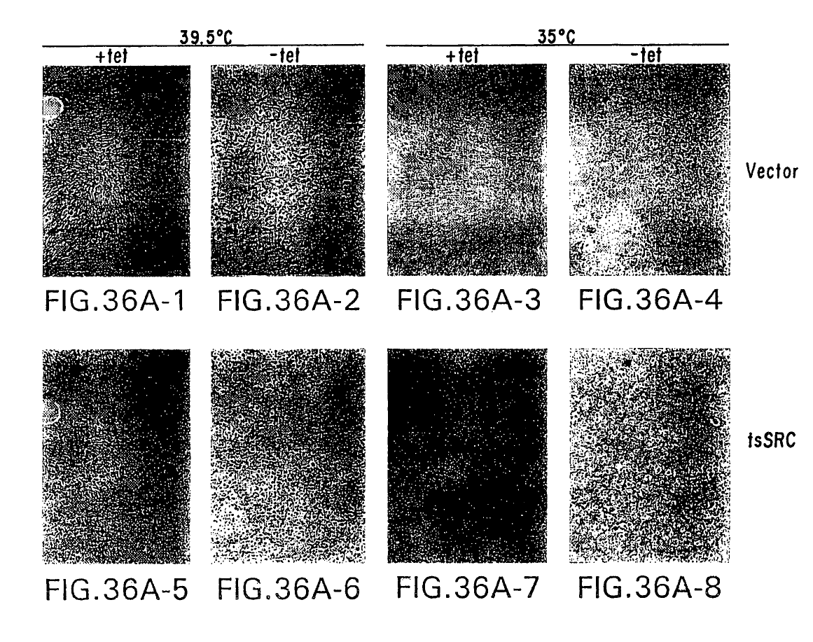

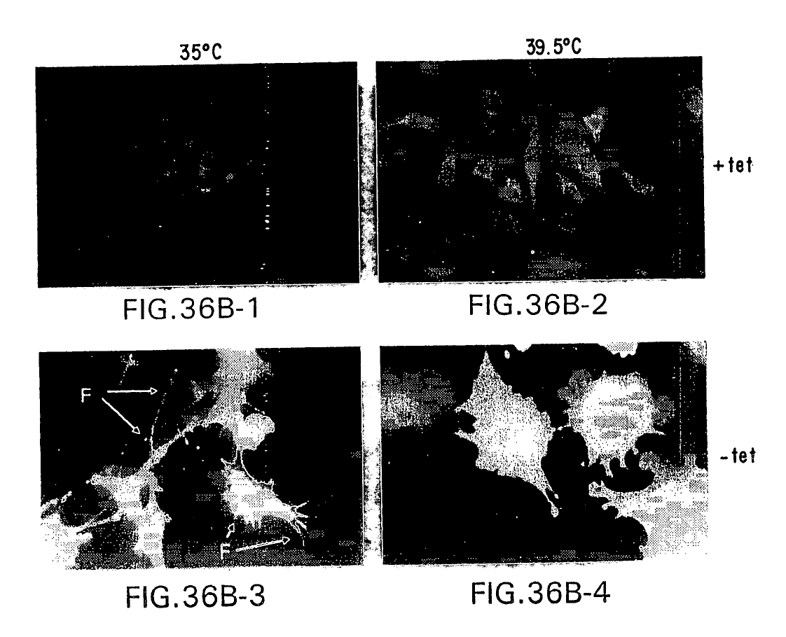

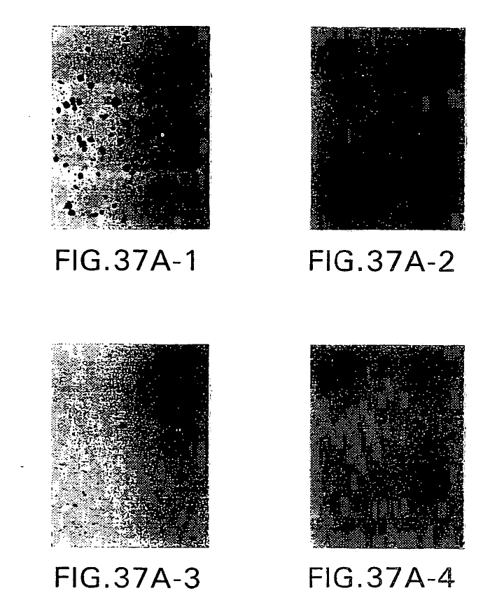

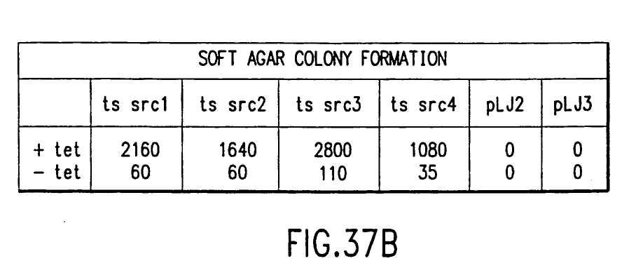

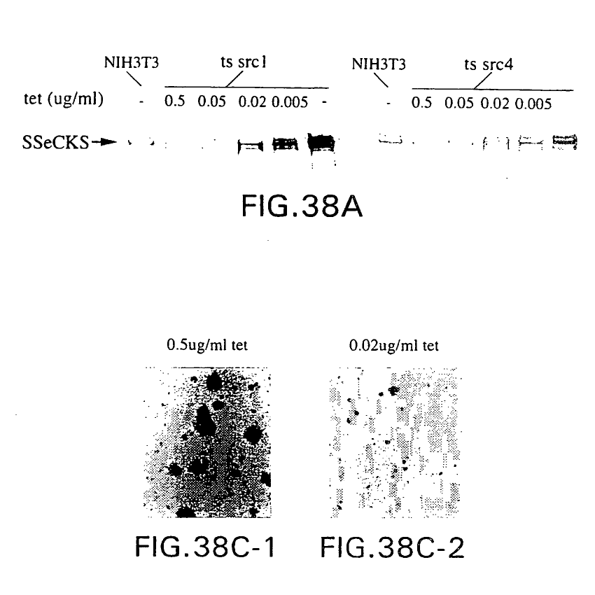

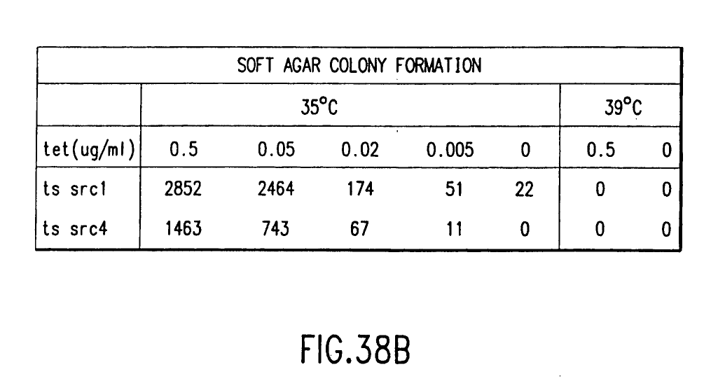

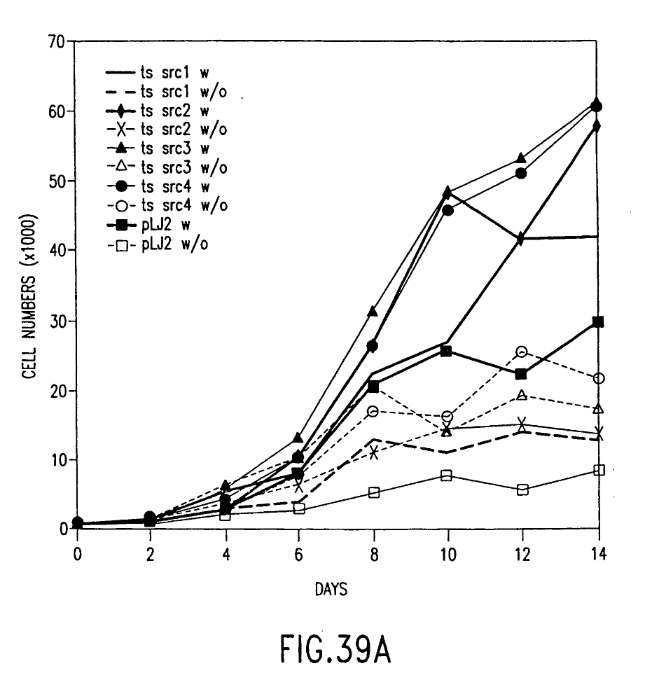

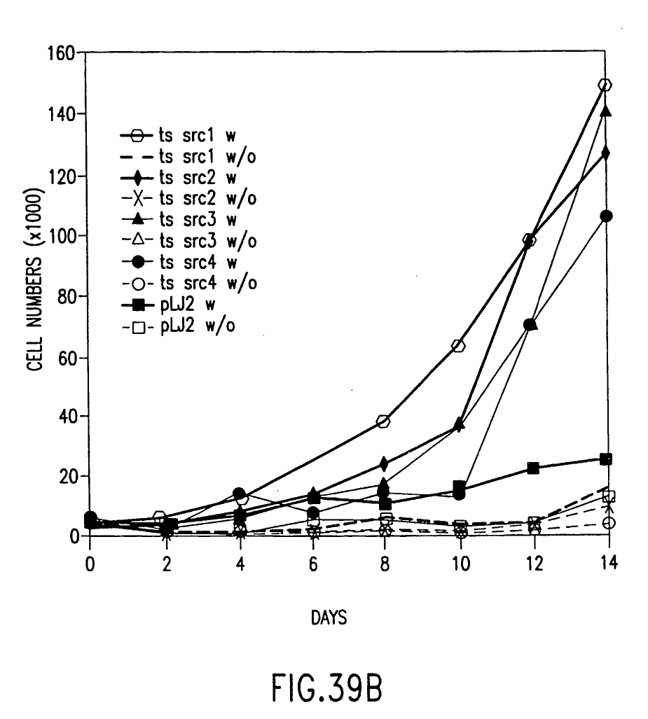

Cell lines with conditional SSeCKS and v-src expression (S24/ts72src). The suppression of src-induced anchorage independent growth by SSeCKS may be due to induction of growth arrest or to the selective loss of tumorigenic parameters in the absence of growth arrest. To differentiate between these two possible effects, NIH3T3 cell lines were developed with tetracycline regulated SSeCKS expression and conditional transformation with ts72src as described in Materials and Methods. In the absence of ectopic SSeCKS expression (+tetracycline), the cells were untransformed at the NPT (39.5° C.). At the PT (35° C.), however, the cells were transformed as shown by increased refractility, growth in soft agar, focus formation, and proliferation in low (0.5%) and high (10%) serum conditions (FIGS. 36, 37 and 39, Table 2). SSeCKS expression (−tetracycline) at the NPT caused increased cell flattening (FIG. 36B) and decreased cell proliferation (Table 2). This agrees with the result that the inducible overexpression of SSeCKS causes growth arrest of untransformed rodent fibroblasts in G1. At the PT, SSeCKS expression inhibited v-src-induced parameters of morphological transformation and tumorigenesis (Table 2; FIG. 36) without inhibiting proliferation rates in high serum conditions (FIG. 39A). Specifically, SSeCKS caused decreased refractility (FIG. 36A), increased cell flattening (FIG. 36B), a 5- to 10-fold decrease in the number and size of colonies in soft agar (FIGS. 37 and 38) and a 15-fold decrease in proliferation rates in low (0.5%) serum (FIG. 39B). The SSeCKS-induced effects are detected in several independently isolated S24/ts72src cell lines, and thus, do not reflect the idiosyncracies of a single clone. These data demonstrate a tumor suppressive activity for SSeCKS which is independent of affects on proliferation. The data also indicate that v-src induces mitogenic signals not inhibited by SSeCKS during the process of tumor suppression.

It could be argued that the apparent tumor suppression may be due to the gross overexpression of SSeCKS in our 24/ts72src cells at the PT (greater than 50-fold over background after removal of tetracycline). However, by varying the concentration of tetracycline, we re-expressed SSeCKS to “normal” levels (as in the parental NIH3T3 cells) and still demonstrated a 10-12 fold decrease in src-induced soft agar colony formation (FIG. 38). Thus, the SSeCKS′ tumor suppressive effect is not due to overexpression or non-specific toxicity.

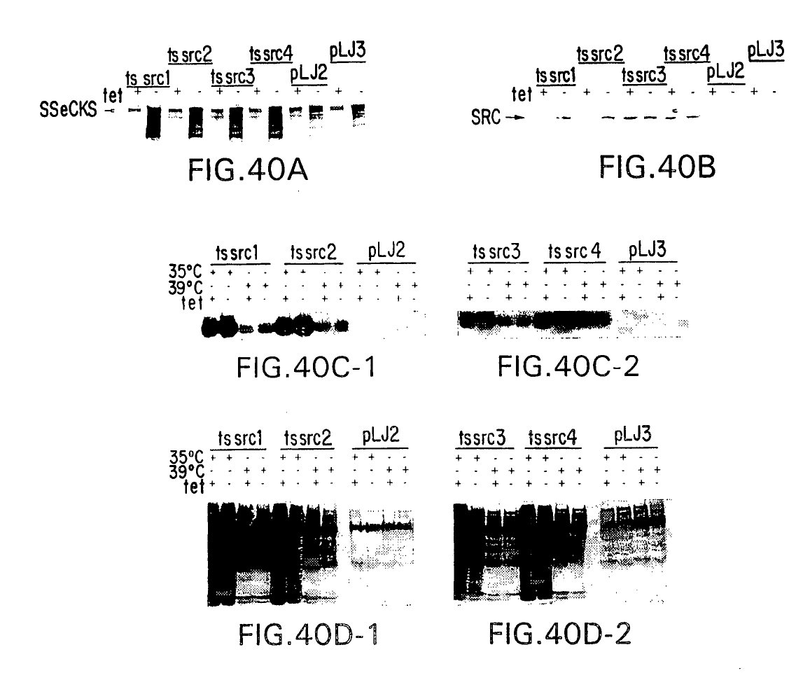

p60v-src and SSeCKS expression in S24/ts72src cells. FIG. 40A shows that removal of tetracycline results in a more than 20-fold increase in the levels of SSeCKS protein (280/290 kDa doublet and 240 kDa forms) in S24/ts72src cell lines in comparison to control lines with vector alone. SSeCKS levels begin to rise 8 hours after removal of tetracycline and are maximal 64-72 hours later. We have observed previously several other bands in overloaded gels recognized by anti-SSeCKS sera including 140, 95, 65 and 44 kDa species. The increased abundance of these species in FIG. 40A in the absence of tetracycline strongly suggests that they are primary products of the SSeCKS cDNA or are cleavage products of the larger SSeCKS forms, as opposed to other cellular proteins with shared epitopes. It is unlikely that they are breakdown products as a [³⁵S]-methionine label cannot be chased from the 280/290 or 240 kDa forms to the smaller forms.

The levels of transduced ts72src protein were unaltered by SSeCKS induction (FIG. 40B). The in vitro kinase activity of p60ts72src isolated at the PT was 50-fold greater than the activity of the protein isolated at the NPT (FIG. 40C) in agreement with Moroney et al., 1992, Oncogene 7:1207-1214). SSeCKS slightly increased (2-3 fold) p60ts72src kinase activity at the PT but had not effect at the NPT (FIG. 40C). SseCKS expression did not grossly decrease the in vivo level of number of the src-induced phosphotyrosine substrates in cells grown at the PT (FIG. 40D). These data indicate that the tumor suppressive effects of SseCKS are not manifested through the inhibition of v-src expression or kinase activity.

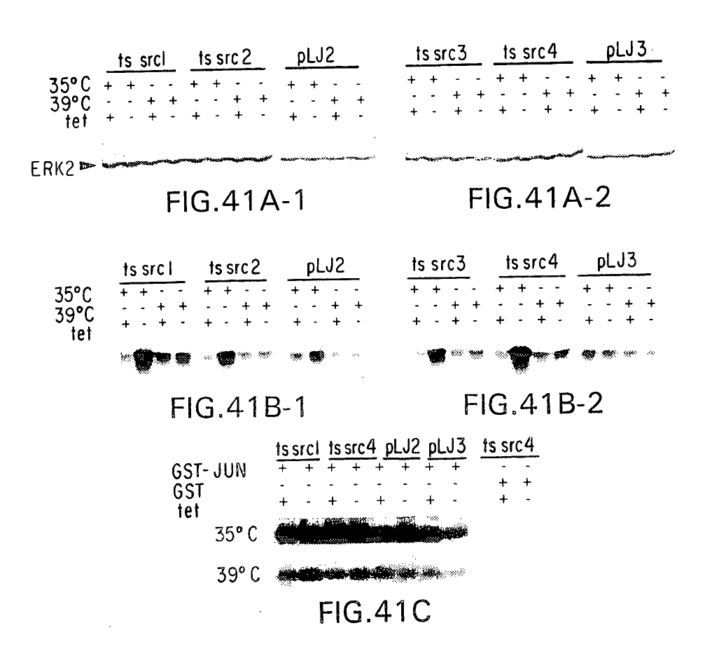

Effects of SSeCKS on ERK and JNK activities. Mitogenic pathways in fibroblasts are controlled by ERK- and JNK-regulated signals (Serijard et al., 1994, Cell 76:1025-1037; Minden et al., 1994, Science 266:1719-1723). SSeCKS expression did not change the level of ERK2 (FIG. 41A). However, the in vitro kinase activity of ERK2 was unexpectedly increased 5- to 10-fold in all four S24/ts72src clones in response to SSeCKS induction at the PT only (FIG. 41B). ERK2 activity was not constitutively activated in S24/ts72src cell lines kept at the PT, consistent with the notion that v-src does not constitutively activate ERK in long-term transformed cultures. Finally, JNK activity was unaffected by SSeCKS expression, but was increased 3-10 fold at the PT in comparison to the NPT (FIG. 41C). These data indicate that the tumor suppressive effects of SseCKS are not due to the inhibition of ERK2 and JNK activities. Thus, the ability of SSeCKS to induce growth arrest in untransformed cells at the NPT is dominant over the ability of ERK and JNK to induce proliferation. It is unclear whether the lack of growth arrest during SSeCKS expression at the PT relates to the increased ERK2 activity.

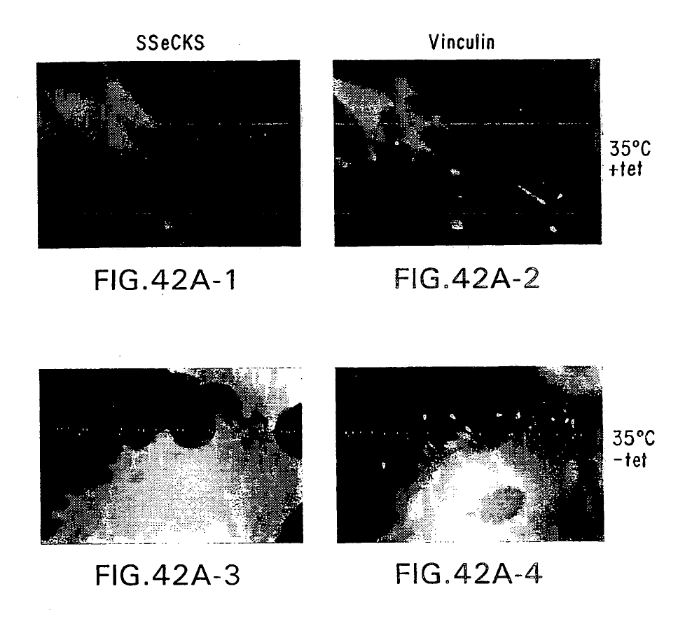

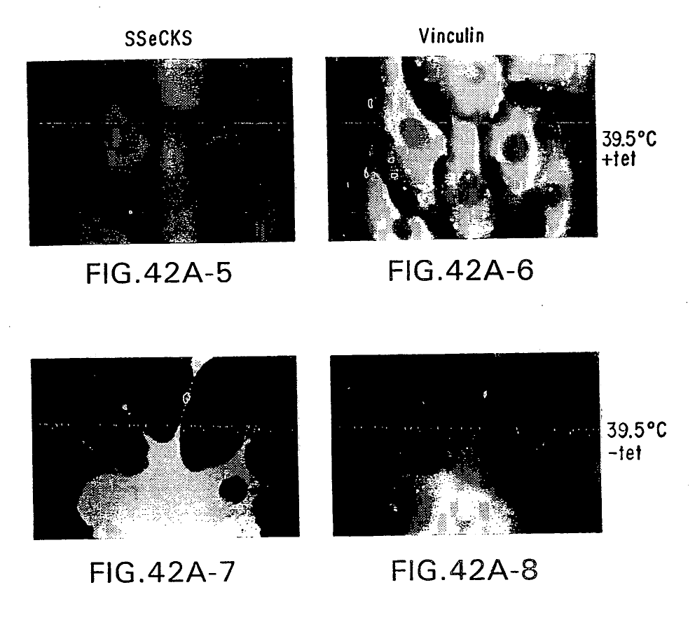

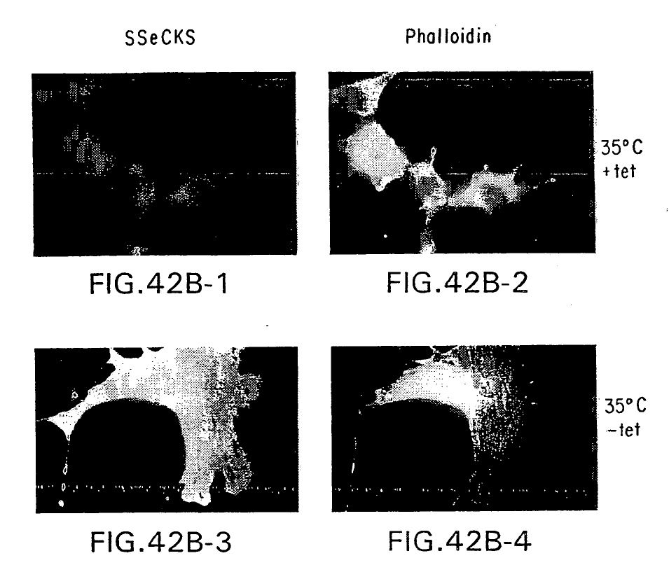

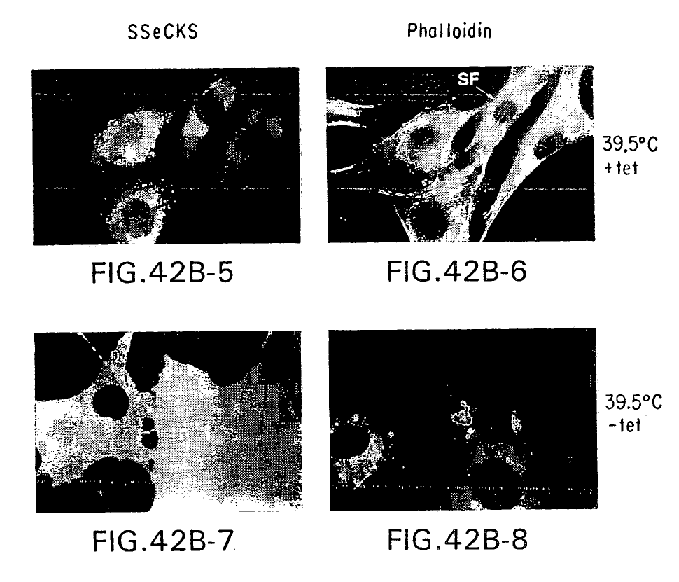

Effect of SseCKS expression of cytoskeletal architecture. A feature of src-induced morphological transformation is a loss of actin-based stress fibers and adhesion plaques, and the resulting loss of cytokinetic structures such as lamellipodia and filopodia (Jove and Hanafusa, 1987, Ann Rev. Cell Biol. 3:31-56; Lo and Chen, 1994, Cancer Metastasis Rev. 13:9-24). This correlates with increased cell rounding (refractility), loss of integrin-mediated adhesion, and loss of contact inhibited growth. Shifting of S24/ts72src cells from the NPT to the PT in the absence of SSeCKS expression resulted in the loss of vinculin-associated adhesion plaques and actin stress fibers coincident with increased cell refractility (FIGS. 42A and 42B). The transformed cells exhibited prominent actin staining at the cell edge in contrast to untransformed cells where actin stress fibers were prominent in the cell's center (FIG. 42B). S24/ts72src cells grown at the NPT with SSeCKS expression are extremely flat and devoid of stress fibers and adhesion plaque structures (FIGS. 42A and 42B). As the steady state levels of total cellular actin and vinculin are unchanged by SSeCKS overexpression in S24 cells, this effect is likely manifested by a re-organization of cytoplasmic components rather than changes in de novo synthesis. In contrast, S24/ts72src cells grown at the PT with SSeCKS expression are only slightly flatter than control cells, yet they exhibit a more normal array of cytoskeletal and cytokinetic structures. Specifically, these cells have increased production of filopodia- and lamellipodia-like structures as well as vinculin-associated adhesion plaques and stress fibers (FIGS. 42A and 42B), although the stress fibers are smaller than in control cells. The filopodia-like structures induced by SSeCKS are often extremely elongated, showing a bead-like periodicity of SSeCKS staining. These structures are not true filopodia as they lack actin fibers and vinculin. As many of these structures stain heavily at their bases for F-actin and vinculin, it is likely they represent herniations at the sites of focal adhesions. These data strongly suggest that SSeCKS suppresses tumorigenicity by controlling cytoskeletal architecture.

Induction of apoptosis. The over expression of certain tumor suppressor genes such as p53 can induce growth arrest in untransformed cells or apoptosis in cells expressing specific oncogenes (Hinds and Weinberg, 1994, Curr. Opin. Genet. Dev. 4:135-141). Having shown that SSeCKS expression causes growth arrest of S24/ts72src cells at the PT, we determined whether SSeCKS could increase the apoptotic index following src activation. The activation of src in the absence of SSeCKS expression marginally increased the level of apoptosis over controls (5.5 to 11.1%), which parallels the increased apoptotic index of transformed cells described by Hoffman and Lieberman, 1994, Oncogene 9:1807-1812. However, the level of apoptosis was unchanged by SSeCKS expression at the PT. Thus, any direct role for SSeCKS in controlling proliferation is likely via the regulation of the cytoskeleton rather than through control of the cell cycle.

Effect of SSeCKS on invasiveness. The results in FIGS. 34, 35, 37 and 38 indicate that SSeCKS is involved in the control of anchorage-independent growth. We extended this analysis to determine SSeCKS′ affect on the invasiveness using Matrigel-coated Boyden chambers. Table 3 shows that SSeCKS expression significantly decreased the invasive potential of S24/ts72src cells at the PT.

Effects of SSeCKS on production of invasion enzymes. The metastatic potential of tumors in vivo as well as in vitro parameters of transformed growth by fibroblasts such as colon formation in soft-agar correlates with the induction of metalloproteinases (Aznavoorian et al., 1993, Cancer 71:1368-1383; Mignatti and Rifkin, 1993, Physiol. Revs. 73:161-195). We used zymography to assay for collagenase and gelatinase activity in the media of S24/ts72src cells grown at the PT or NPT in the presence or absence of SSeCKS expression. SSeCKS did not alter the secreted level of the 72 kDa collagenase and the 55 kDa gelatinase at either the PT or NPT. Thus, the decreased invasiveness detected in Table 2 is manifested by enzymes other than these metalloproteinases.

Evidence presented here indicates that SSeCKS suppresses many parameters of v-src-induced in vitro neoplastic behavior without inducing growth arrest. Taken with data of SSeCKS down-regulation in breast cancer lines, there is strong evidence to define SSeCKS as a novel Type II tumor suppressor. Our data indicate that SSeCKS overexpression alters cytoskeletal architecture and cell signaling resulting in either growth arrest in untransformed cells or tumor suppression of v-src-induced cells. It is unlikely that SSeCKS overexpression is simply toxic as S24/ts72src cells proliferate normally at the PT without tetracycline.

SSeCKS down-regulation correlates best with anchorage-independent growth rather than increased refractility. This is chiefly based on the selective suppression of SSeCKS transcript levels in cells showing transformed morphology and growth in soft agar. Thus, SSeCKS transcription is not down-regulated in ER1 -2/ras cells, which do not grow in soft agar but do exhibit a rounded transformed morphology. It should be stressed that these cells are not as refractile as the fully transformed Rat-6/ras, and thus, it remains to be determined whether they retain cytoskeletal structures typical of untransformed cells. Nonetheless, overexpression of SSeCKS inj S24/ts72src cells at the PT suppresses tumorigenic phenotypes in addition to growth in soft agar, such as increased refractility, focus formation and growth in low serum, indicating that SSeCKS affects multiple structural and signaling pathways. Most importantly, these data show that SSeCKS expression tracks with transformation such that normal levels of SSeCKS RNA are re-established in untransformed, flat revertants of Rat-6/ras cells.

The ability of SSeCKS to inhibit src-induced soft agar colony formation cannot be due to toxic overexpression as the controlled re-expression of SSeCKS (by varying tetracycline concentrations) also decreases colony formation at least 10-fold. Work by Ingber suggests that transformed cells retain nucleation complexes for structural integrity, yet exhibit altered cell morphologies due to an increased turnover of these complexes (Wang and Ingber, 1994, Biophys J. 66:2181-2189). This suggests that slight changes in the levels of cytoskeletal proteins such as SSeCKS are sufficient to re-establish normal controls on cytoskeletal architecture, most likely by changing the turnover rate of the structural complexes. SSeCKS-induced tumor suppression is not due to the loss of tssrc expression, in vitro or in vivo src kinase activity or src-enhanced JNK activity. In contrast, SSeCKS overexpression enhances src-induced ERK2 specific activity 5-10 fold. It is possible that this increase is responsible for the ability of S24/ts72 src cells to proliferate at the PT, whereas in the absence of this increase at the NPT, cells are growth-arrested. Alternatively, v-src may induce a proliferation pathway not inhibited by SSeCKS, such as the recently described STAT pathways (Yu et al., 1995, Science 269:81-83).

It is highly likely that SSeCKS directly induces the cell flattening and coincident loss of actin stress fibers and vinculin-associated adhesion plaques we observe following removal of tetracycline at the PT and NPT. This is because i) SSeCKS physically associates with the cytoskeletal (cortical) matrix and ii) cells begin to flatten and stress fibers begin to be marginalized away from he cell's center simultaneous with the appearance of induced SSeCKS levels after tetracycline removal. Recent technical improvements have helped demonstrate the complex, intertwined nature of a multi-fibrous cytoskeletal matrix (Penman, 1995, Proc. Natl. Acad. Sci U.S.A. 92:5251-5257). This concept suggests that re-establishment of SSeCKS in the matrix is sufficient to induce the re-modeling of other cytoskeletal networks.

SSeCKS′ affect on the cytoskeleton may intersect with src's control of cellular morphology mediated by FAK. FAK is activated by tyrosine phosphorylation mediated by src family kinases (Zachary and Rozengurt, 1992, Cell 71:891-894; Ridley and Hall, 1994, EMBO J. 13:2600-2610), and is associated with the turnover of adhesion plaques and the loss of stress fibers (IIIc et al., 1995, Nature 377:539-544; Ridley and Hall, 1994, EMBO J. 13:2600-2610). However, the loss of stress fibers induced by the GTPase Rho precedes FAK activation, as FAK is not tyrosine phosphorylated by constitutively activated Rho (N14) in the presence of cytochalasin D (Ridley and Hall, 1994, EMBO J. 13:2600-2610). Thus, it is plausible that expression of SSeCKS may block src-mediated FAK activation by causing the reorganization of the cytoskeletal matrix or by inhibiting tyrosine phosphorylation of FAK. Our finding that SSeCKS enhances src-induced activation of ERK2 kinase activity indicates that SSeCKS affects signaling pathways. As ERK2 and other signaling mediators are enriched in focal adhesion complexes following the engagement of integrins to their extracellular ligands (Miyamoto et al., 1995, J. Cell Biol. 131:791-805), SSeCKS may affect ERK2 activity by altering the cytoskeletal components in these sites or by preventing the turnover of these complexes.

There is growing precedence that the re-establishment of cytoskeletal architecture can override oncogene-induced parameters of tumorigeneiss and apoptosis (Welsh et al., 1993, J. Cell Physiol. 17:155-158; Frisch and Francis, 1994, J. Cell. Biol. 124:619-626; Lo et al., 1994, Bioessays 16:817-823). Based on the tensegrity model of Ingber (Ingber et al., 1994, Rev. Cytol. 150:173-224), changes in structural integrity directly affect the shape of the nucleus, and therefore, nuclear events such as transcription. These data underscore the emerging importance of cytoskeletal architecture in controlling mitogenic and proliferative signal pathways. Interestingly, other tumor suppressors C especially those that directly mediate cell cycle control e.g.—p53, Rb, Mxi 1) C often induce growth arrest and cell flattening when ectopically overexpressed (Hinds and Weinberg, 1994, Curr. Opin. Genet. Dev. 4:135-141; Hinds et al., 1992, Cell 70:993-1006). In these cases, the cellular effects lag behind the expression of the tumor suppressors by at least 1 day, and thus, may be considered indirect effects.

TABLE 2
Growth Characteristics of S24/ts 72src cells^{a} at
the non-permissive temperature (NPT) or permissive
temperature (PT) for v-src kinase activity.

| | NPT (39.5° C.) | PT (35° C.) |
|---|---|---|
| +tetracyline | flat | very transformed |
| | soft agar:^{b} −/− | soft agar: ++++ |
| | foci:^{c} −/+ | foci: ++++ |
| | 10% CS: +++ | 10% CS: ++++ |
| | 0.5% CS: −/+ | 0.5% CS: ++ |
| −tetracycline | very flat | flat |
| | soft agar: −/+ | soft agar: −/+ |
| | foci: −/+ | foci: −/+ |
| | 10% CS: + | 10% CS: ++++^{d} |
| | 0.5% CS: −/− | 0.5% CS: −/− |

^{a}Cell lines containing tetracycline-regulated SSeCKS expression (S24) infected with packaged retovirus encoding ts 72v-src, as described in Materials and Methods. Results are common for S24/ts72src clones 1-4.
^{b}Colony formation in soft agar as described in FIG. 37.
^{c}Focus formation assays were performed as described previously (Gelman et al., 1993, Oncogene 8: 2995-3004).
^{d}Lower saturation density when compared with +tet/35° C.

TABLE 3
In vitro invasiveness of S24/ts 72 src cells in Matrigel^{a}

| | +tet/39.5° C. | +tet/35° C. | −tet/35° C. |
|---|---|---|---|
| S24/ts 72src, clone 1 | 0 | 72 +/− 3 | 0 |
| S24/ts 72src, clone 2 | 0 | 9 +/− 1 | 0 |
| S24/ts 72src, clone 3 | N.D. | 1850 +/− 20 | 0 |
| S24/vector, clone 2 | N.D. | 1 | 2 +/− 1 |

^{a}10⁵ cells were located onto uncoated (control) Boyden chambers or those coated with Matrigel (Collaborative Biochemicals), incubated and processed as described in Materials and Methods. The cell counts in control chambers varied less than 10%.

Various publications are cited herein, which are hereby incorporated by reference in their entireties.

### 13. EXAMPLE

### Loss of SSeCKS/Gravin Correlates with the Metastatic Potential of Cancer Cells

The data presented below shows that human SSeCKS, herein referred to as gravin, maps to a single chromosomal site, 6q24-25.2, a hot spot for deletion in advanced prostate cancer. In addition, reexpression of SSeCKS in MLL cancer cells suppresses cell rounding, and the generation of secondary lung metastases in nude mice.

### 13.1. Materials and Methods

### 13.1.1 Cell Culture

MatLyLu (MLL) cells, EP12 (EPYP-1; a gift of K. Pienta, U. of Michigan Comprehensive Cancer Center), LNCaP/tTA (LNGK9; a gift of T. Powell, Memorial-Sloan Kettering Cancer Center; ref. 23), and HeLa (ATCC #CCL2.1) were grown in DME (GIBCO; Gaithersburg, Md.) supplemented with 10% fCS (GIBCO). P69 (P69SV40T), M2182 and M12 (gifts of J. Ware, Medical College of Virginia) were grown in RPMI 1640 plus insulin, transferrin and selenium (Collaborative Biochemicals), dexamethasone (Sigma; St. Louis, Mo.) and EGF (Collaborative) as described (Jackson et al., 1996, Cytogenetic 87:14-23).

### 13.1.2 Production of Tetracycline Regulated MLL Cell Lines

Production of tetracycline-regulated MLL cell lines: MLL/tTAK cells, expressing a tetracycline (tet)-regulated tTA transactivator (Shockett et al., 1995, Proc. Natl. Acad. Sci., USA 92:6522-6526), were produced by transfecting with CaPO₄/DNA precipitates containing 3.5 μg of pTet-tTAK and 0.6 μg of pRSV/hygro followed by selection of stable transfectants in 400 μg/ml of hygromycin (Sigma). Individual clones were tested for the ability to induce expression of tet_{O}/luciferase (pUHD13-3) in the absence of tet (Gossen et al., 1992, Proc. Natl. Acad. Sci., USA 89:5547-5551). Clones 2 and 7 were chosen for secondary transfection with 3.8 μg of pUHD10-3/SSeCKS (Gelman et al., 1998, Cytoskelet. 41:1-17) and 1.6 μg of pBABE/puro (Morgenstern et al., 1990, Nucleic Acids Res. 18:3587-3596), and stable transfectants were isolated after selection in hygromycin and puromycin (8 μg/ml). All cells were selected in 5 μg/ml tet and then maintained on 0.7 μg/ml.

### 13.1.3 Fish Analysis

FISH analysis was performed by See DNA Biotech, Inc. (Downsview, Ontario). A 6.2 kb Gravin cDNA fragment was labeled with Biotin-14-dATP using a BRL BioNick kit according to the manufacturer's specifications. Slides were prepared with human lymphocytes grown in ax-minimal essential medium containing 10% FCS, phytohemagglutinin, and bromodeoxyuridine (180 μg/ml; Sigma), then grown for 6 h in medium containing thymidine (2.5 μg/ml). FISH detection was performed as described previously (Heng et al., 1992, Proc. Natl. Acad. Sci. USA 89:9509-9513; Heng et al., 1993, Chromosoma (Berl.) 102:325-332). Among 100 mitotic figures that were checked, 81 showed signals on one pair of the chromosomes (i.e.—81% hybridization efficiency). DAPI banding patterns mapped the signals to the long arm of chromosome 6, and based on the summary from 10 independent photographs, Gravin was mapped by higher resolution to 6q24-25.2. No additional loci were identified by FISH detection, suggesting the absence of highly conserved gene family members.

### 13.1.4 Colony Assay in Soft Agar

10⁴ cells were plated into soft agar in 6 cm wells as described previously (10) and then grown for 3 weeks at 37° C. with feedings of fresh media twice/week.

### 13.1.5 Northern and Western Blotting

Total or poly (A)-selected RNAs were electrophoresed, blotted and probed with [³²P]-rat SSeCKS cDNA as described (Lin et al., 1995, Mol. Cell. Biol., 15:2754-2762). RIPA lysates containing 40-100 μg of total protein were prepared and immunoblotted using rabbit polyclonal anti-SSeCKS Ig as described (Lin et al., 1996, J. Biol. Chem. 271:28,430-28,438), using either alkaline phosphatase- or horseradish peroxidase-labeled secondary antibodies followed by Western Blue substrate (Promega; Madison, Wis.) or ECL (New England Nuclear; Boston, Mass.) for visual or chemiluminescence detection, respectively. Images were scanned on an Agfa Duoscan T1200, digitized on a PowerMac G3 (Apple) computer using Adobe Photoshop version 4.01, and quantified using the UN-SCAN-IT Gel program version 4.3 (Silk Scientific; Orem, Utah).

### 13.1.6 Immunofuorescence (IFA) and Immunohistochemistry Analysis

Cells seeded onto 22 mm² coverslips were fixed and stained with immunoaffinity-purified (IAP) rabbit polyclonal anti-SSeCKS (Lin et al., 1996, J. Biol. Chem. 271:28,430-28,438) or rabbit anti-Gravin sera (Gelman et al., 2000, Histochem. J. 32:13-16) as described previously (Gelman et al., 1998, Cytoskelet. 41:1-17). Immunocytochemistry was performed as described (Gelman et al., 2000, Histochem. J. 32:13-16). Slides were viewed on an Olympus IX-70 fluorescent microscope and digitized using a Sony Catseye camera connected to a PowerMac G3 computer. Image analysis was performed using Adobe Photoshop 4.01.

### 13.1.7 Tumor and Metastasis Formation in Nude Mice

Six-week-old female nude mice (Taconic Farms; Germantown, N.Y.) were injected s.c. with 10⁵ MLL/vector or MLL/SSeCKS clones. The viability of the cells was >90% as determined by trypan blue exclusion. All mice were fed water containing 100 μg/ml tet plus 5% sucrose until the primary tumors were palpable (2-4 mm), at which point, the tet-water was withdrawn. Mice were sacrificed 3 weeks after injection. The primary tumors were measured and weighed, and the lungs were stained for metastases by injecting India ink (30 ml ink plus 4 drops of 1 M ammonium hydroxide, diluted into 200 ml of dH²O) into the trachea for 10 min at room temperature followed by several washes in PBS. Surface metastases, which exclude the dye, were then counted.

### 13.2. Results

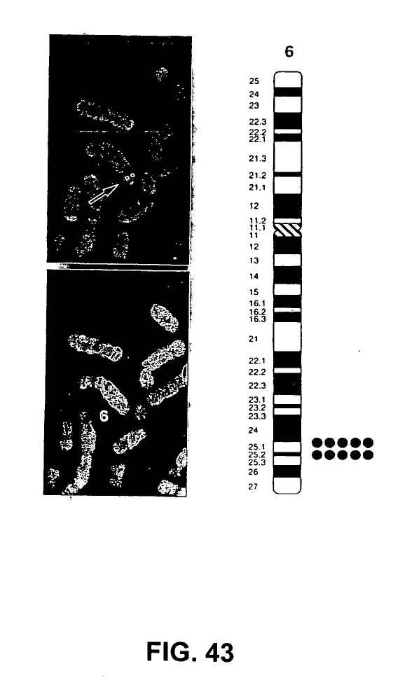

Mapping of SSeCKS, as referred to herein as Gravin. Rodent SSeCKS and human Gravin/AKAP12 show 83% identity over the first ˜1000 a.a., <20% similarity over the next ˜500 a.a., and identity in two 15-a.a. stretches at the C-termini, one of which encodes a PKA anchoring site (Nauert et al., 1997, Curr. Biol. 7:52-62). Full-length SSeCKS cDNA recognizes Gravin mRNA under conditions of stringent hybridization (Gelman et al., 2000, Histochem. J. 32:13-26). Using a Gravin cDNA probe, human gravin was mapped by fluorescence in situ hybridization (FISH) to chromosome 6q24-25.2 (FIG. 43). These map coordinates are confirmed by microsatellite markers (Sanger Sequencing Centre, UK). Secondary hybridization signals were not detected which might reflect a second family member. FISH analysis using a full-length SSeCKS cDNA probe identified the same, singular region. Moreover, mouse SSeCKS maps to the Tsga12 locus at the centromeric end of chromosome 10p, which is syntenic with human chromosome 6q24-27⁴ (Mouse Genome Informatics Web Site; http://www.informatics.jax.org/), strongly suggesting that SSeCKS and Gravin/AKAP12 are orthologues. Deletions in this region are associated with advanced, non-organ confined prostate cancer cases (Isaacs, et al., 1994, Quant. Biol. 59:653-659; Nupponen et al., 1998, Cancer Genet. Cytogenet. 101:53-57; Alers et al., 2000, Lab. Investig. 80:931-942; Crundwell et al., 1996, Int. J. Cancer 69:295-300; Bookstein, et al., 1997, Br. J. Urol. 79(Suppl 1):28-36; Srikantan et al., 1999, Int. J. Cancer 84:331-335; Visakorpi, T., 1999, Ann. Chir. Gynaecol. 88:11-16; Cunningham et al., 1996, Cancer Res. 56:4475-4482; Cooney et al., 1996, Cancer Res. 56:4150-4153; Visakorpi et al., 1995, Cancer Res. 55:342-347), indicating a possible role for SSeCKS/Gravin in prostate oncogenesis.

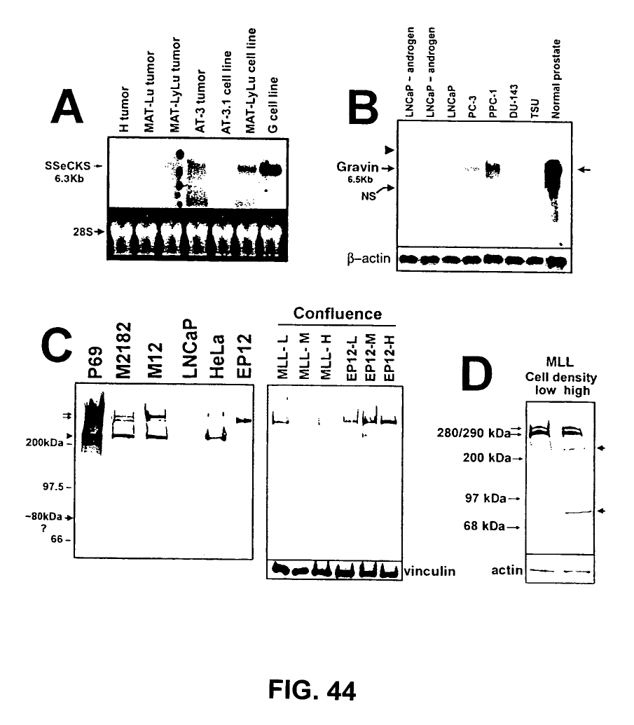

Northern blots containing total or poly (A)-selected RNA from Dunning rat prostate cancer cell lines or tumors (grown in Copenhagen rats) or from human prostate cancer cell lines were probed under stringent conditions with rat SSeCKS cDNA. FIG. 44A shows a progressive loss of SSeCKS RNA signal in MLL and AT-3.1 cancer cells (5- and >20-fold lower, respectively, as defined by densitometry) compared to the mildly transformed G cell line. AT-3.1 cells, which are androgen-independent, are slightly more metastatic in nude mice than MLL (Isaacs, et al., 1986, Prostate 9:261-281). SSeCKS RNA levels in R3327-H-, MatLu-, MLL- or AT-3-induced tumors are reduced >5-fold compared to levels in the G cell line. FIG. 44B shows a similar reduction in Gravin/AKAP12 RNA levels in various human prostate cancer cell lines compared to normal human prostate. In addition to the expected 6.5 Kb gravin message, PC-3 and PPC-1 cells displayed an additional RNA transcript (˜8.5 Kb) that hybridized to the rat SSeCKS probe. Growth of the androgen-responsive LNCaP cells (Schuurmans et al., 1988, Prostate 12:55-63) in the absence of androgens for >9 months had no effect Gravin/AKAP12 transcript levels, possibly because LNCaP cells contain a 6q24-ter deletion (Hyytinen et al., 1997, Br. J. Cancer 75:190-195), a region encompassing gravin (FIG. 43). Thus, the loss of SSeCKS/Gravin message is typical in prostate cancer lines.

The relative levels of SSeCKS/Gravin proteins in various untransformed and cancerous prostate cell lines were compared. In most fibroblastic and epithelial cells, SSeCKS was shown to be expressed as several isoforms 290 kDa (myristylated), 280 kDa (non-myristylated; contains a novel N-terminal 7 a.a. resulting from alternative splicing), 240 kDa (a proteolytic fragment of 290/280 lacking an N-terminal domain), and 43 kDa (an internal proteolytic fragment). The typical Gravin/AKAP12 isoforms are 302,287, 250 and 43 kDa, where the larger sizes of the former three resulting from an extra C-terminal 100 a.a. in Gravin/AKAP12 compared to rodent SSeCKS. These isoforms were confirmed by immunoblotting using several sets of polyclonal and monoclonal antibodies, as well as by mapping with genetic mutants (Gelman et al., 2000, Histochem. J. 32:13-26).

The P69 series (24) consists of human prostate epithelial lines with increasing oncogenic characteristics in nude mice: P69 are non-tumorigenic cells immortalized with SV40 T_{ag}; M2182 are non-metastatic variants that form tumors at the primary injection site; M12 are variants selected in nude mice that form lung metastases following intraperitoneal or intraprostatic injection. FIG. 44C shows a severe decrease (>10-fold) in the levels of the Mr 305/287 kD major Gravin isoforms in both M2182 and M12 cells compared to the parental P69 cells.

Previous data had indicated that the level of SSeCKS RNA and protein increases dramatically under contact-inhibited growth conditions (Lin et al., 1995, Mol. Cell. Biol. 15:2754-2762; Lin et al., 2000, Mol. Cell. Biol. 20:7259-7272; Lin et al., 1996, J. Biol. Chem. 271:28,430-28,438; Nelson and Gelman, 1997, Mol. Cell. Biochem. 175:233-241). FIG. 44C shows a >10-fold increase in SSeCKS protein levels in rat EP12 cultures in transition from subconfluence (L) to several days of confluent growth (H), as determined by densitometry. In contrast, SSeCKS levels in MLL cells, which are 4-fold lower than EP12 cells, are not affected by culture density. However, the abundance of the 240 kD SSeCKS isoform as well as a novel ˜80 kD isoform increases in MLL following growth at high density (FIG. 44D). The 240 kD isoform (250 kD in human cells) is a proteolytic cleavage product lacking NH₂-terminal sequences (Gelman et al., 2000, Histochem. J. 32:13-26). Thus, SSeCKS/Gravin protein levels are induced in both epithelial cells and fibroblasts in response to increased culture density, and this induction mechanism is inactive in prostate cancer cells.

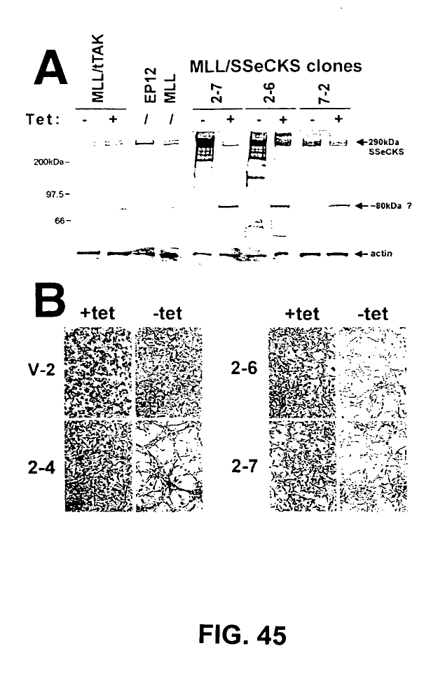

Experiments were conducted indicating that tet-regulated SSeCKS re-expression suppresses MLL-induced oncogenesis. It had been demonstrated previously that the tet-regulated re-expression of SSeCKS led to the suppression of src-induced oncogenic growth fibroblasts (Lin and Gelman, 1997, Cancere Res. 57:2304-2312). In that study, re-expression of SSeCKS to levels 2- to 25-fold over those in untransformed parental cells led to the selective loss of src-induced oncogenic growth parameters such as anchorage- and growth-factor independence and Matrigel invasiveness, but did not inhibit cell proliferation, indicating that the SSeCKS effects were not simply due to toxicity. Because MLL cells encode at least one activated Ha-ras allele (Cooke et al., 1988, Prostate 13:273-287), it was determined whether SSeCKS could suppress ras-associated tumorigenic growth by producing MLL cells with tet-regulated SSeCKS expression. FIG. 45A shows similar levels of SSeCKS protein in the MLL/tTAK cells compared to the parental MLL cells. The MLL/tTAK cells express a tet-regulated form of the tTA transactivator (Shockett et al., 1995, Proc. Natl. Acad. Sci., USA 92:6522-6526) which was found to be much less toxic than the constitutively-expressed tTA. Expression of the 290 kD SSeCKS isoform was induced 4-20 fold in several independently derived clones grown without tet (FIG. 45A). These clones also express background levels of SSeCKS in the presence of tet.

The MLL[tet/SSeCKS] clones exhibited increased cell flattening, decreased refractility, and increased cell-cell interaction following the removal of tet (FIG. 45B). SSeCKS induced a fibroblast-like morphology (FIG. 45B) rather than the epithelial morphology typified by EP12 cells (FIG. 45B, panel c). tTAK caused mild cell flattening (FIG. 45B), and although this was always significantly less than that induced by SSeCKS, it is impossible to exclude that SSeCKS and tTAK work cooperatively in this regard. In contrast, increased cell-cell adhesion was induced by SSeCKS re-expression and not by tTAK.

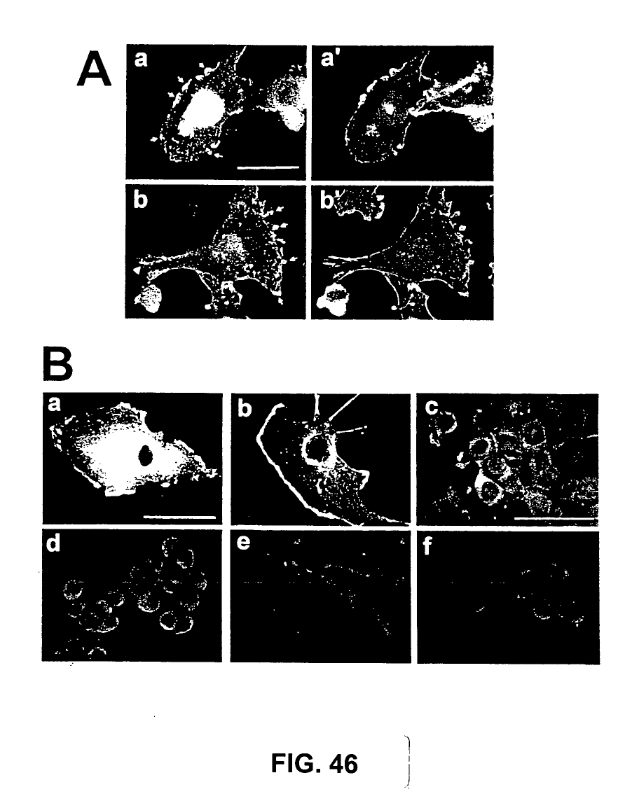

The compartmentalization of SSeCKS was analysed by IFA using immunoaffinity-purified anti-SSeCKS Ig (Lin et al., 1996, J. Biol. Chem. 271:28,430-28,438). FIG. 46 shows enrichment of SSeCKS in the perinuclear regions of EP12 cells as well as a cortical cytoskeletal distribution. SSeCKS was enriched in actin-dense membrane ruffles (FIG. 46A, panels a/a′; arrows), in focal complexes connected to actin stress fibers (FIG. 46A, panels b/b′; triangle) and in actin-dense bundles at the lagging edge (FIG. 46A, panels b/b′; arrows). In MLL[tet/SSeCKS] cells grown without tet, re-expressed SSeCKS was enriched in membrane ruffles (FIG. 46B, panel a) and in lamellipodia (FIG. 46B, panel b).

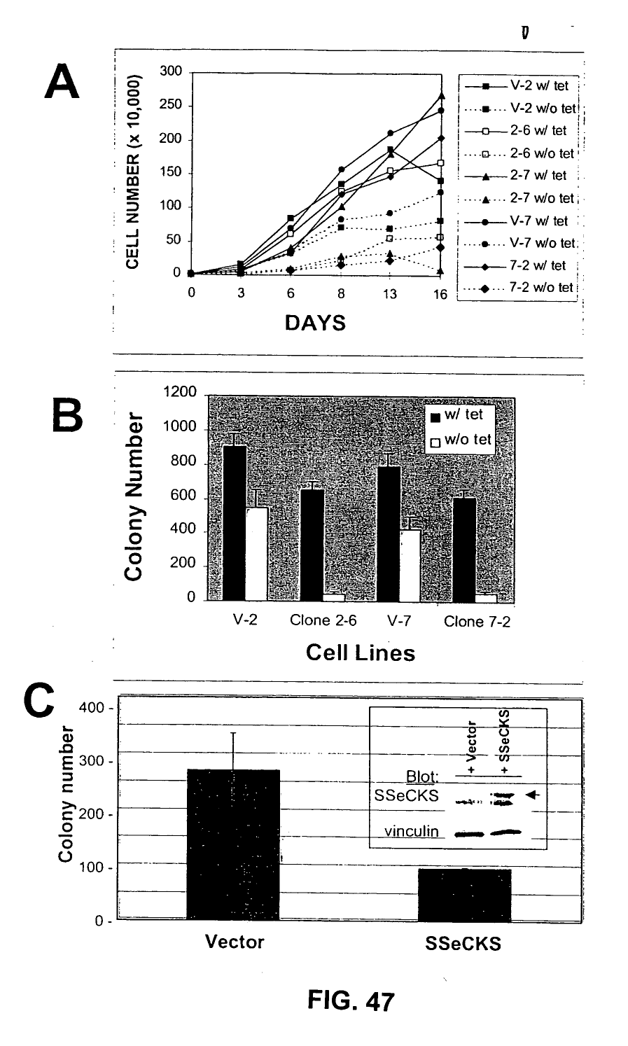

It was next determined whether SSeCKS expression affected parameters of in vitro oncogenic growth. FIG. 47A shows that expression of tTAK alone (V-2 or V-7) was somewhat inhibitory to proliferation in media with 10% serum, probably due to squelching of transcription factors by the VP16 moiety of tTAK (Lin and Gelman, 1997, Cancer Res. 57:2304-2312; Gelman et al., 1998, Cytoskelet. 41:1-17; Gossen et al., 1993, Trends Biochem. Sci. 18:471-475). Although SSeCKS expression in some clones decreased proliferation rates slightly more than vector controls, it is impossible to separate SSeCKS- and tTAK-induced effects. However, FACS-sorted MLL cells transiently co-transfected with SSeCKS and Green Fluorescent Protein (GFP) expression vectors showed no significant changes in proliferation rates compared to GFP controls. Thus, it was concluded that SSeCKS re-expression does not significantly alter the proliferation rate of MLL cells.

FIG. 47B shows that SSeCKS expression inhibited anchorage-independent growth 4-5 fold over that induced by tTAK alone. The roughly 40% decrease in colony forming activity in vector control cells probably relates to the inhibitory effects of tTAK. It was determined whether SSeCKS could inhibit the anchorage-independent growth of MLL in the absence of the tet system. FIG. 47B shows that transient expression of SSeCKS decreased the colony forming efficiency of MLL roughly 3-fold compared to vector controls. Thus, the suppressive effects of SSeCKS on anchorage-independent growth are separable from those of tTAK.

The next set of experiments indicated that SSeCKS/Gravin ˜80 kDa isoform is a marker for prostate cancer. It was noted that the ˜80 kD isoform was present in the MLL/tTAK cells and in all the MLL[tet/SSeCKS] clones grown with tet, whereas tet removal correlated with a 2- to 10-fold decrease in the abundance of this isoform. This isoform was also detected in the human tumorigenic lines M2182 and M12 but not in untransformed parental P69 prostate epithelial cells (FIG. 44C; arrow). It was investigated whether the ˜80 kD isoform was produced by a factor present in either prostate cancer or untransformed cells. NP-40 lysates from MLL and EP12 cells were mixed at a 1:1 ratio to determine if factors from one lysate would decrease the presence of the ˜80 kD isoform. Table 4 (see attached) shows that mixing of the lysates at 30° C. for 5 min caused a decrease in the abundance of the ˜80 kD isoform (compared to incubation of the MLL lysate alone). Pre-boiling of the MLL lysate did not effect the EP12-induced decrease, whereas boiling of the EP12 lysate did. This indicates that EP12 encodes a heat-labile factor that is antagonistic to the production or stability of the ˜80 kD isoform.

TABLE 4
Effect of lysate mixing on the generation
of the Mᵣ 80,000 SSeCKS isoform

| | Presence of Mᵣ | Length of | |
|---|---|---|---|
| Reactants^{a} | 80,000 band | incubation | |
| MLL lysate + EP12 lysate | − | 5 | min |
| MLL lysate (boiled) + EP12 lysate | − | 5 | min |
| MLL lysate + EP12 lysate (boiled) | + | >30 | min |

^{a}MLL and EP12 lysates were added at a 1:1 ratio.

Experiments were conducted to determine whether SSeCKS expression could inhibit either growth of primary tumors or generation of secondary lung metastases. Nude mice were injected in their flanks with 10⁵ cells and then maintained on tet in their drinking water until tumors were palpable (2-4 mm), whereupon the tet-water was removed.

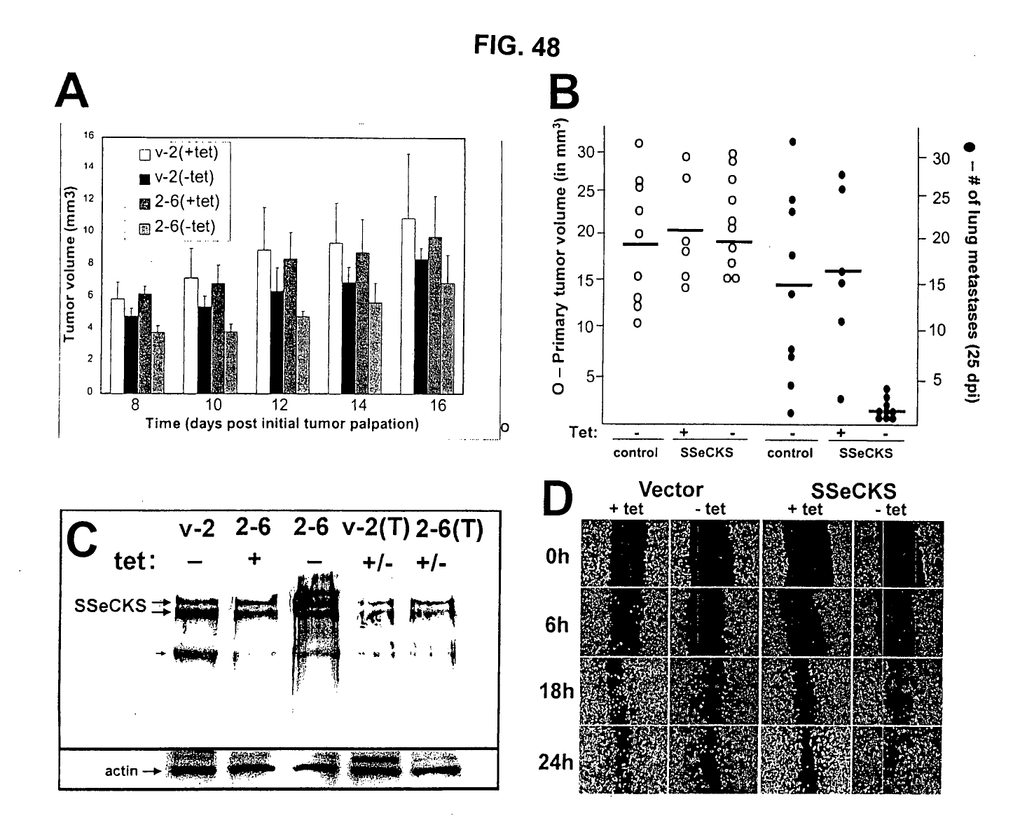

Several independently-derived vector control and SSeCKS re-expressor clones were compared in this analysis to increase significance. SSeCKS expression only mildly inhibited tumor growth at the primary site in comparison to vector controls. The small effect (20% reduction) was 8-10 days after initial tumor palpation, but subsequently, there was no significant difference in tumor size or doubling rates between SSeCKS and control tumors (FIG. 48A). However, analysis of progressing primary-site MLL[tet/SSeCKS] tumors revealed a loss of tet-regulated SSeCKS expression (FIG. 48C), suggesting that the progressors were variants that had defeated the inducible expression of SSeCKS. Growth of cells in vitro from these primary tumors (in DME+hygromycin/puromycin) showed a lack of inducible SSeCKS in the absence of tet. These data indicate that SSeCKS re-expression did not significantly decrease the formation or growth rate of primary-site tumors.

In contrast, mice receiving the MLL[tet/SSeCKS] cells in the absence of tet contained far fewer lung metastases three weeks after primary tumor cell injection than the vector controls (Table 5, FIG. 48B). The inhibition occurred with two independent clones with varying degrees of inducible SSeCKS expression (2-6 and 7-2). Because expression of SSeCKS had no effect on cell motility in a monolayer wounding assay (FIG. 48D), it is unlikely that these cells are defective for cell motility in vivo. These data indicate that even mild re-expression levels of SSeCKS, as in clone 7-2, are sufficient to suppress metastatic potential in MLL cells.

TABLE 5
Effect of ectopic SSeCKS expression on lung metastasis formation

| Clone injected^{a} | No. of lung metastases^{b} | Size of metastases^{c} |
|---|---|---|
| V-2 | 32, 6, 3 | 4 mm ± 1 |
| V-7 | 18, 11, 6 | 4 mm ± 1 |
| 2-6-11^{d} | 0, 1, 0 | 1 mm ± 0.5 |
| 7-8-14^{d} | 0, 0, 0 | 0 |
| 2-6-5^{d} | 2, 0, 0 | 1 mm ± 0.5 |

^{a}10⁵ cells (>90% viability) injected s.c. into flanks.
^{b}Lung metastases were analyzed 3 wks after initial appearance of primary tumors.
^{c}Metastases on the surface of lungs were identified by exclusion of India ink staining as described in “Materials and Methods.”
^{d}Subclones of MLL[tet/SSeCKS] lines 2-6 and 7-8 isolated by single-cell cloning methods were identified, which had >95% of cells overexpressing SSeCKS (−tet) using IFA analysis (data not shown).

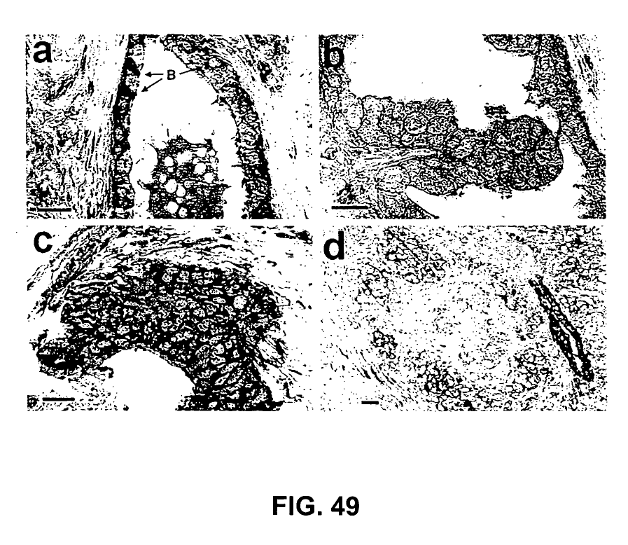

Experiments further demonstrated loss of SSeCKS/Gravin expression in well-differentiated human prostate cancer. Various human prostate lesions were analysed for SSeCKS/Gravin expression. SSeCKS/Gravin stained extensively in prostatic epithelial cells, especially the basal epithelial cells, although cell surface staining was detected in some columnar epithelial cells (FIG. 49, panel a). Abundant SSeCKS/Gravin staining was detected in benign prostatic hyperplastic lesions (FIG. 49, panel b) and in well-differentiated carcinomas (FIG. 49, panel c). In contrast, SSeCKS/Gravin staining was absent in undifferentiated carcinomas (FIG. 49, panel d). Non-cancerous ducts in the same prostate contain epithelia with robust SSeCKS/Gravin staining. A larger survey of human samples showed a consistent loss of SSeCKS/Gravin expression in MPA with Gleason sums >6 (Table 6). MPAs were all positive for prostatic acid phosphatase as a control for stainability. These data correlate the loss of SSeCKS/Gravin with the onset of aggressive prostate oncogenesis in humans.

TABLE 6
Loss of Gravin expression in malignant prostate cancer

| Tissue | Gleason sum | SSeCKS/Gravin | PAP^{a} |
|---|---|---|---|
| normal prostate − 1 | | +++ | + |
| normal prostate + 2 | | +++ | + |
| BPH − 1 | | +++ | + |
| BPH − 2 | | ++ | + |
| BPH − 3 | | +++ | + |
| BPH − 4 | | +++ | + |
| BPH − 5 | | +++ | + |
| BPH − 6 | | ++ | + |
| BPH − 7 | | +++ | + |
| BPH − 8 | | ++ | + |
| BPH − 9 | | +++ | + |
| BPH − 10 | | +++ | + |
| CaP − 1 | 4 | ++ | + |
| CaP − 2 | 4 | ++ | + |
| CaP − 3 | 5 | +/−^{b} | + |
| CaP − 4 | 6 | +/− | + |
| CaP − 5 | 6 | +/_{—} | + |
| CaP − 6 | 7 | − | + |
| CaP − 7 | 9 | − | + |
| CaP − 8 | 9 | − | + |
| CaP − 9 | 9 | − | + |
| CaP − 10 | 9 | − | + |
| CaP − 11 | 10 | − | + |

^{a}PAP, Prostatic acid phosphatase.
^{b}Staining in neoplastic regions; normal ducts in the same samples displayed typical epithelial cell staining.

### 14. EXAMPLE

### SSeCKS Regulates Progression Through the Cell Cycle by Controlling Cyclin D Activity

The data presented below indicates that SSeCKS controls progression through the cell cycle by regulating the expression and localization of cyclin D.

### 14.1. Materials and Methods

### 14.1.1. Cells

S2-6 cells (gift of David Schatz, Yale School of Medicine), NIH3T3 cells expressing a tetracycline (tet)-regulated version of the tet transactivator, tTA (Shockette et al., 1995, Proc. Natl. Acad. Sci. USA 92:6522-6526) were grown in histidine-deficient Dulbecco's modified Eagle's media (DMEM; Irvine Scientific) supplemented with 0.5 μM L-histidinol (Sigma; St. Louis, Mo.), 10% calf serum, penicillin/streptomycin/amphotericin B (GIBCO; Gaithersburg, Md.), and 0.5 μg/ml of tetracycline (Sigma). NX cells, an ecotropic packaging line (a gift of Gary Nolan, Stanford University), were grown in DMEM media supplemented with 10% calf serum.

### 14.1.2 Tetracycline-Regulated SSeCKS Overexpressing Cell Lines

An Eco RI fragment encoding the full-length SSeCKS cDNA was spliced into pUHD10-3, a plasmid containing a tTA-dependent promoter (gift of Hermann Bujard (Gossen and Bujard, 1993, Trends Biochem. Sci. 18:471-475)). 10 μg of pUHD10-3/SSeCKS or pUHD10-3 DNA were co-transfected into S2-6 cells with 1 μg of pBabepuro using CaPO4 precipitates. Stable cell lines were selected in S2-6 media supplemented with 2 μg/ml of puromycin (Sigma) and 5 μg/ml tet. S2-6/S24, an SSeCKS clone, and S2-6/V3, transfected with vector alone, were used to produce cyclin D1 overexpressors.

### 14.1.3. Cyclin D1 Overexpressing Cell Lines

Stocks of ecotropic viruses encoding pLJ/cyclin D1 (gift of Robert Krauss, Mount Sinai School of Medicine) or pLJ retrovirus vector were produced by transient transfection of φNX cells (24) and then filtering media through 0.2 μm low protein-binding filters (Gelman Sciences). Stably infected cell lines were selected in S2-6 media supplemented with 400 μg/ml of G418 (GIBCO) and 0.5 μg/ml of tet.

### 14.1.4. Western Blot Analysis

Cells were washed thrice with ice-cold PBS (phosphate buffered saline), scraped into microtubes, and lysed with RIPA buffer (10 mM Tris-HCl pH 7.4, 150 mM NaCl, 5 mM EDTA, 8% glycerol, 1% Triton X-100, 0.1% SDS, 0.5% sodium deoxycholate, 1 mM sodium vanadate, 10 mM sodium fluoride, 1 mM phenylmethylsulfonyl fluoride (PMSF), 2 μg/ml of aprotinin, 2 μg/ml of leupeptin, 2 μg/ml of antipain, 2 μg/ml of pepstatin). Protein content was normalized using Bio-Rad Protein Assay Kits (Bio-Rad Laboratories). Equal amounts of protein were separated by SDS-PAGE (5%), electrophoretically transferred to PolyScreen PVDF membrane (NEN; Boston, Mass.) and immunoblotted as described before (Gelman et al., 1998, Cell Motil. Cytoskeleton 41:1-17). Primary polyclonal (PAb) and monoclonal (MAb) antibodies included: SSeCKS (PAb; ref. 40), ERK2 (PAb), cyclin D1 (PAb), cyclin A or E (MAb), CDK2, 4 or 6 (MAb), CKIs p21 and p27 (MAb; Santa Cruz Biotechnology), CKIs p18 and p19 (MAb; gifts of Selina Chen-Kiang, Weill Medical School of Cornell University), pRb or cyclin D1 (MAb; PharMingen), or CKI p16 (MAb; Clontech). Following three PBS washes, the filter was incubated with either horseradish peroxidase- (Chemicon) or alkaline phosphatase- (Boehringer Mannheim) conjugated secondary antibody for 1 h. After extensive washing, the secondary antibodies were visualized using ECL (Amersham) or Western Blue (Promega) substrates, respectively. For detection of pRb phosphorylation, cells were lysed in NETN buffer (1% NP-40, 2 mM EDTA, 50 mM Tris-HCl pH8.0, 250 mM NaCl, 1 mM dithiotreitol, 1 mM Na3VO4, 10 mM NaF, 1 mM PMSF, 2 μg/ml of aprotinin, 2 μg/ml of leupeptin, 2 g/ml of antipain, 2 g/ml of pepstatin) followed by SDS-PAGE and immunoblotting. In some cases, blots were stripped of antibody probes by incubating in 500 ml of pre-heated (50° C.) 62.7 mM Tris-HCl, pH 6.7, containing 2% SDS and 0.1 M β-mercaptoethanol, followed by extensive washes in PBS.

### 14.1.5. Proliferation Assay

10⁴ cells were seeded onto 24-well plates and the next day an aliquot of cells was trypsinized and counted to establish a baseline plating efficiency. The remaining cells were grown in media in the presence or absence of tetracycline. Duplicate wells were trypsinized and counted every two days using a hemacytometer (Fisher Scientific).

### 14.1.5. ERK2 Kinase Assay

Cells were serum-starved overnight and then stimulated with 10% calf serum-containing media for various periods. Following lysis in RIPA buffer, the lysates were incubated with rabbit anti-ERK2 antibody prebound to Affi-Prep protein A beads (BioRad). The immunocomplex was washed twice with RIPA buffer and twice with kinase buffer (10 mM Hepes pH 7.5, 10 mM magnesium acetate). 20 μl of the bead-antibody-antigen complex was resuspended in 40 μl containing a 1:1 dilution of myelin basic protein (MBP, 2 mg/ml; Sigma) and 3× hot mix (30 mM HEPES pH7.5, 30 mM magnesium acetate, 150 μM ATP, 10 μCi γ-³²P[ATP]), and incubated for 30 min at 30° C. The reaction was stopped by adding 60 μl of 2× protein loading dye. This mixture was boiled and electrophoresed through a 15% SDS-polyacrylamide gel, followed by autoradiography.

### 14.1.6. CDK2 Kinase Assay

RIPA lysates were incubated with anti-cyclin E antibodies prebound to protein A beads. The immunocomplexes were washed three times with RIPA buffer and two times with histone Hi assay buffer (50 mM HEPES pH7.5, 150 mM NaCl, 10 mM MgCl₂, 2 mM EGTA, 1 mM DTT), and then resuspended in 25 μl of assay buffer supplemented with 20 M ATP and 4 μg Histone H1 (GIBCO). The kinase assay was initiated by adding 10 μCi of γ-³²P[ATP]. After 10 min of incubation at 30° C., the supernatants were collected and electrophoresed through a 10% SDS-polyacrylamide gel, followed by autoradiography.

### 14.1.7. Cell Cycle Analysis

The percentage of cells in different phases of cell cycle was quantified using flow cytometry as described (Zhu et al., 1993, Genes Dev. 7:1111-1125; Kaplan et al., 1998, Mol. Cell. Biol. 18:1996-2003). Synchronized cells were harvested by trypsinization, washed in PBS, and fixed in ice-cold 70% ethanol (10⁶ cells/ml) for at least 2 h at −20° C. Before flow cytometric analysis, the pelleted cells were washed in PBS and stained for 2 h at room temperature with 20 μg/ml propidium iodide (Sigma) containing 1 μg/ml of RNase A. Analysis was performed on a FACScan machine (Becton Dickinson) using the CellFIT analysis software.

### 14.1.8. Construction and Expression of GST-SSeCKS-CY Motif Fusion Proteins

SSeCKS2 fragments (a.a. residues 389-552) were generated by PCR amplification and cloned in pBluescript II KS (Stratagene)(40). Mutations in two potential CY motifs were generated using a Transformer™ Site-Directed Mutagenesis Kit (Clontech). A unique restriction site in pBluescript, Sca I, was chosen as a selection marker (Sca I to Stu I). “Trans” and “switch” selection primers were: 5′GTGACTGGTGAGGCCTCAACCAAGTC (Sca I to Stu I) and 5′GTGACT GGTGAGTACTCAACCAAGTC (Stu I to Sca I), respectively. Trans-mutagenic primers were as follows: 5′GGAAGTCCCTTGTCGAGCCTCTTCAGTAGC (first KK to SS), 5′GCTC AGGCTTAAGCTCGCTGTCTGGG (second KK to SS), 5′CCCTTGAAGAAAAGC TTCAGTAGC (first L to S), 5′GGCTTAAAGAAGTCGTCTGGGAAG (second L to S). Switch-mutagenic primers were: 5′CCCTTGTCGAGCAGCTTCAGTAGC (first L to S) and 5′GGCTTAAGCTCGTCGTCTGGGAAG (second L to S). After denaturation, the target SSeCKS2 plasmid was annealed with primers, followed by synthesis of the mutant strand DNA. Primary selection was carried out by restriction digestion. The mutated plasmid was amplified, and then was subjected to a second round of restriction enzyme digestion. All mutations were confirmed by sequencing using Sequenase 2.0 kits (US Biochemicals). The resulting SSeCKS2 variants were spliced back to pGEX 5x-1 for fusion protein expression. BL21 (DE3) pLysS bacteria (Novagen) were transformed with these constructs, grown in LB/Amp medium containing 20 mM glucose at 37° C., and GST-fusion protein induced and purified as described previously (Lin et al., 1996, J. Biol. Chem. 271:28430-28438; Sambrook et al., 1989, Molecular cloning: a laboratory Manual, Cold Spring Harbor Laboratory Press).

### 14.1.9. In vitro Cyclin Pull Down Assay

Cyclin D1 pull-down assays were performed as described previously (Chen et al., 1996, Cancer Res. 56:3168-3172 (Erratum, 56:4074)). Briefly, cells were lysed in binding buffer (20 mM Tris-HCl pH7.4, 1 mM EDTA, 25 mM NaCl, 10% glycerol, 0.01% Nonidet P-40, 1 mM each of DTT, Na3VO4 and PMSF, 2 μg/ml each of aprotinin, leupeptin, antipain, pepstatin A). 500 μg of the lysates were incubated with 15 μg of GST-SSeCKS2 or GST prebound to glutathione-Sepharose beads for 3 h at 40° C. on a rotating wheel. After 4 washes in binding buffer, the beads were boiled in protein loading dye, and the proteins analyzed by immunoblotting using anti-cyclin D1 antibody.

### 14.1.10. Immunofluorescence Analysis

Cells grown on 22 mm² coverslips were fixed in 60% acetone/2% formaldehyde at −20° C. for 20 min and then incubated with either immunoaffinity-purified anti-SSeCKS (40) and/or anti-cyclin D1, and then stained with FITC- or TRITC-labeled secondary antibodies as described previously (Gelman et al., 1998, Cell Motil. Cytoskeleton 41:1-17). Slides were visualized on either a Leica CLSM laser confocal microscope or an Olympus IX-70 fluorescent microscope fitted with a Sony Catseye digital camera, and digital images were processed using Photoshop 4.01 and NIH Image software on a Macintosh Power PC 8100/100 AV.

### 14.1.11. Peptide Treatment

Peptides were synthesized by BioWorld 2000 or the Mount Sinai Peptide Core Facility and were >85% pure as determined by ion-spray mass spectroscopy. The following peptides were produced, either linked to penetratin peptide (RQIKIWFQNRRMKWKK) or as the sequences shown: wt SSeCKS CY (LKKLFSSSGLKKLSGK), mutated CY (LSSSFSSSGLSSSSGK) or phosphoserine CY (LKKLFS^{Pi}SSGLKKLSPiGK). N-terminal biotinylation was performed on half the penetratin and half the non-penetratin-linked peptide product. Peptides were resuspended in DMEM and then incubated with cells at a final concentration of 100 μg/ml for 2-4 h. Peptide entry into cells was monitored by fixation of cells in ice cold ethanol/acetone (9:1) for 5 min at −20° C., washing with DMEM/10% CS, and incubation with PAb anti-cyclin D1, then with TRITC-labeled anti-rabbit Ig (Chemicon) and FITC-labeled avidin (Molecular Probes). Coverslips were mounted and photographed as described above.

### 14.1.12. Cell Fractionation

Cytosolic and nuclear fractions were prepared according to the technique of Hochholdinger et al., 1999, Mol. Cell. Biol. 19:8052-8065, with the following modifications. After centrifuging the homogenate at 500 g for 10 min at 4° C., the supernatant (typically 1 ml) was collected and SDS and Triton X-100 were added to final concentrations of 0.2% and 1%, respectively, followed by vortexing and storage at −70° C. The pellets (nuclear fractions) were resuspended in 100-200 μl of “hypotonic lysis buffer” (1 mM EDTA, 1 mM EGTA, 10 mM β-glycerophosphate, 1 mM Na₃VO₄, 2 mM MgCl₂, 10 mM KCl, 1 mM dithiothreitol, 40 μg/ml PMSF, 10 μg/ml aprotinin and 10 μg/ml leupeptin). This fraction was loaded atop 1 ml of 1 M sucrose in hypotonic lysis buffer and centrifuged at 1600 g for 20 min at 4° C. The pellets were resuspended in 100 μl of buffer, brought to final concentrations of 0.2% SDS and 1% Triton X-100, vortexed and stored at −70° C.

### 14.2. Results

Previous attempts to produce stable constitutive expression of SSeCKS resulted in the selection of variants deleted of their transduced SSeCKS cDNA copies (39). Using S6 cells, cells lines were produced which express full-length rat SSeCKS following the removal of tet (Gelman et al., 1998, Cell Motil. Cytoskeleton 41:1-17). A number of resulting cell lines, e.g.—S24 and S33, showed background levels of SSeCKS in the presence of tet and >25-fold induction of the 290 kDa SSeCKS isoform following tet removal (FIG. 50A). At least 40% of the cells lines selected showed a similar profile of inducibility of apparently full-length protein. As described previously (Gelman et al., 1998, Cell Motil. Cytoskeleton 41:1-17), overexpression of SSeCKS caused severe cell flattening and the production of exaggerated cell projections (FIG. 50B).

The effect of SSeCKS overexpression on proliferation rates in the presence of serum growth factors was tested. In vector control cells, removal of tet decreased proliferation rates 20-40% (FIG. 50C). This phenomenon is most likely due to squelching of endogenous transcription factors by the VP-16 moiety of the tTA. In contrast, S24 cells expressing full-length SSeCKS growth arrested following tet removal, showing only a marginal increase in cell number after 10 days of incubation in the presence of serum growth factors (FIG. 50C). The finding of SSeCKS-induced growth arrest in a number of independently-derived clones (Table 7) indicates that this phenomenon is not due to the idiosyncrasies of a particular cell line.

TABLE 7
Percent of control (V) or SSeCKS (S)
cells in various cell cycle phases

| % of cells in phase^{b}: | | |
|---|---|---|
| G_{O}/G₁ | S | G₂/M |

| Clone | +TET | −TET | +TET | −TET | +TET | −TET |
|---|---|---|---|---|---|---|
| V3 | 54.6 | 58.9 | 20.0 | 19.2 | 25.2 | 21.9 |
| V9 | 59.9 | 63.2 | 23.2 | 23.0 | 16.9 | 13.8 |
| S24 | 54.5 | 72.5 | 23.9 | 11.7 | 21.6 | 15.8 |
| S26 | 49.6 | 70.2 | 26.9 | 14.2 | 23.5 | 15.5 |
| S33 | 54.3 | 72.1 | 23.9 | 10.2 | 21.8 | 17.7 |
| S23^{a} | 60.8 | 63.7 | 21.1 | 18.4 | 18.1 | 17.9 |
| S38^{a} | 60.9 | 56.3 | 21.7 | 21.8 | 17.4 | 21.8 |

^{a}Cell lines expressing slightly truncated SSeCKS product.
^{b}Cells were grown and labeled with propidium iodide as described in Materials and Methods. Percentages are based on a single experiment in which all clones were grown under the same condition (with or without TET [+TET and −TET, respectively]). Two repeats of the experiment showed less than a 10% variation for individual clones

FIG. 50D shows that unlike p53 overexpression, which induces cell senescence (Sugrue et al., 1997, Proc. Natl. Acad. Sci. USA 94:9648-9653), re-addition of tet after 6 days resulted in the full recovery of proliferative ability. On the possibility that the proliferating cells represented a “breakout” population, 20 S24 subclones were isolated and shown to recover equally from growth arrest by the re-addition of tet (data not shown). The proliferating cells could be re-arrested by re-adding tet, indicating that these cells were still SSeCKS-responsive. However, after >4 rounds of arrest/release, it became more difficult to induce full arrest, likely due to the selective advantage of proliferating variants.

To determine where in the cell cycle SSeCKS arrests cell proliferation, S24 or control cells were put into G0 phase by serum starvation, then induced with serum in the presence or absence of tet, followed by propidium iodide staining and FACS analysis. Table 8 shows a 2-3 fold reduction in the percentage of S phase following expression of ectopic SSeCKS.

TABLE 8
Correlation between G₁ phase growth arrest,
cell flattening, and loss of cyclin D1

| | Cell Line | | | | | | |
|---|---|---|---|---|---|---|---|
| Observation | V3 | V9 | S24 | S26 | S33 | S23^{a} | S38^{a} |
| Flattening | − | − | + | + | + | − | − |
| Growth arrest | − | − | + | + | + | − | − |
| G₁ arrest | − | − | + | + | + | − | − |
| Suppression of cyclin D1 | − | − | + | + | + | − | − |

^{a}Cell lines expressing slightly truncated SSeCKS product.

Several independently derived tet/SSeCKS clones (S26 and S33) showed similar S phase decreases concomitant with increases in G1 phase (Table 1) indicating an overall G1 phase arrest. Interestingly, a small number of tet/SSeCKS clones (<15% of all clones derived) typified by S23 and S38, showed neither G1 arrest nor cell flattening (Table 2). Although these clones express apparently full-length protein, a more careful analysis (long-run SDS-PAGE analysis using 5% gels) showed that these clones contain small truncations.

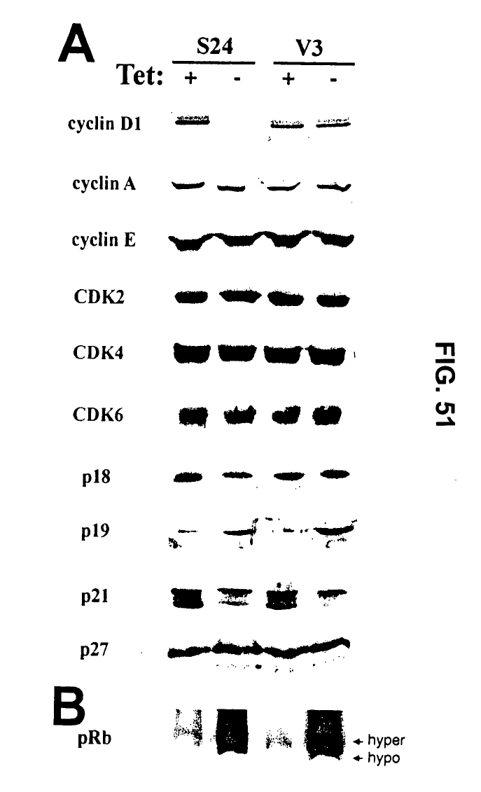

To investigate which cell cycle components are affected by SSeCKS overexpression, cell lysates from S24 and V3 cells were analyzed by western blotting. Among all the components examined, including cyclins D1, E and A, CDKs 2, 4 and 6, and CKIs (p16, p18, p19, p21 and p27), only the expression of cyclin D1 was dramatically reduced in S24 cells grown in the absence of tet in comparison with V3 cells (FIG. 51A). The expression of p16 was undetectable in these NIH3T3-derived cell lines. p21 expression was decreased in both S24 and V3 cells after removal of tet, probably due to the nonspecific effect of tTA. Similarly, the level of p19 was increased in both S24 and V3 cells in the absence of tet. However, the absence of pRb hyperphosphorylation in S24 cells after tet removal (FIG. 51B) correlates with G1 arrest due to cyclin D1 deficiency.

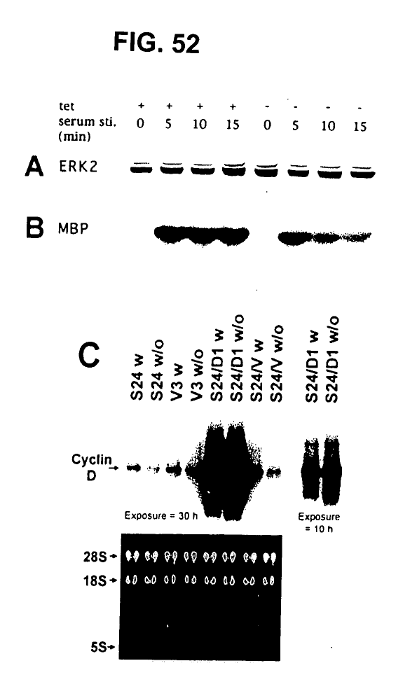

Because the expression of cyclin D1 is dependent on sustained ERK activation (1), it was determined whether SSeCKS affects serum-inducible ERK activation. We previously showed that SSeCKS′ ability to suppress src-induced oncogenesis correlated with a growth factor-independent superinduction of ERK-2 activity (Lin and Gelman, 1997, Cancer Res. 57:2304-2312), indicating that SSeCKS could modulate ERK-activating mechanisms. FIG. 52B shows that serum-inducible ERK2 activation, as measured by the ability of ERK2 immunoprecipitates to phosphorylate myelin basic protein (MBP), was depressed 5-fold by SSeCKS overexpression. Moreover, SSeCKS attenuated the length of ERK2 activation, presumably to levels insufficient for cyclin D induction. SSeCKS expression had no effect on ERK2 protein levels (FIG. 52A). Finally, FIG. 52C shows that tet removal resulted in a loss in steady-state cyclin D message levels in the S24 cells but not in the V3 controls. Thus, SSeCKS may inhibit cyclin D transcription by down-modulating ERK2 activation.

It had been shown previously that SSeCKS overexpression caused dramatic morphological changes including cell flattening, a transient loss of F-actin stress fibers and vinculin-associated adhesion plaques, and the formation of filopodia- and lamellipodia-like projections (Gelman et al., 1998, Cell Motil. Cytoskeleton 41:1-17). Table 7 shows a correlation between these effects and SSeCKS-induced G1 arrest in several independent tet/SSeCKS clones. In contract, the variant clones such as S23 neither flattened nor arrested in G1. Additionally, G1 arrest always correlated with a loss of cyclin D1 whereas the non-arresting clones expressed cyclin D1 in the absence of tet. These data demonstrate a strong correlation between SSeCKS-induced G1 arrest, loss of cyclin D1, and SSeCKS-induced cytoskeletal reorganization.

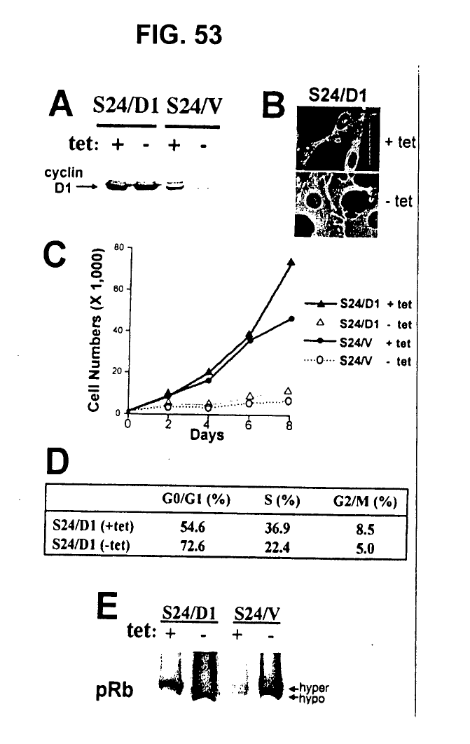

If loss of cyclin D1 was sufficient to induce the G1 arrest, it was believed that the forced expression of exogenous D1 should rescue the SSeCKS-induced arrest. FIG. 53A shows that following retroviral transduction of D1 into S24 cells, D1 levels were unaffected by SSeCKS overexpression. Surprisingly, the forced expression of D1 failed to rescue the G1 arrest as shown by a lack of proliferation (FIG. 53C), by the continued flattened cell morphology of S24/D1 cells grown without tet (FIG. 53B) and by a loss of S phase staining in FACS analysis (FIG. 53D). Most significantly, pRb remains hypophosphorylated in S24/D1 cells grown in the absence of tet (FIG. 53E). However, there was no change relative to S24/V controls in the steady-state levels of cyclins E and A, CDKs 2, 4, and 6, CKIs p15, p18, p19, p21 or p27, or in the total cell CDK4-, 6- or 2-associated kinase activities.

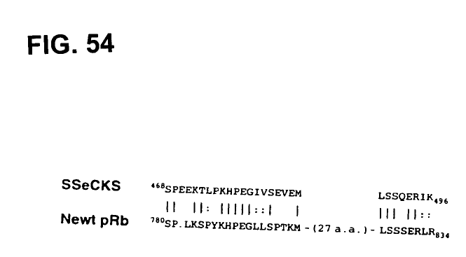

Recent data indicate that binding of c-Abl protein to the C-terminus of pRb is required along with pRb hyperphosphorylation to insure G1→S transition (36). Because SSeCKS (a.a. 468 to 496) shows similarity with the C-terminal domain of pRb involved in c-Abl binding (the Rb “C-pocket”; a.a.-780-860; FIG. 54), it was believed that overexpressed SSeCKS might compete directly for c-Abl binding and thus, inhibit Rb-mediated G1→S transition. However, overexpression of SSeCKS did not affect the levels of pRb-bound c-Abl.

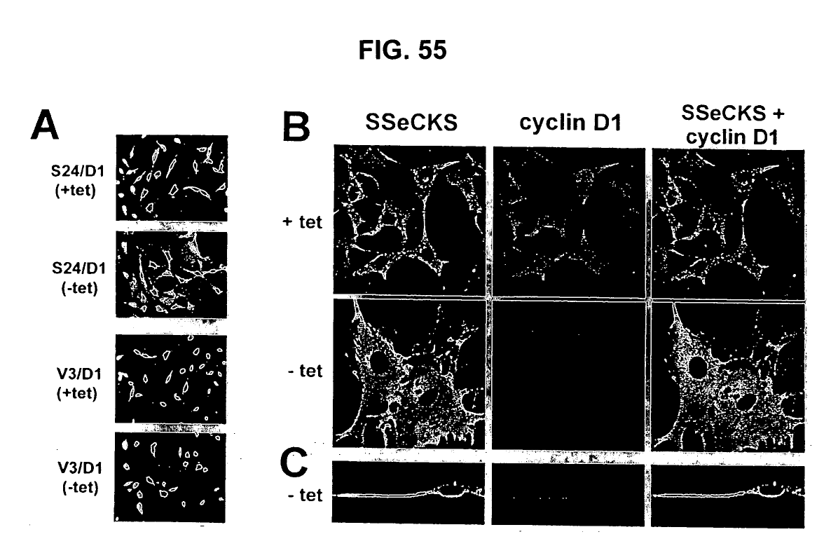

It was possible that active D1/CDK4 complexes were not accessible to their downstream target, pRb. FIG. 55 shows that the majority of cyclin D1 in S24/D1 cells grown without tet is cytoplasmic in contrast to cells grown with tet where most of the staining is nuclear. Additionally, confocal microscopy shows that D1 co-localizes with SSeCKS in the cytoplasm (FIG. 55B). Although some S24/D1 cells grown without tet exhibited nuclear staining, there was a 70% reduction in the amount of nuclear D1 and the number of cells with nuclear staining compared to controls (Table 9). These data indicate that SSeCKS induces the cytoplasmic sequestration of ectopic D1.

TABLE 9
Percentage of cells showing nuclear or
cytoplasmic staining of cyclin D1^{a}

| | | % of cells showing | |
|---|---|---|---|
| | | staining of | |
| Cell type | TET | Cytoplasm | Nucleus |
| V3/D1 | + | 3 ± 3 | 97 ± 4 |
| V3/D1 | − | 3 ± 2 | 93 ± 4 |
| S24/D1 | + | 4 ± 2 | 94 ± 4 |
| S24/D1 | − | 98 ± 4 | 24 ± 5 |
| S24/D1 + PMA^{c} | + | NA^{d} | 95 ± 4 |
| S24/D1 + PMA | | NA | 78 ± 5 |
| 293T + D1-pBABEhygro (1:2.5)^{b} | | 22 ± 6 | 86 ± 4 |
| 293T + D1-SSeCKS (1:2.5) | | 78 ± 4 | 27 ± 4 |
| 293T + D1-pBABEhygro (1:10) | | 35 ± 4 | 80 ± 2 |
| 2293T + D1-SSeCKS (1:10) | | 73 ± 6 | 18 ± 4 |

^{a}Cells grown on 22-mm² coverslips under various TET conditions (+, with; −, without) for 2 days were fixed and stained for cyclin D1 using PAb, as described in Materials and Methods. Independent fields of cells were counted (total of 250 to 300 cells for each analysis).
^{b}293T cells were transfected transiently with Lipofectamine (GIBCO-BRL) containing pEGFP-1, pRcCyclin D1 (gift of A. Dutta) plus pBABEhyg, or pBABEhyg/SSeCKS at either a 1:2.5 or 1:10 ratio, fixed after 40 h, and then stained for cyclin D as described above.
^{c}Cells grown with PMA (200 nM final concentration) for 30 min.
^{d}NA, not applicable because PMA treatment decreased the cytoplasmic, but not nuclear area.

On the possibility that the SSeCKS-induced sequestration of cyclin D was an artifact of a particular cell lines (i.e.—due to the tTa transactivator, for example, 293T cells were transiently transfected with a cyclin D1 expressor plasmid with excess molar ratios of either an SSeCKS expressor plasmid or vector alone. Table 9 shows that SSeCKS induced a 3-4 fold increase in cytoplasmic cyclin D1 compared to vector alone. This clearly shows that SSeCKS can direct the cytoplasmic sequestration of cyclin D1 in several cell types, under conditions of both transient and stable expression, and in the absence of the tet-regulated system

SSeCKS binds G1 phase cyclins in vitro via tandem CY motifs. A so-called cyclin-binding (CY) motif which facilitates the binding of cyclins to several cell cycle components such as p21 (Chen et al., 1996, Mol. Cell. Biol. 16:4673-4682). SSeCKS encodes two closely spaced potential CY motifs, KKLFSxxxxKKLSG (K/RK/R followed by two nonpolar residues, with the first usually Leu). This domain also contains two major in vivo PKC sites, Ser507 and Ser515 (Lin et al., 1996, J. Biol. Chem. 271:28430-28438; Chapline et al., 1996, J. Biol. Chem. 271:6417-6422). It was tested whether a GST fusion protein containing the SSeCKS CY motifs (“SSeCKS-2”) could bind G1 phase cyclins in an in vitro pulldown assay. Indeed, GST-SSeCKS-2, but not GST alone, bound endogenous and ectopic D1 from lysates prepared from S24, S24/D1, S24/V and V3 cells grown in the presence or absence of tet. Stripping of the blot and reprobing with cyclin E-specific antibody showed that GST-SSeCKS2 also bound cellular cyclin E. The levels of cyclins D1 or E bound by GST-SSeCKS2 corresponded to their relative stoichiometry in the cells tested, indicating saturation in the binding kinetics. Thus, higher amounts of D1 were bound in the S24/D1 cell lysates irrespective of tet conditions, whereas in S24 cells, where SSeCKS overexpression suppresses D1 levels, less D1 was bound in the [−]tet condition compared to the [+]tet condition. In contrast, the binding to cyclin E was relatively constant throughout the cells lines, reflecting the similar levels of cyclin E in these cells, whether in [−]tet or [+]tet conditions. Additionally, prephosphorylation of GST-SSeCKS2 with rabbit brain PKC (Upstate Biotechnology) ablated cyclin D and E binding.

Mutation of either the up- or downstream KK residues reduced cyclin D1 binding roughly 70%; KK→SS in both motifs reduced binding >95%. In contrast, the L→S mutations had little effect on D1 binding. Importantly, none of the mutations affected the expression level or stability of the bacterially-expressed GST-SSeCKS fusion products. These data show a dependence on the charged residues in the CY motifs for cyclin binding, and further indicate that the CY motifs function both independently and in tandem.

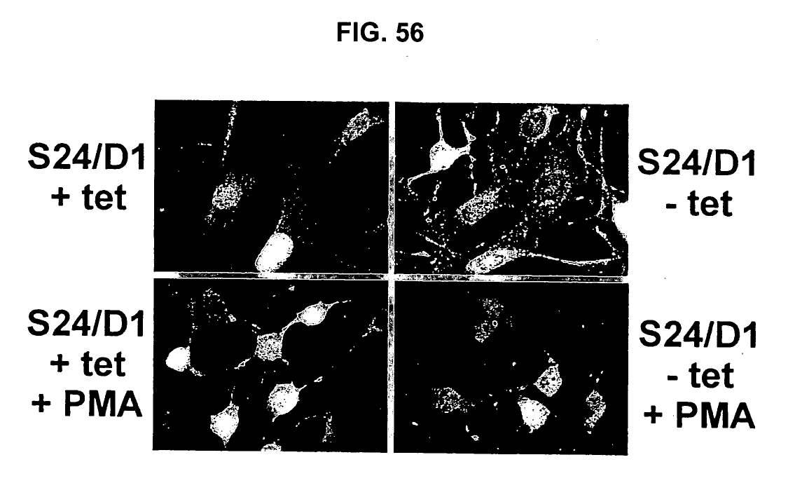

It is believed that SSeCKS′ scaffolding functions are down-modulated by kinases that are activated during G1→S progression. Nascent SSeCKS protein synthesized during early G1 or in confluent cultures is underphosphorylated, and following mitogenic stimulation, becomes rapidly serine phosphorylated (Nelson et al., 1997, Mol. Cell. Biochem. 175:233-241). Additionally, prephosphorylation of SSeCKS by PKC severely decreases its ability to bind phosphatidylserine and calmodulin (Lin et al., 1996, J. Biol. Chem. 271:28430-28438). Based on the finding that PKC-induced phosphorylation of GST-SSeCKS2 ablates in vitro binding activity to cyclins (above), it was determined whether the in vivo activation of PKC affects the putative binding of SSeCKS to cyclin D1. FIG. 56 and Table 9 show that the short-term addition of PMA to S24/D1 cells grown in the absence of tet caused a rapid translocation of cyclin D1 from cytoplasmic sites to the nucleus. Additionally, PMA induced SSeCKS translocation to perinuclear sites, as we and others showed previously (Chapline et al., 1998, J. Biol. Chem. 273:19482-19489; Gelman et al., 1998, Cell Motil. Cytoskeleton 41:1-17; and Lin et al., 1996, J. Biol. Chem. 271:28430-28438). These data indicate that some of SSeCKS′ in vivo scaffolding functions, possibly including binding to cyclins, are regulated by G1 phase phosphorylation.

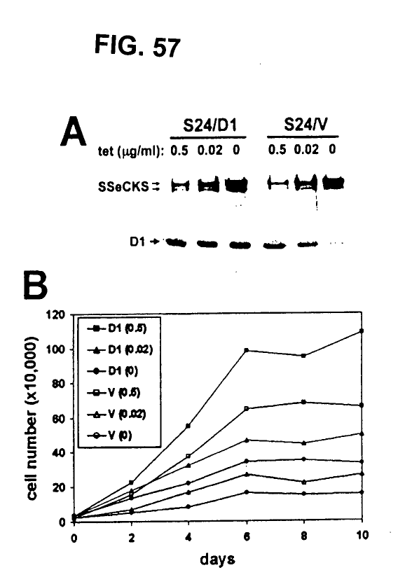

Decreasing the levels of ectopic SSeCKS rescues G1 arrest and the nuclear translocation of cyclin D1. In S24/D1 cells grown in the absence of tet, ectopic SSeCKS levels are more abundant than the levels of ectopic cyclin D1 (FIG. 57A). It was believed that decreasing the levels of SSeCKS should increase the ability of ectopic D1 to translocate to the nucleus and thus, rescue G1 arrest. To examine this possibility, the fact that the levels of ectopic SSeCKS could be modulated by varying the concentration of tet added was taken advantage of. FIG. 57A shows the effect of varying tet levels, such that at 0.02 μg/ml, the level of ectopic SSeCKS in both S24/V and S24/D1 cells was roughly equal to that in parental S6 cells. Most importantly, increasing the concentration of tet had no effect on the levels of ectopic D1 in S24/D1 cells but clearly caused an increase in the levels of endogenous D1 in S24/V cells due to a decrease in the ectopic levels of SSeCKS. FIG. 57B shows that at a tet concentration of 0.02 μg/ml, S24/D1 cells proliferated at a significantly greater rate than S24/V cells, which were still partially growth arrested in G1. Moreover, there is a direct correlation between the increasing levels of ectopic SSeCKS and increasing levels of cytoplasmic cyclin D1 in the S24/D1 cells (Table 10). It should be noted that S24/D1 cells could not be compared directly to parental NIH3T3/D1 cells because the latter lack the tTA-mediated effects on proliferation. These data support the notion that SSeCKS-induced growth arrest and cyclin D1 translocation is dependent on the expression level of SSeCKS.

TABLE 10
Increased levels of indeed ectopic SSeCKS correlates
with increased cytoplasmic cyclin D1 sequestration^{a}

| | | |
|---|---|---|
| Cell | TET concn | % of cells showing staining of: |

| type | (μg/ml) | Cystoplasm | Nucleus |
|---|---|---|---|
| S24/V | 0 | 3 ± 3 | 15 ± 4 |
| S24/V | 0.02 | 8 ± 4 | 34 ± 5 |
| S24/V | 0.5 | 7 ± 4 | 70 ± 4 |
| S24/D1 | 0 | 85 ± 6 | 32 ± 4 |
| S24/D1 | 0.02 | 29 ± 4 | 59 ± 4 |
| S24/D1 | 0.5 | 8 ± 6 | 96 ± 3 |

^{a}Cells grown on 22-mm² coverslips in medium containing various concentrations of TET for 2 days were fixed and stained for cyclin D1 using PAb, as described in Materials and Methods. Independent fields of cells were counted (total of 250 to 300 cells for each analysis). Note that the decrease in nuclear staining in the S24/V cells correlating with decreasing TET concentration relates to the SSeCKS-induced downregulation of cyclin D1 (FIG. 8A).

Penetratin-linked CY-encoding peptides compete for in vivo SSeCKS/cyclin D binding. Attempts to show co-immunoprecipitation from cellular lysates due to SSeCKS association with the cytoskeleton: mild lysis conditions (e.g.—0.5% NP-40) resulted in the non-specific coprecipitation of proteins with SSeCKS, whereas stronger detergent conditions (e.g.—RIPA) stripped off all interacting proteins. Moreover, coprecipitating overexpressed proteins does not reflect physiological conditions nor do they rule out the involvement of intermediary or “adaptor” proteins.

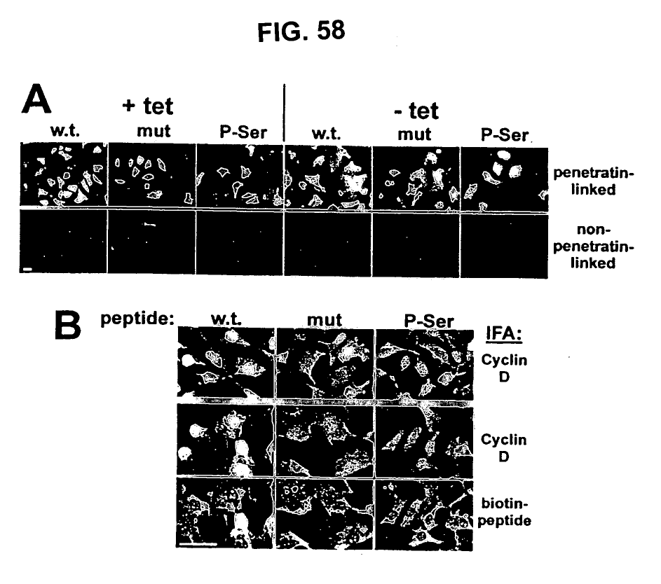

As an alternative approach to studying the in vivo interaction between SSeCKS and cyclin D1, we treated S24/D1 cells grown in the absence of tet with penetratin-linked peptides encoding either the wt SSeCKS CY domains, K→S mutants peptides or peptides with phospho-Ser^{507/515}. Penetratin corresponds to a homeodomain of the Drosophila Antennapedia transcription factor that facilitates the internalization of peptides, even phosphorylated versions (Peck and Isacke, 1998, J. Cell Sci. 111:1595-1601), and oligonucleotides into many cell types (Derossi et al., 1994, J. Biol. Chem. 269:10444-10450), possibly by the formation of inverted micelles (reviewed in Derossi et al., 1994, J. Biol. Chem. 269:10444-10450). FIG. 58A shows that biotin-labeled versions of the penetratin-linked CY peptides (wt, K→S mutation, or phospho-Ser507/515) entered S24/D1 cells with equal efficiency, indicating that the CY variations had no effect on penetratin-mediated transduction. In contrast, biotin-labeled CY peptides lacking the penetratin motif did not enter at all, indicating that transduction is penetratin-mediated. FIG. 9B and Table 5 show that incubation of S24/D1 cells for 4 h with the penetratin-[wt CY] peptide caused a roughly 2-fold increase in cyclin D translocation to nuclei whereas the penetratin-[K→S] or -[phospho-Ser^{507/515}] peptides or the peptides lacking the penetratin motif did not significantly decrease the cytoplasmic sequestration of D1 (Table 11).

TABLE 11
Percentage of cells showing nuclear staining of cyclin
D1 following treatment with penetratin-linked peptides^{a}

| Cell | | | % of cells showing |
|---|---|---|---|
| type | Peptide added | TET | nuclear staining |
| S24/D1 | — | + | 95 ± 3 |
| S24/D1 | — | − | 28 ± 4 |
| S24/D1 | Penetratin-wt CY | − | 82 ± 5 |
| S24/D1 | Penetratin-K→S | − | 30 ± 5 |
| S24/D1 | Penetratin-phospho- | − | 39 ± 4 |
| | Ser^{507/515} | | |
| S24/D1 | wt CY | − | 95 ± 4 |
| S24/D1 | K→S | − | 95 ± 5 |
| S24/D1 | Phospho-Ser^{507/515} | − | 91 ± 5 |

^{a}Cells grown on 22-mm² coverslips with (+) or without (−) TET for 3 days and then with or without peptide (100 μg/ml) for 4 h were fixed and stained for cyclin D1 using PAb, as described in Materials and Methods, Three independent fields of cells were counted (total of 250 to 300 cells for each analysis).

These data indicate that the wt CY peptide competes with the in vivo binding between SSeCKS and cyclin D, but that mutation of the basic KK residues or addition of phosphoserines in the CY domains ablates the competing activity.

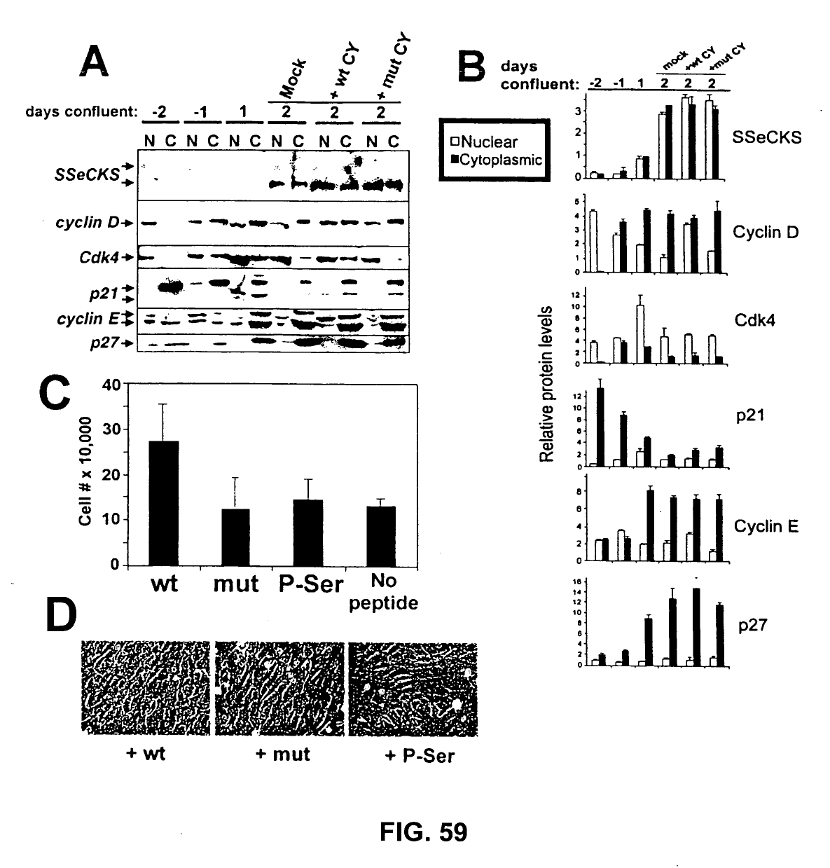

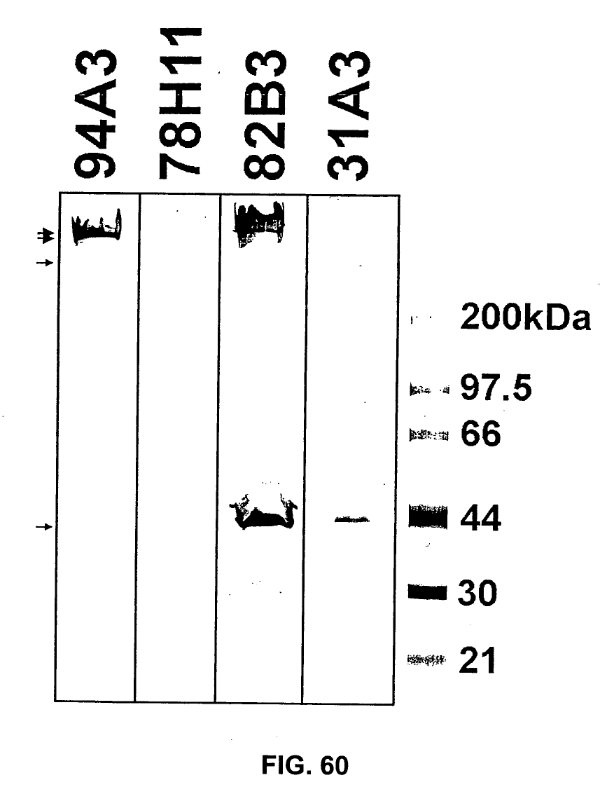

It was possible that the SSeCKS-induced sequestration of cyclins may be an artifact of SSeCKS overexpression. Thus, the level and compartmentalization of G1→S cyclins, CKIs and Cdk4 as cycling untransformed rat embryo fibroblasts transition to contact-inhibition. FIG. 59A confirms previous findings (Lin et al., 1995, Mol. Cell. Biol. 15:2754-2762; Nelson et al., 1997, Mol. Cell. Biochem. 175:233-241) that the level of SSeCKS protein is induced by confluency. Although some nuclear SSeCKS has been identified by confocal immunofluorescence microscopy (Gelman et al., 1998, Cell Motil. Cytoskeleton 41:1-17), much of the “nuclear” component here is attributed as perinuclear and/or cytoskeletal SSeCKS. Also, as described previously by others (Yanagisawa et al., 1999, J. Biochem. (Tokyo) 125:36-40), contact-inhibition results in increased p27 and decreased p21 levels relative to cycling cells. Much of the sustained levels of cyclin D in contact-inhibited populations is likely to be pre-existing protein because the level of cyclin D transcription in confluent cells drops precipitously. This agrees with the finding above that increased SSeCKS levels, whether ectopically induced by tet or endogenously induced by confluency, leads to inhibition of cyclin D transcription.

The data in FIG. 59A clearly indicate a cytoplasmic shift for p27 and cyclins D and E correlating with confluency. In contrast, the earliest stages of cell confluence (days −1 and 1) are marked by increases in Cdk4 level and an equilibrium between nuclear and cytoplasmic components with the majority of protein being nuclear. Lastly, cytoplasmic p21 levels, which are high in cycling cells, transiently shift to the nucleus at the onset of cell confluence.

To determine if SSeCKS plays a role in cytoplasmic sequestration during contactin-hibition, rat embryo fibroblasts kept confluent for two days were treated for 2.5 additional days with repeated doses of either the wt or mutant penetratin-CY peptides. FIGS. 59A and B show that the wt-CY peptide induced a 3-5 fold increase in nuclear cyclin D compared the mutant-CY peptide or mock treatment. This correlated with a 2-fold increase in cell density (FIG. 59C) in the absence of any increase in cell refractility (FIG. 59D). A marginal increase in nuclear cyclin E was also detected after wt-CY treatment. In contrast, the CY peptides had no effect on the localization of Cdk4, p21 or p27. These data clearly indicate that antagonism of a putative SSeCKS/cyclin D cytoplasmic complex leads to cyclin D nuclear translocation and an increase in saturation density.

The present invention is not to be limited in scope by the specific embodiments described herein Which are intended as single illustrations of individual aspects of the invention, and functionally equivalent methods and components are within the scope of the invention. Indeed, various modifications of the invention, in addition to those shown and described herein will become apparent to those skilled in the art from the foregoing description and accompanying drawings. Such modifications are intended to fall within the scope of the claims. Various publications are cited herein, the contents of which are hereby incorporated, by reference, in their entireties.

1. A monoclonal antibody which immunospecifically binds to an epitope of SSeCKS.

2. The monoclonal antibody of claim 1 which binds to human SSeCKS.

3. The monoclonal antibody of claim 1 produced by the hybridoma cell line designated 94A3.

4. The monoclonal antibody of claim 1 produced by the hybridoma cell line designated 78H11.

5. The monoclonal antibody of claim 1 produced by the hybridoma cell line designated 82B3.

6. The monoclonal antibody of claim 1 produced by the hybridoma cell line designated 31A3.

7. A method for identifying a compound capable of modulating cell proliferation comprising:
(i) contacting a cell that comprises a reporter gene under the transcriptional control of a SSeCKS gene responsive element with a test compound and measuring the level of reporter gene expression in the cell;
(ii) measuring the level of reporter gene expression in the absence of the test compound; and
(iii) comparing the levels of reporter gene expression measured in (i) and (ii);
wherein a difference in the levels of reporter gene expression measured in steps (i) and (ii) has a positive correlation with cell proliferation modulating activity of the test compound.

8. The method of claim 7 wherein an increase in reporter gene activity correlates with the ability of the test compound to inhibit cell proliferation.

9. A method for identifying a compound capable of modulating cell proliferation comprising:
(i) contacting a cell that comprises a reporter gene under the transcriptional control of a cyclin D gene responsive element with a test compound and measuring the level of reporter gene expression in the cell;
(ii) measuring the level of reporter gene expression in the absence of the test compound; and
(iii) comparing the levels of reporter gene expression measured in (i) and (ii);
wherein a difference in the levels of reporter gene expression measured in steps (i) and (ii) has a positive correlation with cell proliferation modulating activity of the test compound.

10. The method of claim 7, wherein the cell is a transformed cell.

11. The method of claim 10 wherein the cell is a ras or src transformed cell.

12. A method for identifying a compound capable of modulating hair growth comprising:
(i) contacting a cell that expresses cyclin D and SSeCKS, a in the presence of a stimulator of cell proliferation;
(ii) determining the level of nuclear translocation of the cyclin D into the nucleus of the cell;
(iii) determining the level of nuclear translocation in the absence of the test compound; and
(iv) comparing the level of nuclear translocation measured in (ii) and (iii);
wherein a difference in the level of nuclear translocation measured in steps (ii) and (iii) has a positive correlation with cell proliferation modulating activity of the test compound.

13. A method of inhibiting cell proliferation in a cell comprising introducing a nucleic acid molecule encoding a SSeCKS polypeptide that is capable of binding cyclin D and preventing translocation of cyclin D into the nucleus.

14. The method of claim 13 wherein the nucleic acid molecule further encodes a cytoskeletal anchoring peptide.

15. A method of inhibiting cell proliferation in a cell comprising introducing a nucleic acid molecule encoding a SSeCKS polypeptide that has an increased affinity for cyclin D.

16. A method for determining the metastatic potential of a cancer cell comprising:
(a) detecting the expression of SSeCKS in the cell; and
(b) comparing the level of SSeCKS expression in the cancer cell to the level of expression in a control sample;
wherein a decrease in the level of SSeCKS expression detected in the cancer cell as compared to the normal cell is an indicator of increased metastatic potential.

17. The method of claim 16 wherein the SSeCKS protein is detected using an immunoassay.

18. A method for determining the metastatic potential of a cancer cell comprising detecting the presence of a SSeCKS encoding nucleic acid in the cell; wherein a decrease or absence of SSeCKS encoding nucleic acid within the cell is an indicator of increased metastatic potential.

19. A method for modulating cell proliferation in a mammal comprising administering to the mammal a compound that prevents nuclear translocation of cyclin D.

20. A method for modulating hair growth in a mammal comprising administering to the mammal a compound that increases the expression of SSeCKS.
